SEQUENCE LISTING (11 sequences)

SEQ ID NO: 1 - DNA, 30690 bp, Streptomyces avermitilis
  CDS (1)..(11916)
  CDS (11971)..(30687)
gtgcagaggatggacggcggggaagaaccccgccctgcggcaggggaggtcctcggagtggccgacgaggcggacggcggcgtcgtcttcgtttttcccgggcagggcccgcaatggccgggcatgggaagggaacttctcgacgcttccgacgtcttccgggagagcgtccgcgcctgcgaagccgcgttcgcgccctacgtcgactggtcggtggagcaggtgttgcgggactcgccggacgctcccgggctggaccgggtggacgtcgtccagccgaccctgttcgccgtcatgatctccctggccgccctctggcgctcgcaaggggtcgagccgtgcgcggtgctgggacacagcctgggcgagatcgcggcagcccacgtctcgggaggcctgtccctggccgacgccgcacgcgtggtgacgctttggagccaggcacagaccacccttgccgggaccggcgcgctcgtctccgtcgccgccacgccggatgagctcctgccccgaatcgctccgtggaccgaggacaacccggcgcggctcgccgtcgcagccgtcaacggaccccggagcacagtcgtttccggtgcccgcgaggccgtcgcggacctggtggccgacctcaccgccgcgcaggtgcgcacgcgcatgatcccggtggacgttcccgcccactcccccctgatgtacgccatcgaggaacgggtcgtcagcggcctgctgcccatcaccccacgcccctcccgcatccccttccactcctcggtgaccggcggccgcctcgacacccgcgagctagacgcggcgtactggtaccgcaacatgtcgagcacggtccggttcgagcccgccgcccggctgcttctgcagcaggggcccaagacgttcgtcgagatgagcccgcacccggtgctgaccatgggcctccaggagctcgccccggacctgggcgacaccaccggcaccgccgacaccgtgatcatgggcacgctgcgccgcggccagggcaccctggaccacttcctgacgtctctcgcccaactacgggggcatggtgagacgtcggcgaccaccgtcctctcggcacgcctgaccgcgctgtcccccacgcagcagcagtcgctgctcctggacctggtgcgcgcccacaccatggcggtgctgaacgacgacggaaacgagcgcaccgcgtcggatgccggcccatcggcgagtttcgcccacctcggcttcgactccgtcatgggtgtcgaactgcgcaaccgcctcagcaaggccacgggcctgcggttgcccgtgacgctcatcttcgaccacaccacgccggccgcggtcgccgcgcgccttcggaccgcggcgctcggccacctcgacgaggacaccgcgcccgtaccggactcacccagcggccacggaggcacggcagcggcggacgacccgatcgccatcatcggcatggcatgccgtttcccgggcggagtccggtccccgaaggacctgtgggagctggccgcctcgggcggagacgccatcgggccgttccccaccgaccgcggatggcccacggaacagcgtcacgcccaggaccccacgcagcccggcacgttctatccgcagggaggcgggttccttcacgacgcggcgcacttcgacgccggcttcttcggaatcagtccacgtgaggcactggcgatggatccgcagcagcggctgctgctggagacgtcctgggaggcgttcgagcgggcgggaatcgatccgctgtcggtacgcgggtcccgtacgggcgtcttcgcgggcgccctctccttcgactacggcccgcgtatggacaccgcgtcgtcggagggcgccgcggacgtggagggccacatcctcaccggtaccacgggcagcgtcctgtcgggccgtatcgcctacagcttcgggctggaagggccggcgatcaccgtggacacggggtgctcggcatcgctcgtgacgctgcatctggcgtgccagtcgctgcggtcgggtgagtgcacgctcgcgctggccggcggcgtctcggtcatgtccaccctcggcatgttcatcgagttctcccggcagcgcgggctgtcggtggacggcaggtgcaaggcgtactcggctgcagccgacggcaccggctggggcgagggcgtcgggatgctgttggtggagcggttgtcggatgcggtgcggctggggcatcgggtgctggcggtggtacgcggcagtgcggtcaaccaggacggtgcgtcgaatgggctgacggcgccgaacggtccggctcaggagcgggtgatccggcaggcgttggcgaacgcggggttgtccgtggcggatgtggatgtggtggaggggcacgggacgggcacgacgctgggtgatccgatcgaggcacaggcgttgctcgccacgtacgggcagcgggccggtgacaggccgctgtggctggggtctctgaagtccaacatcgggcacaccatggctgccgcgggtgtgggtggggtcatcaagatggtgatggcgttgcgggagggggtgttgccgcggacgttgcatgtggataagccgtcgccgcaggtggactggtccgcgggggcggtgcggctgctgacggaggcggtgccgtggccgggggacgcggcagggcggttgcggcgggcgggagtgtcgtcgttcgggatcggcggcacgaatgcgcatgtgattttggaggaggcgccggcggcggggggctgtgttgccgggggtggggtgttggagggtgctccgggtcttgccatttcggtggctgagtcggtggccgctccagtggctgtgtctgcgccggtggctgagtcggtgccggtgccggtgccggtgccggttcctgtgccggtgtcggctaggtctgaggctgggttgcgggcgcaggcggaggcgttgcgtcagtacgtggcagtccggccggacgtttcgcttgccgatgtgggtgcgggtctggcctgtgggcgggctgtgctggagcatcgtgcggtcgtcctggccgcggaccgtgaggagctggtgcaagggttgggggcgctggcggcgggtgagccggatcggcgggtgaccacgggtcatgcgccgggtggtgaccggggcggtgtcgtcttcgtgtttcccggacagggtgggcagtgggccgggatgggtgtgcgtctgctcgcctcctctccggtgttcgcccggcggatgcaggcgtgcgaggaggctctggcgccgtgggtggactggtctgtggtggacatcctgcgccgggacgcgggggatgcggtgtgggagcgggccgatgtggtccagcctgtgctgttcagcgtcatggtgtctttggctgctctgtggcgttcctacggtatcgaacccgacgcggtccttggccattcccagggcgagatcgcggccgcgcatgtgtgtggggcgctgagcctgaaggacgcggcgaagactgttgcgctgcgcagccgggcgctggccgctgtgcggggccggggcggcatggcctcagtgccgctgcctgcccaggaggtggagcagctcattggtgagcggtgggcggggcggttgtgggtggcggcggtcaacggcccccgctccaccgccgtctcgggggatgccgaggcggtggacgaggtgctggcgtactgtgccggcaccggggtgcgggcccggcggatcccggtcgactatgcctcgcactgcccccatgtgcagcccctgcgggaggagttgctggagctgctgggggacatcagcccgcagccgtccggcgtgccgttcttctccacggtggagggcacctggctggacaccacaaccctggacgccgcctactggtaccgcaacctgcaccagccggtccgtttcagcgatgccgtccaggccctggcggatgacggacaccgcgtcttcgtcgaagtcagcccccaccccaccctcgtccccgccatcgaagacaccaccgaagacaccgccgaagacgtcaccgcgatcggcagcctccgccgcggcgacaacgacacccgccgcttcctcaccgccctcgcccacacccataccaccggcatcggcacacccaccacctggcaccaccactacacccaccaccacacccacccccacccccacacgcacctcgacctgcccacctaccccttccaacaccagcactactggctcgagagctcacagccgggtgccggatccggttcgggtgccggtgccggttcgggtgccggttccgggcgggcagggactgcgggcgggacggcagaggtggagtcgcggttctgggacgcggtggcccgccaggacctggaaacggtcgcgaccacactcgccgtgcccccctccgccggcctggacacggtggtgcccgcactctccgcctggcaccgccaccaacacgaccaagcccgcatcaacacctggacctaccaggaaacctggaaacccctcaccctccccaccacccaccaaccccaccaaacctggctcatcgccatccccgaaacccagacccaccacccccacatcaccaacatcctcaccaacctccaccaccacggcatcacccccatccccctcaccctcaaccacacccacaccaacccccaacacctccaccacaccctccaccacacccgacaacaagcccaaaaccacaccaccggagccatcaccggcctgctctccctcctcgccctcgacgaaacaccccacccccaccacccccacacacccaccggcaccctcctcaacctcaccctcacccaaacccacacccaaacccacccaccaacccccctctggtacgccaccaccaacgccaccaccacccaccccaacgaccccctcacacaccccacccaagcccaaacctggggactcgcccgcaccaccctcctcgaacaccccacccacaccgccggaatcatcgacctccccaccacccccaccccccacaccctccagcacctcacccaaaccctcacccaaccccaccaccaaacccaactcgccatccgcaccaccggcacccacacccgccgcctcacccccaccaccctcacccccacacaccaaccacccacccccaccccccacggaaccaccctcatcaccggcggaaccggcgccctcgccacccacctcacccaccacctcaccacccaccaacccacccaacacctcctcctcaccagccgaaccggcccccacaccccccacgcacaacacctcaccacccaactccaacaaaaaggcatccacctcaccatcaccacctgcgacaccagcaacccagaccaactccaacaactcctcaacaccatccccccacaacaccccctcaccaccgtcatccacaccgcaggcatcctcgacgacgccaccctcaccaacctcacccccacccaactcaacaacgtcctccgcgccaaagcccacagcgcccacctcctccaccaactcacccaacacacccccctcaccgccttcgtcctctactcctccgccgccgccaccttcggcgcacccggccaagccaactacgccgcagccaacgcctacctcgacgccctcgcccaccaccgccacacccaccacctccccgccaccagcatcgcctggggcacctggcaaggaaacggactcgctgattcggacaaggcccgcgcatatctcgaccgccgcgggtttcgacccatgtcacccgagttggccacggcagcggtcacgcaggcgatcgcggacaccgaacggccgtatgtcgtcatcgccgacatcgactggagcaagatcgaacacacctctcagaccagcgacctggtgagcgcggcccgggaaagggagccagctgtccagcgccccactccaccggcggagttgcacaaaacgctggcccatcagacgtcggccgaccaacgggccgcattgctcgagctcgtacgagaccatgtggcggcagtgctccggcacgcggacccgaaagccatcgcgcccgaccagtcgttccgtgcactcggcttcgattcactcacggccgtcgagttccgaaacctgctgatcaaggcaacaggactccgccttcctgtctcgctggtcttcgaccacccgacccctgccaaactcgccgtacacctgcagaaccaactgcggggcacagcagcggagtcggctccttcagcggcagccgttaccgccgaggcttctgtcaccgagccgatcgccatcgttggcatggcctgtcgtttccccggcggagtgacctcggcggacgacttctgggatctgatctcctccgagcaggacgcgatcggcggattccccaccgaccgcggctgggacctggacacgctctacgaccccgaccccgaccaccccggcacctgctacacccgaaacggcggattcctctacgacgcaggccacttcgacgccgaattcttcggcatcagcccccgcgaagccctcgccatggacccccagcaacgactcctcctcgaaaccgcctgggaaaccatcgaacacgccggcatcaacccccacaccctccacggcacccccaccggagtcttcaccggcaccaacggacaggactacgcacttcgcgtgcacaacgcgggccagtcaaccgatggtttcgcactgaccggaaccgccggcagcgtcatctccggtcgtatctcgtacacgtttggttttgagggtcctgcggtgtcggtggacacggcttgttcctcgtcgttggtggctttgcatctggcctgtcaggcgttgcgtgcgggtgagtgctcgatggcgcttgccgggggtgtgacggtgatgtcgtctccgggtgccttcgtggagttttcgcggcagcggggtctggccgcggacgggcattgcaaggcgttctcggcggcggcggacgggaccggctggggtgagggtgtggggatgctgctggtggagcggctctccgacgcccatcgcaacggtcaccgtgtcctggccgtggtgcgtggcagtgcggtcaaccaggacggtgcgagcaacggtctgaccgcgcccaacgggccgtcccagcagcgtgtcatccgccaggccctcgccaacgccggcttgtcggccggtgatgtcgacgcggtggaggcccacggcaccggcaccactttgggcgacccgatcgaggcccaggccctcctcgcgacctacggacaggaccgtgccggcgaggggccgctgtggctgggctcggtcaagtccaatgtcggtcacacacaggctgccgcgggcgtcgccggggtgatcaagatggtgatggcgctgcggcatggtctgctgccgcggacgttgcatgtggatgagccgtcgccgcatgtggactggtccgcgggtgcggtgcagctgctgacggagacggtgccctggcccggcggggaggggcggctacggcgggcaggagtgtcatcattcggcgtcagcggcaccaacgcccacgtcatcctcgaagaagcacccgccgacgacgttccggggggaccacccgccggcgagggtgacgcgggcagcgacgatgaggctgctgccggcagtcctggggtgtggccgtggctggtgtcggccaagtcgcagccggccctgcgcgcccaggcccaggccctgcacgcccacctcaccgaccaccccggcctcgacctcgcggatgtcggatacaccctcgcccacgcccgcgccgtgttcgaccaccgcgccaccctcatcgccgcggaccgcgacacgttcctgcaagcactccaggcactcgccgcaggcgagccccaccccgccgtcatccacagcagcgccccgggcgggaccgggaccggggaggccgcaggaaagaccgcattcatctgctccggacagggcacccaacgccccggcatggcccacggcctctaccacacccaccccgtcttcgccgccgcactcaacgacatctgcacccacctcgacccccacctcgaccaccccctcctccccctcctcacccaaaacgacaacgacaacgaggacgcggccgcactgctccagcagacccgctacgcccagcccgccctcttcgccttccaggtcgccctccaccgcctcctcaccgacggctaccacatcaccccccactactacgccggacactccctcggcgaaatcaccgccgcccacctcgccggcatcctcaccctcaccgacgccaccaccctcatcacccaacgcgccaccctcatgcaaaccatgccccccggcaccatgaccaccctccacaccaccccccaccacatcacccaccacctcaccgcccacgaaaacgacctcgccatcgccgccatcaacacccccacctccctcgtcatcagcggcaccccccacaccgtccaacacatcaccaccctctgccaacaacaaggcatcaaaaccaaaaccctccccaccaaccacgccttccactccccccacaccaaccccatcctcaaccaactccaccagcacacccaaaccctcacctaccacccaccccacacccccctcatcaccgccaacaccccacccgaccaactcctcaccccccactactggacccaacaagcccgcaacaccgtcgactacgccaccaccacccaaaccctccaccaacacggcgtcaccacctacatcgaactcggacccgacaacaccctcaccaccctcacccaccacaacctccccaacccccccaccaccaccctcaccctcacccacccccaccaccacccccaaacccacctcctcaccaacctcgccaaaaccaccaccacctggcacccccaccactacacccaccacgacaaccaaccccacacccacacccacctcgacctccccacctaccccttccaacaccaccactactggctcgaaagcacacagcccggtgccggcaacgtgtcagcagccggactcgaccccaccgaacaccccctactcggcgccacattggaactggcgactgacggtggagcgcttcttgcagggcgcttgtctttgaggtcgcatccgtggctggctgaccatgccgtcggcggcacggtgctgctgtcgggcgccaccttcctcgaactcgcccttcatgcgggcacatacgtgggctgcgaccgagtggatgagctgacgctgcatgcgccgctggtggttcctgtggatgggggtgtgagtgtgcaggttggggttgcggctgcggatggggaggggcggcgtttggtgagtgtgtatgcgcggggtgggagtgcttgtggtgggggtggtgcgtcgggtggggtgtggacgtgtcatgcctcgggggtgctggttgaggctgctgctggtggtgtggtggtggatggtctggcgggggtgtggccgccgcggggtgcggtggcggtggatgtcgatggtgtccgtgaccgtttggctggggctggttgtgttttggggccggtgttttcggggctgcgtgcggtgtggcgtgatgggggggatttgctggctgaggtgtgtctgccggaggaggcgtggggtgatgcggctggttttgggctgcatccggcgttgctggatggtgtggtccagccgttgtcggtgttgcttccgggtgggacggggtttggggagggggcggggttcggggagggtgttcgggtgccggctgtgtggggtggtgtgtcgcttcaccgggcgggtgtgaccggtgtgcgggtgcgtgtgtcggctgtcgggcggggcggcgggcgtgaggcggtgtcggtcgtggtcggggatgaggcgggtgtgccggtggcgtcggtcgatcgtcttgagttgcggcctgtggatatgggtcagttgcgtgctgtctcggtttcggcggggcggcggggttcgctgtatgcggtgcagtgggctgaggtgggtcctgtgccggtgtgtgggcaggcgtgggcgtggcacgaggacgtgggtgagagcggtggtgggcctgtgccgggggtggtggtgttgcggtgcccggatgccggtgccggtggcggtggcggtggcggtggtggcggtggtgtgggtgaggttgttggtggggtgttgggtgtggtgcaggggtggctggggctggagcggtttgcgggttcgcggctggtggtggtgacccggggtgcggtggtggccggcccggaggacggcccggtggatgtggtgggtgcgtcggtgtgggggctggtgcgttcggcgcaggctgagcatccggaccggtttgtcctcctcgacctcgacaccgacaccggcaccgacctcgacaccggtgctggtgctggttggggcgtggatggtgggcgtgtggcggcggtggtggcgtgtggtgagccgcagttggcggtgcgtggggagcggttgctggccgcacgcctgaaacgacttgagtcatccggtgatgttccagcccagcggtccggtgacacacgagcccggcggtccgacgtgcctgcccagcgctccggtggcgtgcctgctcggcggtcggttgatgtatcgggtcgggaggtgttgccgtggttgtcgggtgggtcggtgttggtgacgggtgggacgggtgtgctgggtgcggcggtggcgcggcatctggctggtgtgtgtggggtgcgggatctgctgttggtgagccggcgtggtccggatgctccgggtgcggagggtctgcgggcggagctggccgcgttgggggcggaggtgcggattgttgcgtgtgatgtgggggagcggcgggaggtggtccggctgctggagggtgttcctgccgggtgtccgctgacgggtgtcgtgcatgcggctggtgtgctggacgatgcgacgatcgcctctctcacgcccgagcggctgggcacggtgttcgcggccaaggtggatgccgctcttttgctggatgagctgacgcggggtatggagctgtcggcgttcgtgctgttctcctcggccgcggggatcctggggtcggccgggcagggcaactacgccgcggccaatgccgctctggacgcgctggcgtaccggcggcgggcggcgggtctgccgggggtgtcgctggcgtgggggctgtgggaagaggccagcgggatgaccgggcacctggccggcaccgaccaccggcgcatcatccgttccggtctgcatcccatgtcgaccccggacgcactggccctcttcgatgcggccctggctctggaccggccggtcctgctgcccgccgacctgcgtcccgccccgcccctgccgcccctgctgcaggacctcctgcccgccacccgccgccgcaccacccgcaccaccactaccggtggtgcggacaacggcgcccagctgcacgcccggctggccggccagacacacgaacaacagcacaccaccctcctcgccctggtccgctcccacatcgccaccgtcctgggccacaccacccccgacaccatcccccccgaccgcgcgttccgcgacctcggcttcgactccctcaccgccgtcgaactacgcaaccggctctcccgcaccaccggactccgcctccccaccaccctcgccttcgaccaccccaaccccaccaccctcacccaccacctccacacacaactccagccacaaccggacaacgctgtcgcccccgtgttggcggagctcgacaaactcgaatccgccctctccgccctcgacaaaaccgacagcgccagcgaaagagtcaccctgcggctgaagtcactcatgttgaggtggaacgcaccccagcatccgacagccgaaagcgctgatgacgacgagaagttcacatcggcaacagaggctgagattttcaaattcattgacaacgacctcggcctgtcctgaaccggacgcctgccactccgcccgtatccgctgggccctgctaggacgtgaatgcaattggcgaatgaagcgaagctcctggaatacctcaagcgcgtcactgcggacctggaccgcactcgccgtcgcctgtacgaggtggtcgagcgtgagcaggagccgatcgcgattgtggggatggcgtgtcgttacccaggcggggcgacgtcacccacgcgactgtggcatctcgtcaagtcccagacggacgctatcggggagttcccgaccgaccgtggatggaacctggagcagctctacgacccggaccccgaccgctcaggaaccagttacacgcgcagcggagggtttctctatgacgcgggcgacttcgacgccgcgttcttcgagttgtcaccgcgtgaggcgctggcaatggacccgcagcagcgcctgctgctcgaaaccacttgggaaacgttcgaacagggcggaatcgacccgaggtccatgcgcggaagccggaccggggttttcgtggggatcaatccggaggactacaccaccggatacacacatcagccctcaaacgcagtcgagggctacctgctcactggcagcgcggcaagcattgcgtcaggccgtatctcctacaacttcgggctcgaaggccctgcgatcactatcgacaccgcgtgttcctcctcgctcgtcgccctgcatctggcctgccaagcgctccggtccggtgaatgcaccatggcgctcgcaggcggcgcctccgtcatggccactcccttcgtcttcaccgagttctctcgccagcggggcctggccgcagacggccggtgcaaggcgttttcggcggcggcggacgggaccggctggtccgagggtgtggggatgctgctggtggagcggctctccgacgcccgccgcaacggtcaccgtgtcctggccgtcgtccgcggcagcgccgtcaaccaggacggcgcaagcaacggcctgaccgcacccaacggtcgttcacaagtcaaggtcatccgccaggctttggccaacgcacacctctcccctgccgatgtcgatgcggtggaggcccacggcacggggaccaccctgggcgacccgatcgaggctcaagccctcgtcgaagcctacggtcaggaccgccccaacggccgccccctctggctcggaaccctcaagtccaacatcgggcactccatggccgctgcgggtgtgggcggggtcatcaagatggtgatggcgctgcggaatggtctgctgccgcggacgttgcatgtggatgagccgtcgccgcatgtggactggtccgcgggtgcggtgcagctgctgacggagacggtgccctggcccggcggggaggggcggctacggcgggcaggagtgtcatcattcggcgtcagcggcaccaacgcccacgtcatcctcgaggaagcacccgcccacaacatcccgtcagacacacccgccgacgacgtcccgggagaatcagccgccgacgaggatgccggtagtggcgatgaggctgctgccggcagtccaggggtgtggccgtggctggtgtcggccaagtcgcagccggccctgcgcgcccaggcccaggccctgcacgcccacctcaccgaccaccccggcctcgacctcgccgacgtcgggtacaccctcgcccacgcccgcgccgtgttcgaccaccgcgccaccctcatcgccgccgaccgcgacaccttcctgcaagcactccaggcactcgccgcaggcgaaccccaccccgccgtcatccacagcagcgccccaggcgggaccgggaccggggaggccgcaggaaagaccgcattcatctgctccggacagggcacccaacgccccggcatggcccacggcctctaccacacccaccccgtcttcgccgccgcactcaacgacatctgcacccacctcgacccccacctcgaccaccccctcctccccctcctcacccaggaccccaacacccaggacaccaccaccctcgaagaagcggccgcactgctccagcagacccgctacgcccagcccgccctcttcgccttccaggtcgccctccaccgcctcctcaccgacggctaccacatcaccccccactactacgccggacactccctcggcgaaatcaccgccgcccacctcgccggcatcctcaccctcaccgacgccaccaccctcatcacccaacgcgccaccctcatgcaaaccatgccccccggcaccatgaccaccctccacaccaccccccaccacatcacccaccacctcaccgcccacgaaaacgacctcgccatcgccgccatcaacacccccacctccctcgtcatcagcggcaccccccacaccgtccaacacatcaccaccctctgccaacaacaaggcatcaaaaccaaaaccctccccaccaaccacgccttccactccccccacaccaaccccatcctcaaccaactccaccagcacacccaaaccctcacctaccacccaccccacacccccctcatcaccgccaacaccccacccgaccaactcctcaccccccactactggacccaacaagcccgcaacaccgtcgactacgccaccaccacccaaaccctccaccaacacggcgtcaccacctacatcgaactcggacccgacaacaccctcaccaccctcacccacgacaacctccccaacacccccaccaccaccctcaccctcacccacccccaccaccacccccaaacccacctcctcaccaacctcgccaaaaccaccaccacctggcacccccaccactacacccaccaccacaaccaaccccacacccacacccacctcgacctccccacctaccccttccaacaccaccactactggctccaaccacccggcaagccgagcgacccgtcaccgagcgaaggccgtgagcaagccacgaccccatcaaccccgctgcgtgatgtcctcgtgggcaagtctccgcaggagcgagacgaagagctgttgcgcctggtgcgcacccatgcggccgctgtgctgggccatgccactcccgaagtgatcgttccgaacaaggccttcaaagagctgggttttgattctctcgccgcaattcagcttcgtaatcgactgcttgctgacgttgacctgccgcttccggccacgctgatcttcgattaccccactccgatggcgctttgccagttcctccgggcggcgatcgtcggagcggacacaggcacgaccactcgtctgccgctaactgcggtccccgccgacgagccgatcgccatcgtcggcatggcctgtcggtaccccggtgatgtacggacggtcgatgatctctggcaggtggtcagtggtggccatgacgcgatcggcggattcccgacgaaccgtgggtgggacctcgacacgctgtacaacccggacccggaccaccacggaaccagctacacccggagcggcggattcctttacgacgcaggcaatttcgatcccgacttcttcggtatcagtccgcgtgaggcactggcgatggacccgcagcagcggctgctgctggaaacagcgtgggagagcatcgaacacgcctgcatcaaccccgacagcctccgtggcacaccaaccggcgtcttcgccgggctgacctaccacgactacgccgcgcgctttcccacagctccggcagggttcgaggggtatctcgggcacggaagcgcaggcagtatcgcctcgggtcgtgtcgcctacgctctcggcctggaaggtccggccctcacagtcgacactgcctgctcttcgtccctggtcgctctgcacctggcctgtcaggcgctgcggtccggcgagtgttccatggccctcgcgggtggcgtcacggtgatgtcaaccccggccgggttcgtggagttttcgcggcagcggggcctggccgtggacgggcggtgcaaggcgttctcggcagcggctgacggcaccggctggggtgagggtgtcggaatgctgctggtggagcggctgtcggacgcgcggcggctcggtcaccgaatcctcgcggtggtgcgtggcagtgcggtcaatcaggacggtgcgagcaacgggctgacggcgcccaacgggccgtcccaggagcgtgtcatccgcctggccctggccaacgcggacctgacccccgccgacgtcgatgcggtggaggcccacggcaccggcaccactttgggcgacccgatcgaggcccaggccctcctcgccacctacggacaggaccgccccggcaacgaaccgctgtggctgggctcgatgaagtcgaacatcggccacgcgcaggctgccgcaggtgtgggcggggtcatcaagatggtgatggcgctgcggaatggtctgctgccgcggacgttgcatgtggatgagccgtcgccgcatgtggactggtccgcgggggcggtgcagctgctgacggagacggtgccctggcccggcggggaggggcggctgcggcgggcaggagtgtcatcgttcggcgtcagcggcaccaacgcccacgtcatcctcgaagaagcacccgcccacaacatcccgtcagacacacccgccgacgacgccccgggagaagcagccgccgacgatgttccgggggaagcggccggcgacgacgccggtaccggcggggaagcgactggtcctgctgccggcagtccaggggtgtggccgtggctggtgtcggccaagtcgcagccggccctgcgcgcccaggcccaggccctgcacgcccacctcaccgaccaccccggcctcgacctcgccgacgtcgggtacaccctcgcccacgcccgcgccgtgttcgaccaccgcgccaccctcatcgccgccgaccgcgacaccttcctgcaagcactccaggcactcgccgcaggcgaaccccaccccgccgtcatccacagcagcgccccaggcgggaccgggaccggggaggccgcaggaaagaccgcattcatctgctccggacagggcacccaacgccccggcatggcccacggcctctaccacacccaccccgtcttcgccgccgcactcaacgacatctgcacccacctcgacccccacctcgaccaccccctcctccccctcctcacccaggaccccaacacccaggacaccaccaccctcgaagaagcggccgcactgctccagcagaccccgtacgcccagcccgccctcttcgccttccaggtcgccctccaccgcctcctcaccgacggctaccacatcaccccccactactacgccggacactccctcggcgaaatcaccgccgcccacctcgccggcatcctcaccctcaccgacgccaccaccctcatcacccaacgcgccaccctcatgcaaaccatgccccccggcaccatgaccaccctccacaccaccccccaccacatcacccaccacctcaccgcccacgaaaacgacctcgccatcgccgccatcaacacccccacctccctcgtcatcagcggcaccccccacaccgtccaacacatcaccaccctctgccaacaacaaggcatcaaaaccaaaaccctccccaccaaaaacgccttccactccccccacaccaaccccatcctcaaccaactccaccagcacacccaaaccctcacctaccacccaccccacacccccctcatcaccgccaacaccccacccgaccaactcctcaccccccactactggacccaacaagcccgcaacaccgtcgactacgccaccaccacccaaaccctccaccaacacggcgtcaccacctacatcgaactcggacccgacaacaccctcaccaccctcacccaccacaacctccccaacacccccaccaccaccctcaccctcacccacccccaccaccacccccaaacccacctcctcaccaacctcgccaaaaccaccaccacctggcacccccaccactacacccaccaccacaaccaaccccacacccacacccacctcgacctccccacctaccccttccaacaccagcactactggctcgaaagcacacagccgggtgccggatccggttcgggttccggttccgggcgggcagggactgcgggcgggacggcagaggtggagtcgcggttctgggacgcggtggcccgccaggacctggaaacggtcgcgaccacgctcgccgtgcccccctccgccggcctggacacggtggtgcccgcactctccgcctggcaccgccaccaacacgaccaagcccgcatcaacacctggacctaccaggaaacctggaaacccctcaccctccccaccacccaccaaccccaccaaacctggctcatcgccatccccgaaacccagacccaccacccccacatcaccaacatcctcaccaacctccaccaccacggcatcacccccatccccctcaccctcaaccacacccacaccaacccccaacacctccaccacacccgacaacaagcccaaaaccacaccaccggacccatcaccggcctgctctccctcctcgccctcgacgaaacaccccacccccaccacccccacacacccaccggcaccctcctcaacctcaccctcacccaaacccacacccaaacccacccaccaacccccctctggtacgccaccaccaacgccaccaccacccaccccaacgaccccctcacacaccccacccaagcccaaacctggggactcgcccgcaccaccctcctcgaacaccccacccacaccgccggaatcatcgacctccccaccacccccaccccccacaccctccaccacctcacccaaaccctcacccaaccccaccaccaaacccaactcgccatccgcaccaccggcacccacacccgccgcctcacccccaccaccctcacccccacacaccaaccacccacccccaccccccacggaaccaccctcatcaccggcggaaccggcgccctcgccacccacctcacccaccacctcaccacccaccaacccacccaacacctcctcctcaccagccgaaccggcccccacaccccccacgcacaacacctcaccacccaactccaacaaaaaggcatccacctcaccatcaccacctgcgacaccagcaacccagaccaactccaacaactcctcaacaccatccccccacaacaccccctcaccaccgtcatccacaccgcaggcatcctcgacgacgccaccctcaccaacctcacccccacccaactcaacaacgtcctccgcgccaaagcccacagcgcccacctcctccaccaactcacccaacacacccccctcaacgccttcgtcctctactcctccgccgccgccaccttcggcgcacccggccaagccaactacgccgcagccaacgcctacctcgacgccctcgcccaccaccgccacacccaccacctccccgccaccagcatcgcctggggcacctggcaaggaaacggactggcgactggtcaagtcagcgaacatctccgccgccgcgggatgttcgccatgccgcccgagttggcggtcacagctgttgacggcgcgatcgcgagcgggcgcccgagtctcctcgtcgccgatatcgactggaagaaattgggaccggttctctccagcaagtcgtcggtcttgctcgaggaccttccccaggcacagggaactgaggaggcgcgcagtaccgttgagcagacggagagcacaaacctccggcaactcctcatgggtcggtcacgttccgagcaggaagaagagctgctcagcctcgtccgcatccactccgcggcagtgctcgggcgcgacgactccgaggccatcccgcccggtcggctgttcagggatctagggttcgactcgcttgcggcggtggagcttcgcaaccacctcgcagcacagacggagctggctctgccgacgactctcgtcttcgattaccccagccccaccaagctcgcccaatttctgctctccgagatcgcggagttccagcccgacaactcaactccgcttccgcgaccccgggcagagctcgatgagccgatcgccatcgttggcatggcctgtcgcttccccggcggagtgacctcggcggacgacttctgggatctgatctcctccgagcaggacgcgatcggcggattccccaccgaccgcggctgggacctggacacgctctacgaccccgaccccgaccaccccggcacctgctacacccgaaacggcggattcctctacgacgcaggccacttcgacgccgaattcttcggcatcagcccccgcgaagccctcgccatggacccccagcaacgactcctcctcgaaaccgcctgggaaaccatcgaacacgccggcatcaacccccacaccctccacggcacccccaccggagtcttcaccggcaccaacggacaggaccacgcggcacacatccgtcaggccccgagcggtaccgagggattcgtcctgaccggggcagccaccagcatcgcctccggccgaatctcctacatcctcgggttggaagggcctgcggtcaccctcgacacagcgtgttcctcctcgctcgtcgccctgcacctcgcctgccagtccctcaggtccggtgaatgcaccatggccttggccggcggggccacggtcatgaccaccccgatcaccttcaccgaattcgcccgccaacgcggactcgcccccgacgggcgttgcaaggcgttctcggcggcggctgacggtaccggctggggtgagggtgtggggatgctgctggtggagcggctctccgacgcccgccgcaacggtcaccgtgtcctggccgtggtgcgtggcagtgcggtcaaccaggacggtgcgagcaacggtctgaccgcgcccaacgggccctcccagcagcgcgtcatccgccaggccctcgccaacgcggacctgacccccgccgacgtcgatgcggtggaggcccacggcaccggcaccactttgggcgacccgatcgaggcccaggccatcctcgcgacctacggacaggaccgtcccggcaacgggccgttgtggctgggctccgtcaagtccaacgtcggacacacacaggccgcggcgggcgtggccggagtgatcaagatggtgatggccctccgccaccggacactcccaccgactctccacgcggatgagccgtcgccgcatgtggactggtccgcgggtgcggtgcagctgctgacggagacggtgccctggcccggcggggaggggcggccgcggcgggcaggagtgtcatcattcggcgtcagcggcaccaacgcccacgtcatcctcgaagaagcacccgccgacgacgttccggggggaccacccgccgacgaggatgccggtagtggcgaggaggctgctgccggcagtcctggggtgtggccgtggctggtgtcggccaagtcgcagccggccctgcgcgcccaggcccaggccctgcacgcccacctcaccgaccaccccggcctcgacctcgccgacgtcggatacaccctcgcccacgcccgcgccgtgttcgaccaccgcgccaccctcatcgccgccgaccgcgacaccttcctgcaagcactccaggcactcgccgcaggcgaaccccaccccgccgtcatccacagcagcgccccaggcgggaccgggaccggggaggccgcaggaaagaccgcattcatctgctccggacagggcacccaacgccccggcatggcccacggcctctaccacacccaccccgtcttcgccgccgcactcaacgacatctgcacccacctcgacccccacctcgaccaccccctcctccccctcctcacccaaaacgacaacgacaacgacaacgaggacgcggccgcactgctccagcagaccccgtacgcccagcccgccctcttcgccttccaggtcgccctccaccgcctcctcaccgacggctaccacatcaccccccactactacgccggacactccctcggcgaaatcaccgccgcccacctcgccggcatcctcaccctcaccgacgccaccaccctcatcacccaacgcgccaccctcatgcaaaccatgccccccggcaccatgaccaccctccacaccaccccacaccacatcacccaccacctcaccgcccacgaaaacgacctcgccatcgccgccatcaacacccccacctccctcgtcatcagcggcaccccccacaccgtccaacacatcaccaccctctgccaacaacaaggcatcaaaaccaaaaccctccccaccaaccacgccttccactccccccacaccaaccccatcctcaaccaactccaccagcacacccaaaccctcacctaccacccaccccacacccccctcatcaccgccaacaccccacccgaccaactcctcaccccccactactggacccaacaagcccgcaacaccgtcgactacgccaccaccacccaaaccctccaccaacacggcgtcaccacctacatcgaactcggacccgacaacaccctcaccaccctcacccaccacaacctccccaacacccccaccaccaccctcaccctcacccacccccaccaccacccccaaacccacctcctcaccaacctcgccaaaaccaccaccacctggcacccccaccactacacccaccaccacaaccaaccccacacccacacccacctcgacctccccacctaccccttccaacaccaccactactggctcgaactacccagcgcccaaaccagccccggtcaaaggcgttctcgccgctcggctccagacaccgccgagtcggagttctgggacgcggtgaacgaggaagacctccagagcctcgccgaaaccctcgacatcgacgcctctgctctggacacggtggtgcccgcactctccgcctggcaccgccaccaacacgaccaagcccgcatcaacacctggacctaccaggaaacctggaaacccctcaccctccccaccacccaccaaccccaccaaacctggctcatcgccatccccgaaacccagacccaccacccccacatcaccaacatcctcaccaacctccaccaccacggcatcacccccatccccctcactgtcaaccacacccacaccaacccccaacacctccaccacaccctccaccacacccgacaacaagcccaaaaccacaccaccggacccatcaccggcctgctctccctcctcgccctcgacgaaacaccccacccccaccacccccacacacccaccggcaccctcctcaacctcaccctcccccaaacccacacccaaacccacccaccaacccccctctggtacgccaccaccaacgccaccaccacccaccccaacgaccccctcacacaccccacccaagcccaaacctggggactcgcccgcaccaccctcctcgaacaccccacccacaccgccggaatcatcgacctccccaccacccccaccccccacaccctccaccacctcacccaaaccctcacccaaccccaccaccaaacccaactcgccatccgcaccaccggcacccacacccgccgcctcacccccaccaccctcacccccacacaccaaccacccacccccaccccccacggaaccaccctcatcaccggcggaaccggcgccctcgccacccacctcacccaccacctcaccacccaccaacccacccaacacctcctcctcaccagccgaaccggcccccacaccccccacgcacaacacctcaccacccaactccaacaaaaaggcatccacctcaccatcaccacctgcgacaccagcaacccagaccaactccaacaactcctcaacaccatccccccacaacaccccctcaccaccgtcatccacaccgcaggcgtcaatctcttcgcccccgtgtcggaaaccgatgccgaatccttctcttccgttacggcagcgaaggcaacgggcgcggcgattctgcatgagttgctgctggaccatgaaacgcttgaacacttcattctcttctcgtcgggcgccggcgcttggggcagcgggaatcagtgcgcatactcggcggccaacgcatacctggacgcgctcgcgacgcatcgtcagacacatggacttcccggggcatcgatcgcctggggcccctgggccggaaagggcatgtcggccggtgatgcggctcatggttacctggaaaagcgcggcattctgccgatggagccacgcatggcgctcgcggcattccatcgtgcgcgggcgcagcggccgaattccaacctgatcatcgcggacatcgactgggagcgcttcgtccccgccttcaccgctcgacgccacagcccgctcatcgaggacattccggaggttcggcaagcggctcaggagctggaagcagctgcgtcgacggcaaagacgaccacagctcagccgattgcgacgtctctccgtgagcgattggcccgactgacgtcctcaaagcagaaccaggtgctgctcggcctgattcggacaggcatctgcaccgttctcggccttcgtaatccggaaggcatcgaggaccaacgagccttccgcgacctcggcttcgactcgctgacgtcggctcagttcagcaaggaactcgccaaggaaaccggactgccactccccccgtccctggtcttcgactatcccaccccgcaggaatgtgctgcccatctgcgcacacaactcgtcgacctagacgacgaagaggacgcggcactgtcgaatgctctcccgcaagtggcccatcggcgtaccgtcgaggacgaaccgatcgccatcatcggtatggcatgtcgcttccccggcggcgtacgttctgccgacgacctgtgggaattgctcgcttcgggtaaggacgctatcggcgtcttcccgaccgaccgcggctgggacctggacacgctctacgaccccgaccccgaccaccccggcacctgctacacccgaaacggcggattcctctacggcgcaggccacttcgacgccgaattcttcggcatcagcccccgcgaagccctcgccatggacccccagcaacgactcctcctcgaaaccgcctgggaaaccatcgaacacgccggcatcaacccccacaccctccacggcacccccaccggagtcttcgccggaatcaacgctcaagaccacgccgcgcatatccgccaaagccgtgatgtggagaccatcgagggctacgccctgaccggcagttcgggaagtgtggcgtccggccgggtggcctacacgctcgggctcgaaggccccgcggtgtcggtggatacggcgtgttcgtcgtcgttggtggcgttgcattgggcggcgcaggcgttgcgtgcgggtgagtgttcgatggcgcttgccgggggtgtgacggtgatgtcgtctccgggtacgtttgtggagttctcacgtcagcggggtctggccgcggacgggcggtgcaaggcctattcggcggctgctgacggtaccggctgggccgagggtgtggggatgctgctggtggagcggctctccgacgcccgtcgcaacggtcaccgtgtcctggccgtggtgcgtggcagtgcggtcaaccaggacggtgcgagcaacggtctgaccgcgcccaacgggccctcccagcagcgtgtcatccgtcaggccctggccaatgcgggactgaccccggccgatgtcgacgcagtggagggccacggcaccgggaccactctgggggacccgatcgaggcccaggcactcctggccgcctacggacaacaccgcccccaccaccgccccttgtggctgggatccctcaaatccaacatcgggcacgcacaggccgccgcgggcgtgggcggagtcatcaagatggtgatggccctgcgcaacgggctgctgccacagaccctccacgtggacgagcccaccccccaggtcgactggtccacaggcgcagtacaactcctgacacaaccggtgccctggcccgccgacccggccggccggccacgccacgccggcgtgtcatcattcggcgtcagcggcaccaacgcccacatcatcctcgaagaagcacccactccccaggacagcgataccgacgacgaaccgcctgccaacgcaccagccctgccccatcccctccctcttcccgtgccggtgtcggcgaggtctgaggccgggttgcgggcgcaggcacaggcgttgcgccagtacgtggcagcccgcccggacatgtcacctgccgacattggtgcgggtctggcccgcggccgggccgtactggaacaccgcgccgtcatcctggccgcggaccgcgaggaactggcgcaggcactgacagccctggcagccggcgaaccccacccccacatcaccacaggccacacccggggcggtgaccgcggcggcgtcgtcttcgtcttccccggacagggcggccagtgggccgggatgggcctgaccctgctcacctcctcacccgtgttcgccgaacacatcgacgcatgcgagaaagccctcaccccctgggtgccctggtccctgaccgacatcctgcaccgcgaccccgacgaccccgcatggcaacaagccgacgtggtccagcccgtgctcttcagcatcatggtctccctcgccgccctgtggcgctcctacggcatcgaacccgacgcggtcctcggccactcccagggagaaatcgccgccgcccacatctgcggcgcactcagcctgaaagacgccgccaaaaccgttgcactgcgcagccgcgcactggccgccgtacgaggccggggcgccatggcctcactgcccctgcccgcccaggacgtgcagcagctcatttccgaacggtgggaagggcagttgtgggtggcagccctcaacggcccccactccaccaccgtctccggcgacaccaaggcggtggatgaggtgctggcgcactgcaccgacaccggcctacgggccaaacgcatccccgtcgactacgcctcccactgcccccacgtccaacccctccacgacgaactcctgcacctgctgggagacatcaccccccagccgtccaccgtgccgttcttctccaccgtggaaggcacctggctggacaccacaaccctggacgccgcctactggtaccgcaacctccaccagcccgtccgcttcagccacgccatccagaccctgaccgacgacggacaccgcgccttcatcgaaatcagcccccaccccaccctcgtccccgccatcgaagacaccaccgaaaacaccaccgaaaacatcaccgcgaccggcagcctccgccgcggcgacaacgacacccaccgcttcctcaccgccctcgcccacacccacaccaccggcatcggcacacccaccacctggcaccaccactacacccaaacccacccccaccccaacccccacacccacctcgacctgcccacctaccccttccaacaccagcactactggctccaaccacccaccacaacaaccgacctcaccaccaccggcctcacccccacccaccaccccctcctcaccgccacactcaccctcgccgacaacaacacacaactactcaccggccgcctctccctacgcacccacccctggctcaccgaccacaccgtcgccggcatggtcctcctgccgggcaccgcgctcctcgaactcgccctccaagccggcgaacgggtggactgccctcgggtggaggaactgaccctgcacgcaccgttggtgatcccgcacaccgaggacgtgacgttgcaggtcaccgttcgggcagccgatgagagtggccatcgcgccctcgcgatccactcgtactccggcaccgcgtcgtcggcggaccgggagtggacccgtcacgccacgggcctcctcacacaccacgccgacaccgatcaccgtgccgacacgcacacggacgcgtgccttggcgggagctggcccccgcccggcgcgcagcccatcgaactgggcgacgtctacggtcgtatggcggcggactcggacatcgcctacgggccggtcttccaggggctgcacgccgcctggaggttcggcgacgatgtcctggccgaggtgcgtctgccggaagaggctctgcgcgatgctccggcggcggccttcggtgttcacccggccttgctcgacgcggccctgcacgccacggcgctcaccccccagaacggggacggctcgacggagaacgtcgcccaggagagcatgcctgaccgcgcagcccaccaggcgcgactgccgttcagctggagcggcgtgtccctgcacacggcgggcagttccgtgttgcgcgtacggctgtcgcgcagtccgcagcacggtaatgccgtggccctcaccgcggccgacgaggacggtcggccggtggtgacgatcgagtcgctcgcgctgcggccggtgtccaccgaggagctgcgcgcggccgcggatcgtacgcccgagcacgagtcgctcttccgactggactgggtttccgtaccagtgcccgccaacgccccttcgcccaccgcggaccggccctgggcggtcatcggcgcgggccttccccacctgcccggcctgacggagcacgagcacgtgaccgcgtatgacgagccggcggacctgcttctggctctggaccgcggtgctccgccgcccggtgtgctggtcgtaggtggtgtcgcccacaccgaagcccgggagtattccgccgaagcccccggggagcgcgggaccgaggcctgcgaggcccggccggacgtcgtgcacgtgggcgtcgtgcacacggctgccgtgcacgcggctgccgcgcagatgttggccaggctccaggcctggctgggcgacgagcgcctcgcagacagccggctgctcgtcctgacgtgcggcgcggtcgcccgcgcctccggcgacgatgcgacggacctgcccggggccgccgtgtgggggctggtgcgttcggcgcagtccgagcacccggaccgcatcacgctgctggacttcgagcggggcacagaggcggagcccggtcagctggcgacggcgctgaactgcggggagcggcagcttgccgtccgccccggagggctgttcacgccacggctggtgcgcgcgccacgtgtcgccgacgccgtacccgccgtacccgccgtggccgtaccgtcagcgggtcacgcagccgtaccggcagcgggtcccttccttccgggcggaacggtgctgatcaccggcggaaccggtgtcctgggccggctcgtggcccggcatctggtggaggcgcacggcgtacggcatctgttgctggcgggtcggcgcggaccggacgccgagggtgcgccggagttgcgggcggagctcggtgggctcggcgcgacggtggaggtcgtcgcctgcgacgcggcggaccggcagcagctggccgacctgctgacacggatccccgacgatcggccgctgaccggtgtcgtgcacagtgcgggcatcctggacgacggcgtgatcacgtcgctgtcgccggagcggctcggggccgtcctccgggccaaggcggacgctgcgctgcttctcgacgagctgacgcgcggggcagagctgtcggctttcgtcatgttctcctccgcgtcggcggtggtcggctcgcccgggcagggcaactacgccgccgccaacgccgtcctcgacttccttgctcatcgccgccgcgccgaggggctgcccgccgtctctctcgcctggggcctgtgggaagagggcacagggatgacgggccacctcgacgtcgacgaccatgcgcggatcagccgcgcgggaatgcggccgctgccgactgccgaggctctggcgctgttcgacgcggccttggccgacggcgagccgttcctgatgccggctcggctcgacctcacggccgtacggtctggtgccgcgtccgcaccggtgccgccgctgctgcaaggtctgcttcagctgcctcggtcccgctcggccgccgcggcccccggccatggggccccggcggcggacgaggcggcggcctggcgtgagcgtctggcccggcagagtgccggtgagcgcaggcaggcgctgctgcgcctggtgcggtcgcatgtcgcggcggtgctcggccatagcggtgccgacggaatcgacgcatcgcgggcgttccgcgagctggggttcgactcgctcacggcggtcgagctgcgcaaccgtctcacggccgcgacgggcctgcggctgcgggccacgctggccttcgatttcccgaccccggcagcgctggccgagcacttgggcgagcgtctgcttcccgaccaggaggccacgggcgagcaagccggcgatcagctctccggcggcagcgaggaggacgtacgcagcctcctgacgtccattccgatcggcaggctgcgggacgcggggctcctcgggcccctgctcacgctcgcggacacgggccgcggcgcctcgggcgccgccgcaggtccggaggacgcgccgccctccggccaggacacaccggctcccgtctcgatcgacgagatggacatcgacgacctgatggatctggcgcacgggcatggcaccgcacccgcccgtgagcccgccgacgcagaggactcgtcgtcatcacgaaaccggacacaccacacacacgaaggtgagacagcgtga

SEQ ID NO: 2 - DNA, 31422 bp, Streptomyces avermitilis
  CDS (1)..(14643)
  CDS (14824)..(31419)
atggctaacgaggaaaagctccgcgactatctcaagcgcgttactgccgatctcctcaatgtgcggcgtcgacttcagcagattgaatcgggcgagcaggagccgattgcaattgtggggatggcgtgccgttttccggggggtgtggagtcggcggaggatttctgggagttgattgcgtcgggtcgggatgcggtgggggagtttccggtcgaccggggttgggacgtggaggctttctatgatccggagccggggcgggcgggttcgtcgtatacgcgccggggcggtttcctggagggtgcggcggagttcgatgcggggtttttcgggatcagtccgcgtgaggcgttggcgatggatccgcagcagcggttgatgctggaggtgtcctgggaggcgttggagcgggcgggcatcgaccccgccacgttgcgcggcagccggacgggcgtcttcgccggcctcatgtcccaggactacgcgacccgtctgctctcggtccccgacgacctggccggctacctgggcaacggcaacgcgggaagcatcctgtccggacgcgtcgcctacaccttcggcttcgagggccccgcggtgacggtcgacacggcgtgctcgtcgtcgctggtggcactgcacctcgcctgccagtcactgcgcaccggtgagtcctccttcgccctcgccggaggcgtgacggtcatgtccaccccgggcatgttcgtggagttctcgcggcagcggggtctgtcgccggacggccggtgcaaggcgtacgcgtcggctgccgacggcaccggcatgtccgagggcgtggggattttgctgctggagcggctgtccgaggctgaacgtcgtggtcatcgggttttggcggtggtgcgggggagtgcggtgaatcaggacggtgcgtcgaatgggttgacggcgccgaatggtccgtcgcagcagcgggtgattcggcaggcgttggcgtgtgcggggttgtctgtggcggatgtggatgtggtggaggggcacgggacgggcacgacgctgggtgatccgatcgaggcgcaggcgttgctcgccacgtacgggcagcgggccggtgacacgccggtgtggttggggtcggtgaagtcgaacatcgggcatgcgcaggctgctgcgggtgtggcgggtgtgatcaagatggtgatggcgttgcgggcgggggtgttgccgcggacgttgcatgtggatgagccgtcgtcgcaggtggattggtcgagtgggtcggttcgtgtgttggcggatgaggtggagtggccgggggtggagggtcggctgcggcgtgcgggggtgtctgcgttcggggtgagtgggacgaatgcgcatgtgattttggaggaggcgtcggggggcgcgggtgggggtgcgggccggctgcaggagttgggtccgggggtggtgtcgggttcgggggtggtgccgtgggtggtgtcggcgcggtcggagttggcgttgcgggggcaggcgcgtcggttgcgtggggttgtggcggttggtgggggtgcggatggtgtgggggtgagtccggctggggtcgggcgggctttggtgtcggagcggtcggtgttcgagcatcgtgcggtggtcgtggccgaggaccgcgacgagttcctgcacgcactcgacgcactggccggcggccgccccgtgcccggcgtcgtcgagggacgaaccacctcgggcgaactcgccgtactcttcgccgggcagggaacccagcgcgcaggcatgggccgcgaactgtacgaggcgtaccccgtcttcgcccaggccatcgacgagatctgcgcggaggccgacaccgcccgcaccgaccccggtgcccctgggctgcgggacgtactcttcgcaccgcaggactctcccgaaggccggctgatcgaggacacgggtttcgcccagcccgccctgttcgccttcgaggtggcgctgttccggctgctggagacctggggtctgacgcccgactacgtcctcggccattccgtcggtgaactggcggccgcccatgtcgccgggatgctctgccttgccgacgcggtggcactggtggtcgcacgaggccgcctgatgcaagggctcccgtccggcggagccatggtggccatcgaggcgtccgaggacgagatcctcccgctgcccgacgaatacgcatcccgggtcgcgcacgccgcggtgaacgggccgcggtcgatcgtcctctccggggacgaggacgcggtcctggacctcgcgcagcaatgggcggcacgaggccgccgcacccggcggctgcggaccagccacgccttccactcgccgcacatggacgccatgttgggcgacttccgccgcgcggccgagcaggtcaccttcagcgccccgcggattcccgtcgtctccaacgtcaccggcgcgcccctccccgccgagaccatgtgcaccccggactactgggtcgaacacgcccgcagcacggtccgtttcgcggacggcatctcatggcttcaggaacagggcgtcaccacctgcctcgaaatcggccccgacggcacgctgtcggccctcgcacaggactcgctcagtgcaccggcccgcgccatccccgccctgcggccggaccagccggaggcacggtcggtcatgaccgccctggcggagttgttcgtggctgggacggcggttgagtgggccggtgtgttcgaggggactgctcgcgaggtcggtgatggatgcggggtggagctgccgacgtatgcgtttgagcgggagcgattttggctggacgtggaggagggatctgcgggaggttccggggtttccgggatgtggggtggtccgttgtgggaggcggtcgagtgtggtgatgcgggggtggtggcatcgctccttggggtggatgagggggcgtcgctgggtgcggtggtgtcggcgttgggggaatgggggcgggtacggcacgagcgtgaagtggtggacgggtggcgctatcgggaggtgtggcgacccgtttcgggcggtggtgtaggggggctgtcgggcgcgtggctggtggtgtccgagggcgaggcgggcccggttgatgtggtggcggaggggttggagcggtgtggggcgcgagtggttcgggtggaggtggaagcggggtgtgtgagcagggaagtgttggccggccacctgcgtgaggcggtcgatggtgaggctgtcggcggtgtcgtctcccttgtgggctgggggagtggcgtcgtgcaggcgggagtggcgtctgtggggttggtgcaggcgctgggtgatgtgggcgtgggggcgcggctgtggtgtgtgacgggcggggccgtgtcggtggggggccgggatgctgtgtgggggccggcctcgggtgtggtgtgggggctgggccgtgtggtgggggcggaggcaccggaccgctggggtgggctggttgatgtgccggagctcgtggatgagcgggtggtcgatgggttggtaggtgtgctggcgggtgtggggggagggggtgagagtgagtttgccgtgcggtcttcgggggcgtttgtgcggcggttggtgcgggcgccgttggaggaggccgtcgcggagcgggagtggcggccccgcggcaccgtactcgtcaccggaggcaccggcgagttgggtgcgcacgtcgcccggtggatggcccggcgtggcgccgaacacctgctgctggtgagccgacgcggggagagcgcccagggagtcgaagaactccgagcggacttgatgggcttgggcgcgcgggtgtcggtggtggcgtgtgatgcggcggaccgtgaggcgttggcggaggtgttgcggtcggccgttccggcggagtgcccgctgggtgtggtggtgcatgccgcgggagttgtggatgacggggtgttggaggggttgtcgtccgagcgtgtcacgggggtgctgcgggcgaaggcgctggcggcctggaatctgcatgagttgacgcggggggcggatctttcggggttcgtggtgttctcgtcggctgcggcgacgttcgggccggcgggacaggggagttacgcggcggcgaacgcgtatgtggaggcaatcgttcggcaccggcgtggtgagggcctgccggggttggcggtggcgtggggtccgtgggctggtggggggatggcggagggggccgtggggcagatgcggcgtcggggtctggcggcgatgacgccggagacggcgctggtggcactgggccaggcgttggaccatgacgagacctgtgtgacggtcgccgacatcgactgggaccgattcaccgccaactccctccccggctcccgactctcgcccctcatcagcgacatccccgaagcacgcctcgcccgggaaaccaccggactcgacaccgccaccgcatcccccgactcgttctccgcacggctcaaggccatggacaccgccgagcaggaacgtgcgcttctcgacctggtccgtacgtacgcggcgaccgtgctcggacacagcacccccaccgccgtacgccctgagcgagccttccgcgacctgggcttcgtctccgtgagcgccgtcgaactgcgcaaccgcctcaacgccgtcaccgggctcctcctgcccaccacgctgatcttcgactaccccactccctccgcgctggccggatacctcaaggaacagctggaggagggcgcgggcggccagcgtgacattgctcctccggtcccggcgtcgcgtgtcgacgttgacgagccgattgcgattgtggggatggcgtgccgttttccggggggtgtggagtcggcggaggacttgtgggaactggtcgcgtcgggtcgggatgcggtgggagagtttccggtcgaccggggttgggacgtggaggctttctatgatccggagccggggcgggcgggttcgtcgtatacgcgccggggcggtttcctggagggtgcggcggagttcgatgcggggtttttcgggatcagtccgcgtgaggcgttggcgatggatccgcagcagcggttgatgctggaggtgtcctgggaggcgttggagcgggcgggcatcgaccccgccacgttgcgcgggtccacgaccggtgtcttcgccggcatgtgcagtcaggactacgccgacctcgtgcgccgggccaccgaggacctcgagggctacgccatgacgggcctgtccagcagcgtcacatccggacgcgtcgcctacaccctggggctcgagggtccggcggtgacggtggatacggcgtgttcgtcgtcgttggtggcgctgcatctggcgtgtcaggcgttgaggtcgggggagtgttcgctggcgttggcggggggtgtgacggtgatgtcgacgccgggtgcgtttgtggagttctcgcggcagcggggtctgtcgccggacggccggtgcaaggcgtacgggtcgggggccgatggggtcggctgggccgagggtgtgggtgtgctgttggtggagcggctgtccgaggctgaacgtcgtggtcatcgggttttggcggtggtgcgggggagtgcggtgaatcaggacggtgcgtcgaatgggttgacggcgccgaatggtccgtcgcagcagcgggtgattcggcaggcgttggcgtgtgcggggttgtccgtggcggatgtggatgtggtggaggggcacgggacgggtacgacgttgggtgatccgatcgaggcgcaggcgttgctcgccacttatgggcagggtcgttcgggggagcggccggtgtggttggggtcggtgaagtcgaacatcgggcatgcgcaggctgctgcgggtgtggcgggtgtgatcaagatggtgatggcgttgcgggcgggggtgttgccgcggacgttgcatgtggatgagccgtcgtcgcaggtggattggtcgagtgggtcggttcgtgtgttggcggatgaggtggagtggccgggggtggagggtcggctgcggcgtgcgggggtgtctgcgttcggggtgagtgggacgaatgcgcatgtgattttggaggaggcgtccgggggcgcggatgggggtgcgggccggctgcaggagttgggtccgggggtggtgtcgggttcgggggtggtgccgtgggtggtgtcggcgcggtcggagttggcgttgcgggggcaggcgcgtcggttgcgtggggttgtggcggttggtgggggtgcggatggtgtgggggtgagtccggctggggtcgggcgggctttggtgtcggagcggtcggtgttcgagcatcgtgcggtggtcgtggccgaggaccgcgacgagttcctgcacgcactcgacgcactggccgagggggcacccaccgcgggggtggtacagggtgtggccggaccggcggccgacggaaagatcgccatgctgttcggaggacagggcacccactgggaaggcatggcgcaggaactcctcggctcctcaccggtcttcgcccagcagatgtccgactgcgcccaagccctcgaaccgtacctggactggtctctcctcgacgtcctgcgcggcgcaccggacgcaccccctctgcaacgcgtcgatgtcgtccagcccgtcctcttcgcggtgatggtctcgctggcggcgctctggcgctcgtacggtgtacacccggacgcggtggccgggcactcgcagggcgagatcgcagcggcctacgtcgccggtgcactctccctcgacgacgccgcccgggtcaccgccctgcgcagccaggcgctggccgcactggccgggcagggggcgatggcatcggtcggtctgccggtcgagaagctggagccgcgtcttgcgacatggggcgaccgtctggtcatcgccgccgtgaacggggcgcgttcggccgtggtctccggggagccggaagcggtcgacgccctggtggaggagctgtcacacgaagacgtaccggcccgcaggctcatggtcgactgggcgtcgcactccccgcaggtcgaggcgatccaggggcggctgctcgaactcctcgcccccatccgcgcgaggaccggcgacgtgcccttctactccaccgtcaccggcgaacgcatcgacggcaccgaactcgacgccgactactggtaccgcaacctgcgccaggtcgtccgcttccgggacgcgacacaggcgctggtccgcgccggccacaccgtcttcatcgaggcgtgcccgcatccggccgtcgcggtcggtgtgcaggaaaccctggacgagatgggtgacttggacagcctggtcgtcggatctctgcgccggggcgaaggcggcttgcgacgcttcctgatgtccgtggccgagttgttcgtgggtggggtggcggttgagtggtccggtgtgttcgggagtgttggtcgcggggtcgctggtggttgcggggtggagctgccgacgtatgcgttcgagcgagagcgcttttggctggatgtggagggggcgccgcggggttccggggtctctgggcagtggggtggtcagttgtcggaggcggtggacaccgtgcgcggcggcatgctgcgcgactgcctcgccggactcgaccccgccgcacaggccgagaccgtgctggacctggtccttacccatgccgcggccgtccttggacacggcaccgccgatgcggtggtgcccgagcgcgccttccgcgacctcggtttcgactccctcaccgccgtcgaactacgcaaccgcctcaacaccgccacgggcctgcgcttcccgaggaccctggtgttcgaccatccccgcccggtggcactcgcggcacacatccacgagcagctgagcggcggaagcccgaccaccggcactgcccttgcccttgcccttcgggccccggcaccgcgtgtggatgtcgacgagccgattgccattgtggggatggcgtgccgttttccggggggtgtggagtcggcggaggatttctgggagttgatcgcgtcgggtcgggatgcggtgggggagtttccggtcgaccggggttgggacgtggaggctttctatgatccggagccggggcgggcgggtacgtcctacacgcggtgtggtgggtttttgcagggtgcggcggagttcgatgcggggtttttcgggatcagtccgcgtgaggcgttggcgatggatccgcagcagcggttgatgctggaggtgtcctgggaggcgttggagcgggcgggcatcgaccccgccacgctgcacgggtccacgaccggtgtcttcgccggcgtctcgcagcaggactacgccgagctcctgcgccgcggcacccaggaccacgaggggtacgcgctcaccggcgtctccaacagcgtcgtctccgggcggctttcctacaccttcggcttcgagggtccggcggtgacggtggatacggcgtgttcgtcgtcgttggtggcgctgcatctggcgtgtcaggcgttgaggtcgggggagtgttcgctggcgttggcggggggtgtgacggtgatgtcgacgccgggtgcgtttgtggagttctcgcggcagcggggtctgtcgccggacggccggtgcaaggcgtacgggtcgggggccgatggggtcggctgggccgagggtgtgggtgtgctgttggtggagcggctgtccgaggctgaacgtcgtggtcatcgggttttggcggtggtgcgggggagtgcggtgaatcaggacggtgcgtcgaatgggttgacggcgccgaatggtccgtcgcagcagcgggtgattcggcaggcgttggcgtgtgcggggttgtccgtggcggatgtggatgtggtggaggggcacgggacgggtacgacgttgggtgatccgatcgaggcgcaggcgttgctcgccacgtacgggcagggtcgttcgggggagcggccggtgtggttggggtcggtgaagtcgaacatcgggcatgcgcaggctgccgcgggtgtggccggtgtgatcaagatggtcatggccctgaaccacgaactgttgccgaccagcctgcacatcgacgaaccctccccccacatcgactggtcgagcggcggcgtccggcttctcaccgagcccgtaccgtggcagcagaacggccggcccaggcgcgcgggcgtctccgcgttcggagtcagcgggaccaacgcccacgtcatcatcgagcaggcgccggtcgaggcgcacgtcatcagtgagccggtaccggctgaggcgcacgtcatcgtcgagcaggcgccggtcgaggcgccccacgtggtcgacgccaccggaccggcggacctcaccgagccgcaagaggaggcggctgaaccggagtgcgtcgctgacgccgtgaccgagatgtcggctgaaccggagtgcgtcgccgacgccatgtccgagatgtcggctgagtgcgtcgccgaggccgtgtccgacaagtcggctgaaccggagtgcgtcgccgacgccatgtccgacaagccggccctcctgcccatcccgtggctgctctccgccaagtccgagcgagcgctgcggggccaggcgcgacggttgcggcagttcgctgccagggcatccgatgcccggccggccgacgtggcgcacgccctggcggcacagcggtccgtgttcgatcaccgggccgtcgtcgtggccgaggaccgcgacggcttccttcaggccctcgacgcgctggccgagggccggtcggcggacggcctgatcgaagggtcggtcggcccgcgtggcggccactcaggccgccggcgcggaaagaccgccatgctgttcgccggacagggcacgcaacgcgtgggaatgggccgtcagctgtatgcggctcacccggcctacgcggacgcgctggaccaggtactggcggaactggacggtcacctggaccagcccctgcgcccgctgatccacgccagtgcggatcttgcggatgtcgcggatgccgcggatgttctggaccgtacgcggtacgcccagccggcgctgttcgccgtccaggtcgcgctcttccggcacctggaacgtctcggcgtgcgcgcggacttcgtggccgggcactcgatcggcgagctcgcggccgcccacgtcgccggggtgcttcccctggcagcagcctgccgcctggtggcggcccgcgggcgcctgatggagcagctcgcaccaggcggcgccatggtcgccgtacgggcgagcgaagccgaggcgcgacaggcgctcgacggccgggaagcccgggtgtcggtcgcggccgtgaacggacccgcctcggtggtgttctccggcgccgaggacgaggtggggaacatggcggactggttcgccgagcgcgggcggagagtcaagcgcctgcgaaccgggcatgccttccactcaccgctgatggacccgatgctggaggagttccagcaggtcgcggcctcgctgacctacagcgaaccagccattcccatggtgtcgacgctcaccggcgacatcgtggcggcgggagaactgagcgaccccgagtactgggtccggcaggtacggcggaccgtgcgcttcggcgacgcgatcagccgcctgcacaccgacggagtccgcaccttcatggaactgggcccagacgggaccctgtcggcactggccgaggaatgcctagaggccaccgccgacagccaccccgccgacgacgacaccggcaccccgcaagagaacctgctcatcccgctcctacggccggacagcccggaacccggcaccctgctcaccggcttggcccggctgcatacgcacggagcggcggcggtcaactggcccgccgccctgcccgaacgcgatcgagcccgccacctcgacctgccgacctacgccttcgatcaccaccgctactgggtcgacacctcggccggccacccgggggacctgtcggcagcggggctcggcaccgccgggcatcccctgctcggttccgcggtggcactggccgagtcgcaggaactcctcttcaccggccgtctctccctgcgcacacacccgtggctggccgaccacgccatcttcggtaccgtcctgctgcccggcacggccatcctggaactggccgtgcgcgcaggcgacgaggtcgactgcggcaccgtcgaggaactcaccctgcggacaccgctcgtccttcccgaacagggctcggtgatcctgcaactctccgtcggggcaccccagggcccccagacgcccgaggagcccgaacggcgcaccttcgccctgtacgcccgcgaagacgacggactgtcgtcctcgtccgcggcggcgaccggcaccgagtggacctgccacgccaccggcgtcctgaccggcaccgcccggcccgcggaggagcacacacaggaaccgtggccgcccgccgacgcagcaccggtggacctggacggctggtacgagcagctggccggcgccggcctgggatacgggccggtgttccaggggctgcgcgaggtctggcggcgcggggacgaggtgttcgccgtcgtcaccctgcccgagagcacggagggacaggcggccgacgccgcccggtacgccctgcacccggccctgctggacgcggcactgcacccggtcgttctgcgccacgagggcgatgccgccgccgacggacacggctggctgccgttctcctggaccggcgtcacggtcgccgcctccggcgcctccaccctgcacgtccgtctcaccgtccgcacggacgaggacgcggtcggactgctggccaccgacgcatcgggacgcatcgtcatctccgcggggtccctcgccttccggcccgtctccgccgagcagctccaggccgcgcgcaccggctaccacgaccacctcttccgcatcgaatggcggccgctgcacctccccaccacaccggcacggacagccgactgggccctaatcggccccggtgcccggcggacggccgccgtcctggagcgcaacggcgcctcctggcaggcctacccggacccggcggctctcgcagaagccctggcggccggcgccccggcaccgggcatggtcgtcatctcgtgcgagccggacggcgcatccgcccccaccgattccgccctcaccgattccgccctcaccgattccgccccggccggctcggccccggccgactccaccgccctcgccgacgccacccggcaagccaccacccgcgtcctcgccctgctccaggaatgggtcgccgacgaacggctcgcggcctgccgcctggccctcctcacgcacggctcggtcaccgcgacccccgacgagcccgtgtccgacctcgcacacgccgccgtctggggactggtccgctccgtgcagaccgagaaccccgaccggttcctgctggccgacaccgacgacaccgacgcctcccgcaacgcccttcccctgctggccggggaaccgcagatcgccctgcgaaatggtgccgtccgcatcccgcggatgacacgagtgcccgtccggcagccacagccgagcaccaccgacgccgactgggacccggaggccacggtcctcatcacgggcggtaccggcgtcctcggccggctcgtcgcccgtcatctcgccacggcccacggggtacggcacctgctgctggccacccgccgcggcacggccgcggacggcgccgccgacctggtcgccgaactcgccggcctcggcgccgaggccacggtcgcggcctgcgacatcggggaccgggcggccgtcgccgcgctcctcgaccaagtgcccgcgcagcaccccctgaaagccgtgatccacacggccggtgtggtcgacgacggcatcctcacctcgctcactccggagcgcatggaggccgtcctgcacgcgaaggcgttcggcgccgcgcacctgcacgacctgacccgcgacgccggcctcaccaccttcaccgtcttctcctcggccgccgcctccttcggcagtcccggacagggcaactacaccgcggcgaacgcctttctggacgccctgatgcagcaccgccacacccaggcactgccgggccggtcgctcgcctggggcctttggggcgaggccgacggcatgacccgcaacctcgccggcaccgacttcgcgcgcatggcccgcggcggcctgctccccctgtccaacgcacagggactcgcgctcctcgacacagcggatcgcctcggccctttcggtgacgggctgctcctcgccacccggctcgacgcggccaccctccacgcacaggccacggccggcgccctgccgcgcatcctgcacgggctgatccgcatcccggcccggcggtccgccgaccacggcatcgcgaccgacacccccgccacgctgcgcgagcgcctggccggactcaccatccccgcgcagcgcaccggtctcctcctggaactcgtacggacccatgccgccgccgtcctcggccaccccaccagcgccgtcacagccgcggacggcgcactcccggacgatctggtcccggccgacaccgagttccgcgacctcggcttcgactcgctgaccgccgtcgaactccgcaaccggatcaacgccgtcaccggcctgcgcctcccggcaacgctcatcttcgaccagcccagccccgcggcactcgccgatcacctcgcgacccgcctgacggccgaggcgggcacgccggacgagccggcccctgccgccgcggcagccggggccgggagcgcagggagtgccgagaccggacagcagcgcagtacggggagcgagaagcagcagaccaggggcggcacctccaccgaaaccgtcgaatccctgttctggatcggacacgacacccgccgcatcgaggagtccatggccctgctctcggcggcctccttcttccggcccgccttcacggacccctcggacatcccggagccgacgttcgtccggctcgcccagggtgaagcgcgcgcccaaggtgaagcactcgcccggggcgaaacacggcccgccctcatctgcctgcccaccgtcgccgccgtgtcgagcgtgtaccagtactcacgtttcgcggcgggactgaacggacaccgagacgtctggtacgttcctgcgccagggttcctggagggcgaacccctgccgtccggaatcggcgcggtgacccgcatgttcgccgacgcgatcgtccggttcaccgacggcgcgccttttgcgctcgccgggcattccgcgggcggatggttcgtctacgcggtgacgagtcatctggagcgtctaggcgtccgtccggaagcggtggtgaccatggacgcctatctcccggacgacggcatcgcacctgtcgcgtccgcgctgacaagtgaaatcttcgaccgcgtcacgcagtttgtggacgtggactacacacgcctggtcgccatgggcggatacttccgcatcttctccggctggagtcctccggacatcaccacacccgccctcttcctgcgcggccgggacggagaacagatgccgccgccgtggggagttccgcacaccgttctggacatccaggggaatcacttcacgatgctggaacagtttgcggattcgactgctcggcatgtcgacgaatggctgacagaaatcgcatcagtgcggcgctgatcgcgcctctgatcgcggtcctgatcgcggccctgatcggcgggtcgggcacagcccggtcggccggtcggccagtcggccagtcggtggtatccggtcggctccggcatcgatcagtgctttcccccttacggccatacgggcctttctgagacttcttgaatttgggagacagtgatggacacgtccagcgaaaagctcgtcgacgcgcttagggcgtctctgaaggcgaaccagaccctgcgggcacgtaatgagcaactggcagccgccatggaggcgtccagcgagccgattgcgattgtggggatggcgtgtcgttttccgggtggggtgtgttcgccggaggagttgtgggagctggttgcgtcgggtggggatgcgattggtgaatttccggccggtcgggggtgggatctggaggggttgtttgattcggaccctgaccggtcggggacgtcgtacgcgcggtatggcgggtttttgtatgaggcgggggagttcgatgcggacttcttcgggatcagtccgcgtgaggcgttggcgatggatccgcagcagcggttgttgctggagacgtcgtgggaggcgttcgagcgggcgggtatcgatccgctgtcgatgcgtggctcccgtacgggtgtcttcgccggggtgatgtaccacgactacggatcccgcctgggtaccatccccgagggattcgagggctacatcggcaacggtagcggcggcgccgtcgcgtcgggccgcgtcgcctacacgctcggtctcgagggccctgccgtctcggtggacacggcatgttcgtcgtcgttggtggcgctgcatctggcgtgccagtcgctgcggtcgggtgagtgcacgctcgcgctggccggcggtgtgacggtgatgtcgaccccgcacctcttcgtcgagttctcacgccagcgcggactgtcggtggacggccgctgcaagtccttcgcgggtggagccgacggcaccggcatgggcgagggcgtcgggatgctgttggtggagcggttgtcggatgcggtgcggctggggcatcgggtgctggcggtgctgcgcggcagtgcggtcaatcaggacggtgcgtcgaatgggttgacggcgccgaatggtccggctcaggagcgggtgatccggcaggcgttggcgaacgcggggttgtccgtggcggatgtggatgtggtggaggggcatgggacgggcacgacgctgggtgatccgatcgaggcgcaggcgttgctcgccacgtacgggcagcgggccggtaacaggccgctgtggctgggatcggtgaagtcgaacatcggccatgcgcaggctgccgcgggtgtgggtggggtcatcaagatggtgatggcgttgcgggagggggtgttgccgcggacgttgcatgtggatgagccgtcgccgcaggtggactggtccgcgggggcggtgcggctgctgacggaggcggtgccgtggccgggggacgcggcagggcggttgcggcgggcgggagtgtcgtcgttcggggtcagtggcacgaatgcgcatgtgattttggaggaggcgccggcggcggggggctgtgttgccgggggtggggtgttggagggtgctccgggtcttgccatttcggtggctgagtcggtggccgctccagtggctgtgtctgcgccggtggctgagtcggtgccggtgccggtgccggtgccggttcctgtgccggtgtcggctaggtctgaggctgggttgcgggcgcaggcggaggcgttgcgtcagtacgtggcagtccggccggacgtttcgcttgccgatgtgggtgcgggtctggcctgtgggcgggctgtgctggagcatcgtgcggtcgtcctggccgcggaccgtgaggagctggtgcaagggttgggggcgctggcggcgggtgagccggatcggcgggtgaccacgggtcatgcgccgggtggtgaccggggcggtgtcgtcttcgtgtttcccggacagggtgggcagtgggccgggatgggtgtgcgtctgctcgcctcctctccggtgttcgcccggcggatgcaggcgtgcgaggaggctctggcgccgtgggtggactggtctgtggtggacatcctgcgccgggacgcgggggatgcggtgtgggagcgggccgatgtggtccagcctgtgctgttcagcgtcatggtgtctttggctgctctgtggcgttcctacggtatcgaacccgacgcggtccttggccattcccagggcgagatcgcggccgcgcatgtgtgtggggcgctgagcctgaaggacgcggcgaagactgttgcgctgcgcagccgggcgctggccgctgtgcggggccggggcggcatggcctcagtgccgctgcctgcccaggaggtggagcagctcattggtgagcggtgggcggggcggttgtgggtggcggcggtcaacggcccccgctccaccgccgtctcgggggatgccgaggcggtggacgaggtgctggcgtactgtgccggcaccggggtgcgggcccggcggatcccggtcgactatgcctcgcactgcccccatgtgcagcccctgcgggaggagttgctggagctgctgggggacatcagcccgcagccgtccggcgtgccgttcttctccacggtggagggcacctggctggacaccacaaccctggacgccgcctactggtaccgcaacctgcaccagcctgtccgtttcagcgatgccgtccaggccctggcggatgacggacaccgcgtcttcgtcgaagtcagcccccaccccaccctcgtccccgccatcgaagacaccaccgaagacaccgccgaagacgtcaccgcgatcggcagcctccgccgcggcgacaacgacacccgccgcttcctcaccgccctcgcccacacccacaccaccggcatcggcacacccaccacctggcaccaccactacacccaccaccacacccacccccacaaccaccacctcgacctccccacttatcccttccaacgccagcactactggctcgacgctcccacgggagcaggtgacgtcgccgctgctggcttggagccggccgaacaccctctgctcgcggcaacagtccaactcgcagacacggacggctgcctactgacgggtcgcctgtccttgcgctcgcatccgtggctgggcgattacgaggtggggggtgcggtcctgctgtcggggtcggcgttcgtggagctggcggtccaggttggcgaacgcgtgggctgcacccgaatcgagcaactcactgtgcatgcgccgctggtggttcctgtgggtgggggtgtgagtgtgcaggttggggttgcggctgcggatggggaggggcggcgtttggtgagtgtgtatgcgcggggtgggagtgcttgtggtgggggtggtgcgtcgggtggggtgtggacgtgtcatgcctcgggggtgctggttgaggctgctgctggtggtggtgtggtggtggatggtctggcgggggtgtggccgccgcggggtgcggtggcggtggatgtcgatggtgtccgtgaccgtttggctggggctggttgtgttttggggccggtgttttcggggctgcgtgcggtgtggcgtgatgggggggatttgctggctgaggtgtgtctgccggaggaggcgtggggtgatgcggctggttttgggctgcatccggcgttgctggatggtgtggtccagccgttgtcggtgttgcttccgggtgggacggggtttggggagggggcggggttcggggagggtgttcgggtgccggctgtgtggggtggtgtgtcgcttcaccgggcgggtgtgaccggtgtgcgggtgcgtgtgtgggctgtagggcggggcggcgggcgtgaggcggtgtcggtcgtggtcggggatgaggcgggtgtgccggtggcgtcggtcgatcgtcttgagttgcggcctgtggatatgggtcagttgcgtgctgtctcggtttcggcggggcggcggggttcgctgtatgcggtgcagtgggctgaggtgggtcctgtgccggtgtgtgggcaggcgtgggcgtggcacgaggacgtgggtgagagcggtggtgggcctgtgccgggggtggtggtgttgcggtgcccggatgccggtgccggtggcggcggtggcggtggtgtgggtgaggttgttggtggggtgttgggtgtggtgcaggggtggctggggctggagcggtttgcgggttcgcggctggtggtggtgacccggggtgcggtggtggccggccaagaagacggcccggtggatgtggtgggtgcggcggtgtgggggctggtgcggtcggcgcaggctgagcatccggaccggtttgtcctcctcgacctcgacaccgacaccgacaccggcaccgacctcgacaccggtgctggtgctggtgctggtgctggttggggcgtggatggtgggcatgtggcggcggtggtggcgtgtggtgagccgcagttggcggtgcgtggtgagcgggtgctggccgcacgcctgacgcgacttgagtcgtccgttgatgtacctgctcagcggtccggtgatgttgctggtcgggaggtgttgccgtggttgtcgggtgggtcggtgttggtgacgggtgggacgggtgtgctgggtgcggcggtggcgcggcatctggctggtgtgtgtggggtgcgggatctgctgttggtgagccggcgtggtccggatgctccgggtgcggagggtttgcgggcggagctggccgcgttgggggcggaggtgcggattgttgcgtgtgatgtgggggagcggcgggaggtggtccggctgctggagggtgttcctgccgggtgtccgctgacgggtgtcgtgcatgcggctggtgtgctggacgatgcgacgatcgcctctctcacgcccgagcggctgggcacggtgttcgcggccaaggtggatgccgctcttttgctggatgagctgacgcggggtatggagctgtcggcgttcgtgctgttctcctcggccgcggggatcctggggtcggccgggcagggcaactacgccgcggccaatgccgctctggacgcgctggcgtaccggcggcgggcggcgggtctgccgggggtgtcgctggcgtgggggctgtgggaagaggccagcgggatgaccgggcacctggccggcaccgaccaccggcgcatcatccgttccggtctgcatcccatgtcgaccccggacgcactggctctcttcgatgcggccctggctctggaccggccggtcctgctgcccgccgacctgcgtcccgccccgcccctgccgcccctgctgcaggacctcctgcccgccacccgccgccgcaccacccgcaccaccactaccggtggtgcggacaacggcgcccagctgcatgcccggctggccggccagacacacgaacaacagcacaccaccctcctcgccctggtccgctcccacatcgccaccgtcctcggccacaccacccccgacaccatcccccccgaccgcgcgttccgcgacctcggcttcgactccctcaccgccgtcgaactacgcaaccggctctcccgcaccaccggactccgcctccccaccaccctcgccttcgaccaccccaaccccaccaccctcacccaccacctccacacacaacttctgggctcggacagcactgcctccatcccagctccccgtgctgcggctgtgcctgcagaccaggacgagcccgtcgcgatcattggcatggcgtgccgctatcccggaggcgtcacctcagccgaggagctgtgggaactgctcgcatcggggagggacacggtcggcgagtttccgacggaccgtgggtgggacctggaagcactgttcgatccggaaccgggtcggccgggcacctcgtacacccgctgtgggagtttcctctacgacgcgggggagttcgacgccggcttcttcgggatcagtccgcgtgaggcactggcgatggacccgcagcagcgattgctgctggaggcctcatgggaggccatggagcaggcaggtattgaccctacgaccgtacgcgggagccagacaggcgtgttcgcgggcctcattccgcaggcctatggacccaggctgcacgaaaacgccgcagccgacaccgagggctatgtcctgaccggcacatccgggagtgtggcctccggtcgtatctcgtacacgtttggttttgagggtcctgcggtgtcggtggacacggcttgttcctcgtcgttggtggctttacatctggcctgtcaggcgttgcgtgcgggtgagtgctcgatggcgcttgccgggggtgtgacggtgatgtcgtctccgggtgccttcgtggagttttcgcggcagcggggtctggccgcggacgggcattgcaaggcgttctcggcggcggcggacgggaccggctggggtgagggtgtggggatgctgctggtggagcggctctccgacgcccgtcgcaacggtcaccgtgtcctggccgtggtgcgtggcagtgcggtcaaccaggacggtgcgagcaacgggctgaccgcgcccaacgggccctcccagcagcgtgtcatccgccaggccctcgccaacgccggcttgtcggccggtgatgtcgatgcggtggaggcccacggcaccggcaccactttgggcgacccgatcgaggcccaggccctccttgcgacctacgggcaggaccgtgccggcgaggggccgctgtggctgggctcggtcaagtccaatgtcggtcacacacaggctgccgcgggcgtcgccggggtgatcaagatggtgatggcgctgcggaatggtctgctgccgcggacgttgcatgtggatgagccgtcgccgcatgtggactggtccgcgggtgcggtgcagctgctgacggagacggtgccctggcccggcggggaggggcggctacggcgggcaggagtgtcatcattcggcgtcagcggcaccaacgcccacgtcatcctcgaagaagcacccgcccacaacatcccgtcagacacacccgccgacgacgttccggggggaccacccgccggcgaggatgccggtagtggcgaggaggctgctgccggcagtccaggggtgtggccgtggctggtgtcggccaagtcgcagccggccctgcgcgcccaggcccaggccctgcacgcccacctcaccgaccaccccggcctcgacctcgccgacgtcggatacaccctcgcccacgcccgcgccgtgttcgaccaccgcgccaccctcatcgccgccgaccgcgacaccttcctgcaagcactccaggcactcgccgcaggcgaaccccaccccgccgtcatccacagcagcgccccaggcgggaccgggaccggggaggccgcaggaaagaccgcattcatctgctccggacagggcacccaacgccccggcatggcccacggcctctaccacacccaccccgtcttcgccgccgcactcaacgacatctgcacccacctcgacccccacctcgaccaccccctcctccccctcctcacccaggaccccaacacccaggacaccaccaccctcgaagaagcggccgcactgctccagcagaccccgtacgcccagcccgccctcttcgccttccaggtcgccctccaccgcctcctcaccgacggctaccacatcaccccccactactacgccggacactccctcggcgaaatcaccgccgcccacctcgccggcatcctcaccctcaccgacgccaccaccctcatcacccaacgcgccaccctcatgcaaaccatgccccccggcaccatgaccaccctccacaccaccccccaccacatcacccaccacatcaccgcccacgaaaacgacctcgccatcgccgccatcaacacccccacctccctcgtcatcagcggcaccccccacaccgtccaacacatcaccaccctctgccaacaacaaggcatcaaaaccaaaaccctccccaccaaccacgccttccactccccccacaccaaccccatcctcaaccaactccaccagcacacccaaaccctcacctaccacccaccccacacccccctcatcaccgccaacaccccacccgaccaactcctcaccccccactactggacccaacaagcccgcaacaccgtcgacatagccaccaccacccaaaccctccaccaacacggcgtcaccacctacatcgaactcggacccgacaacaccctcaccaccctcacccaccacaacctccccaacacccccaccaccaccctcaccctcacccacccccaccaccacccccaaacccacctcctcaccaacctcgccaaaaccaccaccacctggcacccccaccactacacccaccaccacaaccaaccccacacccacacccacctcgacctccccacctaccccttccaacaccaccactactggctcgaaagcacacagcccggtgccggcaacgtgtcagcagccggactcgaccccaccgaacaccccctactcggcgccacattggaactggccgaaggggacggctgcctactgacggggcgcctctcgttgcgcacgcatccctggctcgccggccatgcggtaggcggtgtcgtgctgctgccgggtacggccttcgcggaactggcccttcatgccggagaaagtgtgggttgcgaccacgtggacgagctgacgctccacacaccgttggtcattcctgaggtcggagacgtgacccttcaggttgccattgcggcgccggacgagtcgggtcgccgcatgatgaccatccactcacgcggtgagggcggcagtggtggagccgatgcgtcggccagtgcgtggacgcgtcatgccgcgggtgtgctgagccctgccaaggacgatgacactgcctcgtacgagctgcttgcgggaccctggcctcccgttggagctacgcctgtcgacctgaacacggcttacgatcaaatggccgacgccggctttgcttatggcctggcattccaagggttgcgcgcggcctggcgctacggcgacgacatcctcgtcgaggcacgtcttcccgaagaagtgtcgggagacgcggcggcgtacggtctgcacccggccctgctcgacgctgcccttcagggcaccggcctgctttctgtggcgggtccggggacgcccgtcgtgccccatgtgtggaacggtctgcggttccgtacgcatggtgcagtctccgtgcgcgcgtgcctgtcgacgcttggagcgacaggggcggccgtgtgcgtgcgcatcaccgacgacaccggggtgccggtggcgtcggtcgatcgtcttgagttgcggcctgtggatatgggtcagttgcgtgctgtctcggtttcggcggggcggcggggttcgctgtatgcggtgcagtgggctgaggtgggtcctgtgccggtgtgtgggcaggcgtgggcgtggcacgaggacgtgggtgagagcggtggtgggcctgtgccgggggtggtggtgttgcggtgcccggatgccggtgccgatggcggcggtggcggtggtgtgggtgaggttgttggtggggtgttgggtgtggtgcaggggtggctggggctggagcggtttgcgggttcgcggctggtggtggtgacccggggtgcggtggtggccggcccggaggacggcccggtggatgtggtgggtgcggcggtgtgggggctggtgcggtcggcgcaggctgagcatccggaccggtttgtcctcctcgacctggacaccgacctcgacagcggcgctgacgccgatgccggcaacgaggccggtatggggtctggtctggatggtgggcgtgtggctgcggtggtggcgtgtggtgagccgcagttggcggtgcgtggtgagcgggtgctggccgcacgcctgacacgacttgagtcgccggttgatgtatcgggtcgggaggtgttgccgtggttgtcgggtgggtcggtgttggtgacgggtgggacgggtgtgctgggtgcggcggtggcgcggcatctggctggtgtgtgtggggtgcgggatctgttgttggtgagccggcgtggtccggatgctccgggtgcggagggtttgcgggcggagctggccgcgttgggggcggaggtgcggattgttgcgtgtgatgtgggggagcggcgggaggtggtccggctgctggagggtgttcctgccgggtgtccgctgacgggtgtcgtgcatgcggctggtgtgctggacgatgcgacgatcgcctctctcacgcccgagcggctgggcacggtgttcgcggccaaggtggatgccgctcttttgctggatgagctgacgcggggtatggagctgtcggcgttcgtgctgttctcctcggccgcggggatcctggggtcggccgggcagggcaactacgccgcggccaatgccgctctggacgcgctggcgtaccggcggcgggcggcgggtctgccgggggtgtcgctggcgtgggggctgtgggaagaggccagcgggatgaccgggcacctggccggcaccgaccaccggcgcatcatccgttccggtctgcatcccatgtcgaccccggacgcactggctctcttcgatgcggccctggctctggaccggccggtcctgctgcccgccgacctgcgtcccgccccgcccctgccgcccctgctgcaggacctcctgcccgccacccgccgccgcaccacccgcaccaccactaccggtggtgcggacaacggcgcccagctgcatgcccggctggccggccagacacacgaacaacagcacaccaccctcctcgccctggtccgctcccacatcgccaccgtcctcggccacaacgcgccggagatgatccccgttgactcggcgttccgcgacctaggcttcgactccttgacagcggtggaactccgtaaccgcctgggtgaggcaacgggactgcgactgccgaccagtctggtcttcgaccagccgaatgcagcgaccctggcgcgtcacctacgtcgtgagctgatgggcgacgacgcggaaggcgagacgccatcgcaggtcgcacttcatcaggttgccgcggatgagccgattgcgattgtggggatggcgtgtcgttttccgggtggggtgtgttcgccggaggagttgtgggagctggttgcgtcgggtggggatgcgattggtgaatttccggccggtcgggggtgggatctggaggggttgtttgattcggaccctgaccggtcggggacgtcgtacgcgcggtatggcgggtttttgtatgaggcgggggagttcgatgcggacttcttcgggatcagtccgcgtgaggcgttggcgatggatccgcagcagcggttgttgctggagacgtcgtgggaggcgttcgagcgggcgggtatcgatccgctgtcgatgcgtggctcccgtacgggtgtcttcgccggggtgatgtaccacgactacgccgcgcgtctccaccatgtccccgagggtttcgaaggcctcatcgccaacggcagcgcaggcagcgtcgcgaccggccgggtggcctacagctttggccttgagggtccggccgtgaccgtcgatacggcgtgttcgtcgtcgttggtggcgttgcattgggcggcgcaggcgttgcgtgcgggtgagtgttcgatggcgcttgccgggggtgtgacggtgatgtcgtctccgggtacgtttgtggagttctcacgtcagcggggtctggccgcggacgggcggtgcaaggcctattcggcggctgctgacggtaccggctgggccgagggtgtggggatgctgctggtggagcggctctccgacgcccgtcgcaacggtcaccgtgtcctggccgtggtgcgtggcagtgcggtcaaccaggacggtgcgagcaacggtctgaccgcgcccaacgggccctcccagcagcgtgtcatccgtcaggccctggccaatgcgggactgaccccggccgatgtcgacgcagtggagggccacggcaccgggaccactctgggggacccgatcgaggcccaggcactcctggccgcctacggacaacaccgcccccaccaccgccccttgtggctgggatccctcaaatccaacatcgggcacgcacaggccgccgcgggcgtgggcggagtcatcaagatggtgatggccctgcgcaacgggctgctgccacagaccctccacgtggacgagcccaccccccaggtcgactggtccacaggcgcagtacaactcctgacacaaccggtgccctggcccgccgacccggccggccggccacgccacgccggcgtgtcatcattcggcgtcagcggcaccaacgcccatgtgattttggaggaggcgcctgcggcggcgggcggtgctgccggtggtggggtgtcggtgggtgctccgaatccagcccttccggtggctgagtctgagccggtgccggtgccggtgccggtgtcggcgaggtctgaggccgggttgcgggcgcaggcacaggcgttgcgccagtacgtggcagcccgcccggacatgtcacctgccgacatcggtgcgggtctggcccgcggccgggccgtactggaacaccgcgccgtcatcctggccgcggaccgcgaggaactggcgcaggcactgacagccctggcagccggcgaaccccacccccacatcaccacaggccacacccggggcagtgaccgcggcggcgtcgtcttcgtcttccccggacagggcggccagtgggccgggatgggcctgaccctgctcacctcctcacccgtgttcgccgaacacatcgacgcatgcgagaaagccctcaccccctgggtgccctggtccctgaccgacatcctgcaccgcgaccccgacgaccccgcatggcaacaagccgacgtggtccagcccgtgctcttcagcatcatggtctccctcgccgccctgtggcgctcctacggcatcgaacccgacgcggtcctcggccactcccagggagaaatcgccgccgcccacatctgcggcgcactcagcctgaaagacgccgccaaaaccgttgcactgcgcagccaggcactggccgccgtacgaggccggggcgccatggtctcactgcccctgcccgcccaggacgtgcagcagctcatttccgaacggtgggaagggcagttgtgggtggcagccctcaacggcccccactccaccaccgtctccggcgacaccaccgcagtagaagaactcctcacccactgtgccgacaccggcctacgggccaaacgcatccccgtcgactacgcctcccactgcccccacgtccaacccctccacgacgaactcctgcacctgctgggagacatcaccccccagccgtccaccatgccgttcttctccaccgtcgtagggcacctggtctggtacaccacaaccctggacgccgcctactggtaccgcaacctccaccagcccgtccgcttcagccacgccatccagaccctgaccgacgacggacaccgccccttcatcgaaatcagtccccaccccaccctcgtccccgccatcgaagacaccaccgaaaacaccaccgaaaacatcaccgcgaccggcagcctccgccgcggcgacaacgacacccaccgcttcctcaccgccctcgcccacacccacaccaccggcattcggacacccaccacctggcaccaccactacacccaaacccacccccacccccacaaccaccacctcgacctgcccacctaccccttccaacaccagcactactggctccaaccacccaccacgacaaccgacctcaccaccaccggcctcacccccacccaccaccccctcctcaccgcaacactcaccctcgccaacaacaacacacaactactcaccggccgcctctccctacgcacccacccctggctcaccgaccacaccgtcgtcggtaccactcttgtgccaggaaccgccctcctcgaactcgccctccaagcaaccacgaccgaccacctcgaagaactcgccctccacacgcctctcgtcatcccccgtgagggtgccgtcgacgttcaggtgcacatcaatccaccggacgacaccgacactcgttcactgacgatctactcgcgaagcgagaacgcccccgcagcggctccctggcgtcatcacgccacggccgttctgggaaccaagacctcgcgcattgagacaggccgtagccacgatgatctgtcgatgtggccgccagcgggcgcagttcgctgtgctgatgaggaattggcagccttgtatggcgactacgaggcaaatggctttgtctatggccccgcattccgggggctgactgctgcctggcgtctgggagacgaggtgtttgccgaggttcgccttccagaacaggtgcacggcgaggcatccgcgtacaacctgcacccggcactgctggatgctgccttgcacgcagcggcctttgcgccgtcgggcagtctgccgcagggatccgtaccgttctccttcaccggtgtgacgctgcacgccgccaatgcgtcgtcgttgcgcgtgcgactctcgccggccgatccgaacagcggccacgccgcagtttccgtgctggtcacggatgacaccggtacgcccgtggcgtccgtcgaggcgttggcggtgcgcccgttggcggcggacgaattgcgagctgccgagcgcgccgtacagcgcgctgagctcttcgacatgaagtgggttgaggtgccctcagatgtactggtgtcgggcggggcatcggtggtggtgctggatggtgccgacgacctcgttggtctggcggctgaggaggatggtgtgccgggggtggtggtgttgcggtgcccggatgccggtgccgatggcggcggtggtggcggtggtgtgggtgaggttgttggtggggtgttgggtgtggtgcaggggtggctggggctggagcggtttgcgggttcgcggctggtggtggtgacccggggtgcggtggtggccggcccggaggacggcccggtggatggcccggtggatgtggtgggtgcggcggtgtgggggctggtgcggtcggcgcaggctgagcatccggaccggtttgtcctcctcgacctggacaccgacctcgacagcggcgctgaccgcgatgccggcaacgaggccggtatggggtctggtctggatggtgggcgtgtggctgcggtggtggcgtgtggtgagccgcagttggcggtgcgtggtgagcgggtgctggccgcacgcctgacacgacttgagtcgccggttgatgtatcgggtcgggaggtgttgccgtggttgtcgggtgggtcggtgttggtgacgggtgggacgggtgtgctgggtgcggcggtggcgcggcatctggctggtgtgtgtggggtgcgggatctgttgttggtgagccggcgtggtccggatgctccgggtgcggagggtttgcgggcggagctggccgcgttgggggcggaggtgcggattgttgcgtgtgatgtgggggagcggcgggaggtggtccggctgctggagggtgttcctgccgggtgtccgctgacgggtgtcgtgcatgcggctggtgtgctggacgatgcgacgatcgcctctctcacgcccgagcggctgggcacggtgttcgcggccaaggtggatgccgctcttttgctggatgagctgacgcggggtatggagctgtcggcgttcgtgctgttctcctcggccgcggggatcctggggtcggccgggcagggcaactacgccgcggccaatgccgctctggacgcgctggcgtaccggcggcgggcggcgggtctgccgggggtgtcgctggcgtgggggctgtgggaagaggccagcgggatgaccgggcatctggccggcaccgaccaccggcgcatcatccgttccggtctgcatcccatgtcgaccccggacgcactggccctcttcgatgcggccctggctctggaccggccggtcctgctgcccgccgacctgcgtcccgccccgcccctgccgcccctgctgcaggacctcctgcccgccacccgccgccgcaccacccgcaccaccactaccggtggtgcggacaacggcgcccagctgcacggccggctggccggccagacacacgaacaacagcacaccaccctcctcgccctggtccgctcccacatcgccaccgtcctgggccacaccacccccgacaccatcccccccgaccgcgcgttccgcgacctcggcttcgactccctcaccgccgtcgaactacgcaaccggctctcccacaccaccggactccgcctccccaccaccctcgccttcgaccaccccaaccccaccaccctcacccaccacctccacacacaactcgtcagcaagggactcaccgccgcggccgagccggacgccgcaacgacacccccggggctgccctcgctgctctcggagctcgagcggctggaggcggtagtgctctcctccaccacatcctccgctgccccgctggacgacggcgcgcgcacgcggctggcctcccgactgcattccctcgcccagaagttgaacggcgacgacaccgcccccgacctcgcagagacatcggacgaggagatgttcgctctcatcgacagggaagtcggattcgaatctcaatga

SEQ ID NO: 3 - DNA, 11916 bp, Artificial Sequence
  Description of Artificial Sequence In vitro mutagenized DNA
gtgcagaggatggacggcggggaagaaccccgccctgcggcaggggaggtcctcggagtggccgacgaggcggacggcggcgtcgtcttcgtttttcccgggcagggcccgcaatggccgggcatgggaagggaacttctcgacgcttccgacgtcttccgggagagcgtccgcgcctgcgaagccgcgttcgcgccctacgtcgactggtcggtggagcaggtgttgcgggactcgccggacgctcccgggctggaccgggtggacgtcgtccagccgaccctgttcgccgtcatgatctccctggccgccctctggcgctcgcaaggggtcgagccgtgcgcggtgctgggacacagcctgggcgagatcgcggcagcccacgtctcgggaggcctgtccctggccgacgccgcacgcgtggtgacgctttggagccaggcacagaccacccttgccgggaccggcgcgctcgtctccgtcgccgccacgccggatgagctcctgccccgaatcgctccgtggaccgaggacaacccggcgcggctcgccgtcgcagccgtcaacggaccccggagcacagtcgtttccggtgcccgcgaggccgtcgcggacctggtggccgacctcaccgccgcgcaggtgcgcacgcgcatgatcccggtggacgttcccgcccactcccccctgatgtacgccatcgaggaacgggtcgtcagcggcctgctgcccatcaccccacgcccctcccgcatccccttccactcctcggtgaccggcggccgcctcgacacccgcgagctagacgcggcgtactggtaccgcaacatgtcgagcacggtccggttcgagcccgccgcccggctgcttctgcagcaggggcccaagacgttcgtcgagatgagcccgcacccggtgctgaccatgggcctccaggagctcgccccggacctgggcgacaccaccggcaccgccgacaccgtgatcatgggcacgctgcgccgcggccagggcaccctggaccacttcctgacgtctctcgcccaactacgggggcatggtgagacgtcggcgaccaccgtcctctcggcacgcctgaccgcgctgtcccccacgcagcagcagtcgctgctcctggacctggtgcgcgcccacaccatggcggtgctgaacgacgacggaaacgagcgcaccgcgtcggatgccggcccatcggcgagtttcgcccacctcggcttcgactccgtcatgggtgtcgaactgcgcaaccgcctcagcaaggccacgggcctgcggttgcccgtgacgctcatcttcgaccacaccacgccggccgcggtcgccgcgcgccttcggaccgcggcgctcggccacctcgacgaggacaccgcgcccgtaccggactcacccagcggccacggaggcacggcagcggcggacgacccgatcgccatcatcggcatggcatgccgtttcccgggcggagtccggtccccgaaggacctgtgggagctggccgcctcgggcggagacgccatcgggccgttccccaccgaccgcggatggcccacggaacagcgtcacgcccaggaccccacgcagcccggcacgttctatccgcagggaggcgggttccttcacgacgcggcgcacttcgacgccggcttcttcggaatcagtccacgtgaggcactggcgatggatccgcagcagcggctgctgctggagacgtcctgggaggcgttcgagcgggcgggaatcgatccgctgtcggtacgcgggtcccgtacgggcgtcttcgcgggcgccctctccttcgactacggcccgcgtatggacaccgcgtcgtcggagggcgccgcggacgtggagggccacatcctcaccggtaccacgggcagcgtcctgtcgggccgtatcgcctacagcttcgggctggaagggccggcgatcaccgtggacacggggggctcggcatcgctcgtgacgctgcatctggcgtgccagtcgctgcggtcgggtgagtgcacgctcgcgctggccggcggcgtctcggtcatgtccaccctcggcatgttcatcgagttctcccggcagcgcgggctgtcggtggacggcaggtgcaaggcgtactcggctgcagccgacggcaccggctggggcgagggcgtcgggatgctgttggtggagcggttgtcggatgcggtgcggctggggcatcgggtgctggcggtggtacgcggcagtgcggtcaaccaggacggtgcgtcgaatgggctgacggcgccgaacggtccggctcaggagcgggtgatccggcaggcgttggcgaacgcggggttgtccgtggcggatgtggatgtggtggaggggcacgggacgggcacgacgctgggtgatccgatcgaggcacaggcgttgctcgccacgtacgggcagcgggccggtgacaggccgctgtggctggggtctctgaagtccaacatcgggcacaccatggctgccgcgggtgtgggtggggtcatcaagatggtgatggcgttgcgggagggggtgttgccgcggacgttgcatgtggataagccgtcgccgcaggtggactggtccgcgggggcggtgcggctgctgacggaggcggtgccgtggccgggggacgcggcagggcggttgcggcgggcgggagtgtcgtcgttcgggatcggcggcacgaatgcgcatgtgattttggaggaggcgccggcggcggggggctgtgttgccgggggtggggtgttggagggtgctccgggtcttgccatttcggtggctgagtcggtggccgctccagtggctgtgtctgcgccggtggctgagtcggtgccggtgccggtgccggtgccggttcctgtgccggtgtcggctaggtctgaggctgggttgcgggcgcaggcggaggcgttgcgtcagtacgtggcagtccggccggacgtttcgcttgccgatgtgggtgcgggtctggcctgtgggcgggctgtgctggagcatcgtgcggtcgtcctggccgcggaccgtgaggagctggtgcaagggttgggggcgctggcggcgggtgagccggatcggcgggtgaccacgggtcatgcgccgggtggtgaccggggcggtgtcgtcttcgtgtttcccggacagggtgggcagtgggccgggatgggtgtgcgtctgctcgcctcctctccggtgttcgcccggcggatgcaggcgtgcgaggaggctctggcgccgtgggtggactggtctgtggtggacatcctgcgccgggacgcgggggatgcggtgtgggagcgggccgatgtggtccagcctgtgctgttcagcgtcatggtgtctttggctgctctgtggcgttcctacggtatcgaacccgacgcggtccttggccattcccagggcgagatcgcggccgcgcatgtgtgtggggcgctgagcctgaaggacgcggcgaagactgttgcgctgcgcagccgggcgctggccgctgtgcggggccggggcggcatggcctcagtgccgctgcctgcccaggaggtggagcagctcattggtgagcggtgggcggggcggttgtgggtggcggcggtcaacggcccccgctccaccgccgtctcgggggatgccgaggcggtggacgaggtgctggcgtactgtgccggcaccggggtgcgggcccggcggatcccggtcgactatgcctcgcactgcccccatgtgcagcccctgcgggaggagttgctggagctgctgggggacatcagcccgcagccgtccggcgtgccgttcttctccacggtggagggcacctggctggacaccacaaccctggacgccgcctactggtaccgcaacctgcaccagccggtccgtttcagcgatgccgtccaggccctggcggatgacggacaccgcgtcttcgtcgaagtcagcccccaccccaccctcgtccccgccatcgaagacaccaccgaagacaccgccgaagacgtcaccgcgatcggcagcctccgccgcggcgacaacgacacccgccgcttcctcaccgccctcgcccacacccataccaccggcatcggcacacccaccacctggcaccaccactacacccaccaccacacccacccccacccccacacgcacctcgacctgcccacctaccccttccaacaccagcactactggctcgagagctcacagccgggtgccggatccggttcgggtgccggtgccggttcgggtgccggttccgggcgggcagggactgcgggcgggacggcagaggtggagtcgcggttctgggacgcggtggcccgccaggacctggaaacggtcgcgaccacactcgccgtgcccccctccgccggcctggacacggtggtgcccgcactctccgcctggcaccgccaccaacacgaccaagcccgcatcaacacctggacctaccaggaaacctggaaacccctcaccctccccaccacccaccaaccccaccaaacctggctcatcgccatccccgaaacccagacccaccacccccacatcaccaacatcctcaccaacctccaccaccacggcatcacccccatccccctcaccctcaaccacacccacaccaacccccaacacctccaccacaccctccaccacacccgacaacaagcccaaaaccacaccaccggagccatcaccggcctgctctccctcctcgccctcgacgaaacaccccacccccaccacccccacacacccaccggcaccctcctcaacctcaccctcacccaaacccacacccaaacccacccaccaacccccctctggtacgccaccaccaacgccaccaccacccaccccaacgaccccctcacacaccccacccaagcccaaacctggggactcgcccgcaccaccctcctcgaacaccccacccacaccgccggaatcatcgacctccccaccacccccaccccccacaccctccagcacctcacccaaaccctcacccaaccccaccaccaaacccaactcgccatccgcaccaccggcacccacacccgccgcctcacccccaccaccctcacccccacacaccaaccacccacccccaccccccacggaaccaccctcatcaccggcggaaccggcgccctcgccacccacctcacccaccacctcaccacccaccaacccacccaacacctcctcctcaccagccgaaccggcccccacaccccccacgcacaacacctcaccacccaactccaacaaaaaggcatccacctcaccatcaccacctgcgacaccagcaacccagaccaactccaacaactcctcaacaccatccccccacaacaccccctcaccaccgtcatccacaccgcaggcatcctcgacgacgccaccctcaccaacctcacccccacccaactcaacaacgtcctccgcgccaaagcccacagcgcccacctcctccaccaactcacccaacacacccccctcaccgccttcgtcctctactcctccgccgccgccaccttcggcgcacccggccaagccaactacgccgcagccaacgcctacctcgacgccctcgcccaccaccgccacacccaccacctccccgccaccagcatcgcctggggcacctggcaaggaaacggactcgctgattcggacaaggcccgcgcatatctcgaccgccgcgggtttcgacccatgtcacccgagttggccacggcagcggtcacgcaggcgatcgcggacaccgaacggccgtatgtcgtcatcgccgacatcgactggagcaagatcgaacacacctctcagaccagcgacctggtgagcgcggcccgggaaagggagccagctgtccagcgccccactccaccggcggagttgcacaaaacgctggcccatcagacgtcggccgaccaacgggccgcattgctcgagctcgtacgagaccatgtggcggcagtgctccggcacgcggacccgaaagccatcgcgcccgaccagtcgttccgtgcactcggcttcgattcactcacggccgtcgagttccgaaacctgctgatcaaggcaacaggactccgccttcctgtctcgctggtcttcgaccacccgacccctgccaaactcgccgtacacctgcagaaccaactgcggggcacagcagcggagtcggctccttcagcggcagccgttaccgccgaggcttctgtcaccgagccgatcgccatcgttggcatggcctgtcgtttccccggcggagtgacctcggcggacgacttctgggatctgatctcctccgagcaggacgcgatcggcggattccccaccgaccgcggctgggacctggacacgctctacgaccccgaccccgaccaccccggcacctgctacacccgaaacggcggattcctctacgacgcaggccacttcgacgccgaattcttcggcatcagcccccgcgaagccctcgccatggacccccagcaacgactcctcctcgaaaccgcctgggaaaccatcgaacacgccggcatcaacccccacaccctccacggcacccccaccggagtcttcaccggcaccaacggacaggactacgcacttcgcgtgcacaacgcgggccagtcaaccgatggtttcgcactgaccggaaccgccggcagcgtcatctccggtcgtatctcgtacacgtttggttttgagggtcctgcggtgtcggtggacacggcttgttcctcgtcgttggtggctttgcatctggcctgtcaggcgttgcgtgcgggtgagtgctcgatggcgcttgccgggggtgtgacggtgatgtcgtctccgggtgccttcgtggagttttcgcggcagcggggtctggccgcggacgggcattgcaaggcgttctcggcggcggcggacgggaccggctggggtgagggtgtggggatgctgctggtggagcggctctccgacgcccatcgcaacggtcaccgtgtcctggccgtggtgcgtggcagtgcggtcaaccaggacggtgcgagcaacggtctgaccgcgcccaacgggccgtcccagcagcgtgtcatccgccaggccctcgccaacgccggcttgtcggccggtgatgtcgacgcggtggaggcccacggcaccggcaccactttgggcgacccgatcgaggcccaggccctcctcgcgacctacggacaggaccgtgccggcgaggggccgctgtggctgggctcggtcaagtccaatgtcggtcacacacaggctgccgcgggcgtcgccggggtgatcaagatggtgatggcgctgcggcatggtctgctgccgcggacgttgcatgtggatgagccgtcgccgcatgtggactggtccgcgggtgcggtgcagctgctgacggagacggtgccctggcccggcggggaggggcggctacggcgggcaggagtgtcatcattcggcgtcagcggcaccaacgcccacgtcatcctcgaagaagcacccgccgacgacgttccggggggaccacccgccggcgagggtgacgcgggcagcgacgatgaggctgctgccggcagtcctggggtgtggccgtggctggtgtcggccaagtcgcagccggccctgcgcgcccaggcccaggccctgcacgcccacctcaccgaccaccccggcctcgacctcgcggatgtcggatacaccctcgcccacgcccgcgccgtgttcgaccaccgcgccaccctcatcgccgcggaccgcgacacgttcctgcaagcactccaggcactcgccgcaggcgagccccaccccgccgtcatccacagcagcgccccgggcgggaccgggaccggggaggccgcaggaaagaccgcattcatctgctccggacagggcacccaacgccccggcatggcccacggcctctaccacacccaccccgtcttcgccgccgcactcaacgacatctgcacccacctcgacccccacctcgaccaccccctcctccccctcctcacccaaaacgacaacgacaacgaggacgcggccgcactgctccagcagacccgctacgcccagcccgccctcttcgccttccaggtcgccctccaccgcctcctcaccgacggctaccacatcaccccccactactacgccggacactccctcggcgaaatcaccgccgcccacctcgccggcatcctcaccctcaccgacgccaccaccctcatcacccaacgcgccaccctcatgcaaaccatgccccccggcaccatgaccaccctccacaccaccccccaccacatcacccaccacctcaccgcccacgaaaacgacctcgccatcgccgccatcaacacccccacctccctcgtcatcagcggcaccccccacaccgtccaacacatcaccaccctctgccaacaacaaggcatcaaaaccaaaaccctccccaccaaccacgccttccactccccccacaccaaccccatcctcaaccaactccaccagcacacccaaaccctcacctaccacccaccccacacccccctcatcaccgccaacaccccacccgaccaactcctcaccccccactactggacccaacaagcccgcaacaccgtcgactacgccaccaccacccaaaccctccaccaacacggcgtcaccacctacatcgaactcggacccgacaacaccctcaccaccctcacccaccacaacctccccaacccccccaccaccaccctcaccctcacccacccccaccaccacccccaaacccacctcctcaccaacctcgccaaaaccaccaccacctggcacccccaccactacacccaccacgacaaccaaccccacacccacacccacctcgacctccccacctaccccttccaacaccaccactactggctcgaaagcacacagcccggtgccggcaacgtgtcagcagccggactcgaccccaccgaacaccccctactcggcgccacattggaactggcgactgacggtggagcgcttcttgcagggcgcttgtctttgaggtcgcatccgtggctggctgaccatgccgtcggcggcacggtgctgctgtcgggcgccaccttcctcgaactcgcccttcatgcgggcacatacgtgggctgcgaccgagtggatgagctgacgctgcatgcgccgctggtggttcctgtggatgggggtgtgagtgtgcaggttggggttgcggctgcggatggggaggggcggcgtttggtgagtgtgtatgcgcggggtgggagtgcttgtggtgggggtggtgcgtcgggtggggtgtggacgtgtcatgcctcgggggtgctggttgaggctgctgctggtggtgtggtggtggatggtctggcgggggtgtggccgccgcggggtgcggtggcggtggatgtcgatggtgtccgtgaccgtttggctggggctggttgtgttttggggccggtgttttcggggctgcgtgcggtgtggcgtgatgggggggatttgctggctgaggtgtgtctgccggaggaggcgtggggtgatgcggctggttttgggctgcatccggcgttgctggatggtgtggtccagccgttgtcggtgttgcttccgggtgggacggggtttggggagggggcggggttcggggagggtgttcgggtgccggctgtgtggggtggtgtgtcgcttcaccgggcgggtgtgaccggtgtgcgggtgcgtgtgtcggctgtcgggcggggcggcgggcgtgaggcggtgtcggtcgtggtcggggatgaggcgggtgtgccggtggcgtcggtcgatcgtcttgagttgcggcctgtggatatgggtcagttgcgtgctgtctcggtttcggcggggcggcggggttcgctgtatgcggtgcagtgggctgaggtgggtcctgtgccggtgtgtgggcaggcgtgggcgtggcacgaggacgtgggtgagagcggtggtgggcctgtgccgggggtggtggtgttgcggtgcccggatgccggtgccggtggcggtggcggtggcggtggtggcggtggtgtgggtgaggttgttggtggggtgttgggtgtggtgcaggggtggctggggctggagcggtttgcgggttcgcggctggtggtggtgacccggggtgcggtggtggccggcccggaggacggcccggtggatgtggtgggtgcgtcggtgtgggggctggtgcgttcggcgcaggctgagcatccggaccggtttgtcctcctcgacctcgacaccgacaccggcaccgacctcgacaccggtgctggtgctggttggggcgtggatggtgggcgtgtggcggcggtggtggcgtgtggtgagccgcagttggcggtgcgtggggagcggttgctggccgcacgcctgaaacgacttgagtcatccggtgatgttccagcccagcggtccggtgacacacgagcccggcggtccgacgtgcctgcccagcgctccggtggcgtgcctgctcggcggtcggttgatgtatcgggtcgggaggtgttgccgtggttgtcgggtgggtcggtgttggtgacgggtgggacgggtgtgctgggtgcggcggtggcgcggcatctggctggtgtgtgtggggtgcgggatctgctgttggtgagccggcgtggtccggatgctccgggtgcggagggtctgcgggcggagctggccgcgttgggggcggaggtgcggattgttgcgtgtgatgtgggggagcggcgggaggtggtccggctgctggagggtgttcctgccgggtgtccgctgacgggtgtcgtgcatgcggctggtgtgctggacgatgcgacgatcgcctctctcacgcccgagcggctgggcacggtgttcgcggccaaggtggatgccgctcttttgctggatgagctgacgcggggtatggagctgtcggcgttcgtgctgttctcctcggccgcggggatcctggggtcggccgggcagggcaactacgccgcggccaatgccgctctggacgcgctggcgtaccggcggcgggcggcgggtctgccgggggtgtcgctggcgtgggggctgtgggaagaggccagcgggatgaccgggcacctggccggcaccgaccaccggcgcatcatccgttccggtctgcatcccatgtcgaccccggacgcactggccctcttcgatgcggccctggctctggaccggccggtcctgctgcccgccgacctgcgtcccgccccgcccctgccgcccctgctgcaggacctcctgcccgccacccgccgccgcaccacccgcaccaccactaccggtggtgcggacaacggcgcccagctgcacgcccggctggccggccagacacacgaacaacagcacaccaccctcctcgccctggtccgctcccacatcgccaccgtcctgggccacaccacccccgacaccatcccccccgaccgcgcgttccgcgacctcggcttcgactccctcaccgccgtcgaactacgcaaccggctctcccgcaccaccggactccgcctccccaccaccctcgccttcgaccaccccaaccccaccaccctcacccaccacctccacacacaactccagccacaaccggacaacgctgtcgcccccgtgttggcggagctcgacaaactcgaatccgccctctccgccctcgacaaaaccgacagcgccagcgaaagagtcaccctgcggctgaagtcactcatgttgaggtggaacgcaccccagcatccgacagccgaaagcgctgatgacgacgagaagttcacatcggcaacagaggctgagattttcaaattcattgacaacgacctcggcctgtcc

SEQ ID NO: 4 - protein, 3972 aa, Streptomyces avermitilis
VQRMDGGEEPRPAAGEVLGVADEADGGVVFVFPGQGPQWPGMGRELLDASDVFRESVRACEAAFAPYVDWSVEQVLRDSPDAPGLDRVDVVQPTLFAVMISLAALWRSQGVEPCAVLGHSLGEIAAAHVSGGLSLADAARVVTLWSQAQTTLAGTGALVSVAATPDELLPRIAPWTEDNPARLAVAAVNGPRSTVVSGAREAVADLVADLTAAQVRTRMIPVDVPAHSPLMYAIEERVVSGLLPITPRPSRIPFHSSVTGGRLDTRELDAAYWYRNMSSTVRFEPAARLLLQQGPKTFVEMSPHPVLTMGLQELAPDLGDTTGTADTVIMGTLRRGQGTLDHFLTSLAQLRGHGETSATTVLSARLTALSPTQQQSLLLDLVRAHTMAVLNDDGNERTASDAGPSASFAHLGFDSVMGVELRNRLSKATGLRLPVTLIFDHTTPAAVAARLRTAALGHLDEDTAPVPDSPSGHGGTAAADDPIAIIGMACRFPGGVRSPKDLWELAASGGDAIGPFPTDRGWPTEQRHAQDPTQPGTFYPQGGGFLHDAAHFDAGFFGISPREALAMDPQQRLLLETSWEAFERAGIDPLSVRGSRTGVFAGALSFDYGPRMDTASSEGAADVEGHILTGTTGSVLSGRIAYSFGLEGPAITVDTGCSASLVTLHLACQSLRSGECTLALAGGVSVMSTLGMFIEFSRQRGLSVDGRCKAYSAAADGTGWGEGVGMLLVERLSDAVRLGHRVLAVVRGSAVNQDGASNGLTAPNGPAQERVIRQALANAGLSVADVDVVEGHGTGTTLGDPIEAQALLATYGQRAGDRPLWLGSLKSNIGHTMAAAGVGGVIKMVMALREGVLPRTLHVDKPSPQVDWSAGAVRLLTEAVPWPGDAAGRLRRAGVSSFGIGGTNAHVILEEAPAAGGCVAGGGVLEGAPGLAISVAESVAAPVAVSAPVAESVPVPVPVPVPVPVSARSEAGLRAQAEALRQYVAVRPDVSLADVGAGLACGRAVLEHRAVVLAADREELVQGLGALAAGEPDRRVTTGHAPGGDRGGVVFVFPGQGGQWAGMGVRLLASSPVFARRMQACEEALAPWVDWSVVDILRRDAGDAVWERADVVQPVLFSVMVSLAALWRSYGIEPDAVLGHSQGEIAAAHVCGALSLKDAAKTVALRSRALAAVRGRGGMASVPLPAQEVEQLIGERWAGRLWVAAVNGPRSTAVSGDAEAVDEVLAYCAGTGVRARRIPVDYASHCPHVQPLREELLELLGDISPQPSGVPFFSTVEGTWLDTTTLDAAYWYRNLHQPVRFSDAVQALADDGHRVFVEVSPHPTLVPAIEDTTEDTAEDVTAIGSLRRGDNDTRRFLTALAHTHTTGIGTPTTWHHHYTHHHTHPHPHTHLDLPTYPFQHQHYWLESSQPGAGSGSGAGAGSGAGSGRAGTAGGTAEVESRFWDAVARQDLETVATTLAVPPSAGLDTVVPALSAWHRHQHDQARINTWTYQETWKPLTLPTTHQPHQTWLIAIPETQTHHPHITNILTNLHHHGITPIPLTLNHTHTNPQHLHHTLHHTRQQAQNHTTGAITGLLSLLALDETPHPHHPHTPTGTLLNLTLTQTHTQTHPPTPLWYATTNATTTHPNDPLTHPTQAQTWGLARTTLLEHPTHTAGIIDLPTTPTPHTLQHLTQTLTQPHHQTQLAIRTTGTHTRRLTPTTLTPTHQPPTPTPHGTTLITGGTGALATHLTHHLTTHQPTQHLLLTSRTGPHTPHAQHLTTQLQQKGIHLTITTCDTSNPDQLQQLLNTIPPQHPLTTVIHTAGILDDATLTNLTPTQLNNVLRAKAHSAHLLHQLTQHTPLTAFVLYSSAAATFGAPGQANYAAANAYLDALAHHRHTHHLPATSIAWGTWQGNGLADSDKARAYLDRRGFRPMSPELATAAVTQAIADTERPYVVIADIDWSKIEHTSQTSDLVSAAREREPAVQRPTPPAELHKTLAHQTSADQRAALLELVRDHVAAVLRHADPKAIAPDQSFRALGFDSLTAVEFRNLLIKATGLRLPVSLVFDHPTPAKLAVHLQNQLRGTAAESAPSAAAVTAEASVTEPIAIVGMACRFPGGVTSADDFWDLISSEQDAIGGFPTDRGWDLDTLYDPDPDHPGTCYTRNGGFLYDAGHFDAEFFGISPREALAMDPQQRLLLETAWETIEHAGINPHTLHGTPTGVFTGTNGQDYALRVHNAGQSTDGFALTGTAGSVISGRISYTFGFEGPAVSVDTACSSSLVALHLACQALRAGECSMALAGGVTVMSSPGAFVEFSRQRGLAADGHCKAFSAAADGTGWGEGVGMLLVERLSDAHRNGHRVLAVVRGSAVNQDGASNGLTAPNGPSQQRVIRQALANAGLSAGDVDAVEAHGTGTTLGDPIEAQALLATYGQDRAGEGPLWLGSVKSNVGHTQAAAGVAGVIKMVMALRHGLLPRTLHVDEPSPHVDWSAGAVQLLTETVPWPGGEGRLRRAGVSSFGVSGTNAHVILEEAPADDVPGGPPAGEGDAGSDDEAAAGSPGVWPWLVSAKSQPALRAQAQALHAHLTDHPGLDLADVGYTLAHARAVFDHRATLIAADRDTFLQALQALAAGEPHPAVIHSSAPGGTGTGEAAGKTAFICSGQGTQRPGMAHGLYHTHPVFAAALNDICTHLDPHLDHPLLPLLTQNDNDNEDAAALLQQTRYAQPALFAFQVALHRLLTDGYHITPHYYAGHSLGEITAAHLAGILTLTDATTLITQRATLMQTMPPGTMTTLHTTPHHITHHLTAHENDLAIAAINTPTSLVISGTPHTVQHITTLCQQQGIKTKTLPTNHAFHSPHTNPILNQLHQHTQTLTYHPPHTPLITANTPPDQLLTPHYWTQQARNTVDYATTTQTLHQHGVTTYIELGPDNTLTTLTHHNLPNPPTTTLTLTHPHHHPQTHLLTNLAKTTTTWHPHHYTHHDNQPHTHTHLDLPTYPFQHHHYWLESTQPGAGNVSAAGLDPTEHPLLGATLELATDGGALLAGRLSLRSHPWLADHAVGGTVLLSGATFLELALHAGTYVGCDRVDELTLHAPLVVPVDGGVSVQVGVAAADGEGRRLVSVYARGGSACGGGGASGGVWTCHASGVLVEAAAGGVVVDGLAGVWPPRGAVAVDVDGVRDRLAGAGCVLGPVFSGLRAVWRDGGDLLAEVCLPEEAWGDAAGFGLHPALLDGVVQPLSVLLPGGTGFGEGAGFGEGVRVPAVWGGVSLHRAGVTGVRVRVSAVGRGGGREAVSVVVGDEAGVPVASVDRLELRPVDMGQLRAVSVSAGRRGSLYAVQWAEVGPVPVCGQAWAWHEDVGESGGGPVPGVVVLRCPDAGAGGGGGGGGGGGVGEVVGGVLGVVQGWLGLERFAGSRLVVVTRGAVVAGPEDGPVDVVGASVWGLVRSAQAEHPDRFVLLDLDTDTGTDLDTGAGAGWGVDGGRVAAVVACGEPQLAVRGERLLAARLKRLESSGDVPAQRSGDTRARRSDVPAQRSGGVPARRSVDVSGREVLPWLSGGSVLVTGGTGVLGAAVARHLAGVCGVRDLLLVSRRGPDAPGAEGLRAELAALGAEVRIVACDVGERREVVRLLEGVPAGCPLTGVVHAAGVLDDATIASLTPERLGTVFAAKVDAALLLDELTRGMELSAFVLFSSAAGILGSAGQGNYAAANAALDALAYRRRAAGLPGVSLAWGLWEEASGMTGHLAGTDHRRIIRSGLHPMSTPDALALFDAALALDRPVLLPADLRPAPPLPPLLQDLLPATRRRTTRTTTTGGADNGAQLHARLAGQTHEQQHTTLLALVRSHIATVLGHTTPDTIPPDRAFRDLGFDSLTAVELRNRLSRTTGLRLPTTLAFDHPNPTTLTHHLHTQLQPQPDNAVAPVLAELDKLESALSALDKTDSASERVTLRLKSLMLRWNAPQHPTAESADDDEKFTSATEAEIFKFIDNDLGLS

SEQ ID NO: 5 - protein, 6239 aa, Streptomyces avermitilis
MQLANEAKLLEYLKRVTADLDRTRRRLYEVVEREQEPIAIVGMACRYPGGATSPTRLWHLVKSQTDAIGEFPTDRGWNLEQLYDPDPDRSGTSYTRSGGFLYDAGDFDAAFFELSPREALAMDPQQRLLLETTWETFEQGGIDPRSMRGSRTGVFVGINPEDYTTGYTHQPSNAVEGYLLTGSAASIASGRISYNFGLEGPAITIDTACSSSLVALHLACQALRSGECTMALAGGASVMATPFVFTEFSRQRGLAADGRCKAFSAAADGTGWSEGVGMLLVERLSDARRNGHRVLAVVRGSAVNQDGASNGLTAPNGRSQVKVIRQALANAHLSPADVDAVEAHGTGTTLGDPIEAQALVEAYGQDRPNGRPLWLGTLKSNIGHSMAAAGVGGVIKMVMALRNGLLPRTLHVDEPSPHVDWSAGAVQLLTETVPWPGGEGRLRRAGVSSFGVSGTNAHVILEEAPAHNIPSDTPADDVPGESAADEDAGSGDEAAAGSPGVWPWLVSAKSQPALRAQAQALHAHLTDHPGLDLADVGYTLAHARAVFDHRATLIAADRDTFLQALQALAAGEPHPAVIHSSAPGGTGTGEAAGKTAFICSGQGTQRPGMAHGLYHTHPVFAAALNDICTHLDPHLDHPLLPLLTQDPNTQDTTTLEEAAALLQQTRYAQPALFAFQVALHRLLTDGYHITPHYYAGHSLGEITAAHLAGILTLTDATTLITQRATLMQTMPPGTMTTLHTTPHHITHHLTAHENDLAIAAINTPTSLVISGTPHTVQHITTLCQQQGIKTKTLPTNHAFHSPHTNPILNQLHQHTQTLTYHPPHTPLITANTPPDQLLTPHYWTQQARNTVDYATTTQTLHQHGVTTYIELGPDNTLTTLTHDNLPNTPTTTLTLTHPHHHPQTHLLTNLAKTTTTWHPHHYTHHHNQPHTHTHLDLPTYPFQHHHYWLQPPGKPSDPSPSEGREQATTPSTPLRDVLVGKSPQERDEELLRLVRTHAAAVLGHATPEVIVPNKAFKELGFDSLAAIQLRNRLLADVDLPLPATLIFDYPTPMALCQFLRAAIVGADTGTTTRLPLTAVPADEPIAIVGMACRYPGDVRTVDDLWQVVSGGHDAIGGFPTNRGWDLDTLYNPDPDHHGTSYTRSGGFLYDAGNFDPDFFGISPREALAMDPQQRLLLETAWESIEHACINPDSLRGTPTGVFAGLTYHDYAARFPTAPAGFEGYLGHGSAGSIASGRVAYALGLEGPALTVDTACSSSLVALHLACQALRSGECSMALAGGVTVMSTPAGFVEFSRQRGLAVDGRCKAFSAAADGTGWGEGVGMLLVERLSDARRLGHRILAVVRGSAVNQDGASNGLTAPNGPSQERVIRLALANADLTPADVDAVEAHGTGTTLGDPIEAQALLATYGQDRPGNEPLWLGSMKSNIGHAQAAAGVGGVIKMVMALRNGLLPRTLHVDEPSPHVDWSAGAVQLLTETVPWPGGEGRLRRAGVSSFGVSGTNAHVILEEAPAHNIPSDTPADDAPGEAAADDVPGEAAGDDAGTGGEATGPAAGSPGVWPWLVSAKSQPALRAQAQALHAHLTDHPGLDLADVGYTLAHARAVFDHRATLIAADRDTFLQALQALAAGEPHPAVIHSSAPGGTGTGEAAGKTAFICSGQGTQRPGMAHGLYHTHPVFAAALNDICTHLDPHLDHPLLPLLTQDPNTQDTTTLEEAAALLQQTPYAQPALFAFQVALHRLLTDGYHITPHYYAGHSLGEITAAHLAGILTLTDATTLITQRATLMQTMPPGTMTTLHTTPHHITHHLTAHENDLAIAAINTPTSLVISGTPHTVQHITTLCQQQGIKTKTLPTKNAFHSPHTNPILNQLHQHTQTLTYHPPHTPLITANTPPDQLLTPHYWTQQARNTVDYATTTQTLHQHGVTTYIELGPDNTLTTLTHHNLPNTPTTTLTLTHPHHHPQTHLLTNLAKTTTTWHPHHYTHHHNQPHTHTHLDLPTYPFQHQHYWLESTQPGAGSGSGSGSGRAGTAGGTAEVESRFWDAVARQDLETVATTLAVPPSAGLDTVVPALSAWHRHQHDQARINTWTYQETWKPLTLPTTHQPHQTWLIAIPETQTHHPHITNILTNLHHHGITPIPLTLNHTHTNPQHLHHTRQQAQNHTTGPITGLLSLLALDETPHPHHPHTPTGTLLNLTLTQTHTQTHPPTPLWYATTNATTTHPNDPLTHPTQAQTWGLARTTLLEHPTHTAGIIDLPTTPTPHTLHHLTQTLTQPHHQTQLAIRTTGTHTRRLTPTTLTPTHQPPTPTPHGTTLITGGTGALATHLTHHLTTHQPTQHLLLTSRTGPHTPHAQHLTTQLQQKGIHLTITTCDTSNPDQLQQLLNTIPPQHPLTTVIHTAGILDDATLTNLTPTQLNNVLRAKAHSAHLLHQLTQHTPLNAFVLYSSAAATFGAPGQANYAAANAYLDALAHHRHTHHLPATSIAWGTWQGNGLATGQVSEHLRRRGMFAMPPELAVTAVDGAIASGRPSLLVADIDWKKLGPVLSSKSSVLLEDLPQAQGTEEARSTVEQTESTNLRQLLMGRSRSEQEEELLSLVRIHSAAVLGRDDSEAIPPGRLFRDLGFDSLAAVELRNHLAAQTELALPTTLVFDYPSPTKLAQFLLSEIAEFQPDNSTPLPRPRAELDEPIAIVGMACRFPGGVTSADDFWDLISSEQDAIGGFPTDRGWDLDTLYDPDPDHPGTCYTRNGGFLYDAGHFDAEFFGISPREALAMDPQQRLLLETAWETIEHAGINPHTLHGTPTGVFTGTNGQDHAAHIRQAPSGTEGFVLTGAATSIASGRISYILGLEGPAVTLDTACSSSLVALHLACQSLRSGECTMALAGGATVMTTPITFTEFARQRGLAPDGRCKAFSAAADGTGWGEGVGMLLVERLSDARRNGHRVLAVVRGSAVNQDGASNGLTAPNGPSQQRVIRQALANADLTPADVDAVEAHGTGTTLGDPIEAQAILATYGQDRPGNGPLWLGSVKSNVGHTQAAAGVAGVIKMVMALRHRTLPPTLHADEPSPHVDWSAGAVQLLTETVPWPGGEGRPRRAGVSSFGVSGTNAHVILEEAPADDVPGGPPADEDAGSGEEAAAGSPGVWPWLVSAKSQPALRAQAQALHAHLTDHPGLDLADVGYTLAHARAVFDHRATLIAADRDTFLQALQALAAGEPHPAVIHSSAPGGTGTGEAAGKTAFICSGQGTQRPGMAHGLYHTHPVFAAALNDICTHLDPHLDHPLLPLLTQNDNDNDNEDAAALLQQTPYAQPALFAFQVALHRLLTDGYHITPHYYAGHSLGEITAAHLAGILTLTDATTLITQRATLMQTMPPGTMTTLHTTPHHITHHLTAHENDLAIAAINTPTSLVISGTPHTVQHITTLCQQQGIKTKTLPTNHAFHSPHTNPILNQLHQHTQTLTYHPPHTPLITANTPPDQLLTPHYWTQQARNTVDYATTTQTLHQHGVTTYIELGPDNTLTTLTHHNLPNTPTTTLTLTHPHHHPQTHLLTNLAKTTTTWHPHHYTHHHNQPHTHTHLDLPTYPFQHHHYWLELPSAQTSPGQRRSRRSAPDTAESEFWDAVNEEDLQSLAETLDIDASALDTVVPALSAWHRHQHDQARINTWTYQETWKPLTLPTTHQPHQTWLIAIPETQTHHPHITNILTNLHHHGITPIPLTVNHTHTNPQHLHHTLHHTRQQAQNHTTGPITGLLSLLALDETPHPHHPHTPTGTLLNLTLPQTHTQTHPPTPLWYATTNATTTHPNDPLTHPTQAQTWGLARTTLLEHPTHTAGIIDLPTTPTPHTLHHLTQTLTQPHHQTQLAIRTTGTHTRRLTPTTLTPTHQPPTPTPHGTTLITGGTGALATHLTHHLTTHQPTQHLLLTSRTGPHTPHAQHLTTQLQQKGIHLTITTCDTSNPDQLQQLLNTIPPQHPLTTVIHTAGVNLFAPVSETDAESFSSVTAAKATGAAILHELLLDHETLEHFILFSSGAGAWGSGNQCAYSAANAYLDALATHRQTHGLPGASIAWGPWAGKGMSAGDAAHGYLEKRGILPMEPRMALAAFHRARAQRPNSNLIIADIDWERFVPAFTARRHSPLIEDIPEVRQAAQELEAAASTAKTTTAQPIATSLRERLARLTSSKQNQVLLGLIRTGICTVLGLRNPEGIEDQRAFRDLGFDSLTSAQFSKELAKETGLPLPPSLVFDYPTPQECAAHLRTQLVDLDDEEDAALSNALPQVAHRRTVEDEPIAIIGMACRFPGGVRSADDLWELLASGKDAIGVFPTDRGWDLDTLYDPDPDHPGTCYTRNGGFLYGAGHFDAEFFGISPREALAMDPQQRLLLETAWETIEHAGINPHTLHGTPTGVFAGINAQDHAAHIRQSRDVETIEGYALTGSSGSVASGRVAYTLGLEGPAVSVDTACSSSLVALHWAAQALRAGECSMALAGGVTVMSSPGTFVEFSRQRGLAADGRCKAYSAAADGTGWAEGVGMLLVERLSDARRNGHRVLAVVRGSAVNQDGASNGLTAPNGPSQQRVIRQALANAGLTPADVDAVEGHGTGTTLGDPIEAQALLAAYGQHRPHHRPLWLGSLKSNIGHAQAAAGVGGVIKMVMALRNGLLPQTLHVDEPTPQVDWSTGAVQLLTQPVPWPADPAGRPRHAGVSSFGVSGTNAHIILEEAPTPQDSDTDDEPPANAPALPHPLPLPVPVSARSEAGLRAQAQALRQYVAARPDMSPADIGAGLARGRAVLEHRAVILAADREELAQALTALAAGEPHPHITTGHTRGGDRGGVVFVFPGQGGQWAGMGLTLLTSSPVFAEHIDACEKALTPWVPWSLTDILHRDPDDPAWQQADVVQPVLFSIMVSLAALWRSYGIEPDAVLGHSQGEIAAAHICGALSLKDAAKTVALRSRALAAVRGRGAMASLPLPAQDVQQLISERWEGQLWVAALNGPHSTTVSGDTKAVDEVLAHCTDTGLRAKRIPVDYASHCPHVQPLHDELLHLLGDITPQPSTVPFFSTVEGTWLDTTTLDAAYWYRNLHQPVRFSHAIQTLTDDGHRAFIEISPHPTLVPAIEDTTENTTENITATGSLRRGDNDTHRFLTALAHTHTTGIGTPTTWHHHYTQTHPHPNPHTHLDLPTYPFQHQHYWLQPPTTTTDLTTTGLTPTHHPLLTATLTLADNNTQLLTGRLSLRTHPWLTDHTVAGMVLLPGTALLELALQAGERVDCPRVEELTLHAPLVIPHTEDVTLQVTVRAADESGHRALAIHSYSGTASSADREWTRHATGLLTHHADTDHRADTHTDACLGGSWPPPGAQPIELGDVYGRMAADSDIAYGPVFQGLHAAWRFGDDVLAEVRLPEEALRDAPAAAFGVHPALLDAALHATALTPQNGDGSTENVAQESMPDRAAHQARLPFSWSGVSLHTAGSSVLRVRLSRSPQHGNAVALTAADEDGRPVVTIESLALRPVSTEELRAAADRTPEHESLFRLDWVSVPVPANAPSPTADRPWAVIGAGLPHLPGLTEHEHVTAYDEPADLLLALDRGAPPPGVLVVGGVAHTEAREYSAEAPGERGTEACEARPDVVHVGVVHTAAVHAAAAQMLARLQAWLGDERLADSRLLVLTCGAVARASGDDATDLPGAAVWGLVRSAQSEHPDRITLLDFERGTEAEPGQLATALNCGERQLAVRPGGLFTPRLVRAPRVADAVPAVPAVAVPSAGHAAVPAAGPFLPGGTVLITGGTGVLGRLVARHLVEAHGVRHLLLAGRRGPDAEGAPELRAELGGLGATVEVVACDAADRQQLADLLTRIPDDRPLTGVVHSAGILDDGVITSLSPERLGAVLRAKADAALLLDELTRGAELSAFVMFSSASAVVGSPGQGNYAAANAVLDFLAHRRRAEGLPAVSLAWGLWEEGTGMTGHLDVDDHARISRAGMRPLPTAEALALFDAALADGEPFLMPARLDLTAVRSGAASAPVPPLLQGLLQLPRSRSAAAAPGHGAPAADEAAAWRERLARQSAGERRQALLRLVRSHVAAVLGHSGADGIDASRAFRELGFDSLTAVELRNRLTAATGLRLRATLAFDFPTPAALAEHLGERLLPDQEATGEQAGDQLSGGSEEDVRSLLTSIPIGRLRDAGLLGPLLTLADTGRGASGAAAGPEDAPPSGQDTPAPVSIDEMDIDDLMDLAHGHGTAPAREPADAEDSSSSRNRTHHTHEGETA

SEQ ID NO: 6 - protein, 4881 aa, Streptomyces avermitilis
MANEEKLRDYLKRVTADLLNVRRRLQQIESGEQEPIAIVGMACRFPGGVESAEDFWELIASGRDAVGEFPVDRGWDVEAFYDPEPGRAGSSYTRRGGFLEGAAEFDAGFFGISPREALAMDPQQRLMLEVSWEALERAGIDPATLRGSRTGVFAGLMSQDYATRLLSVPDDLAGYLGNGNAGSILSGRVAYTFGFEGPAVTVDTACSSSLVALHLACQSLRTGESSFALAGGVTVMSTPGMFVEFSRQRGLSPDGRCKAYASAADGTGMSEGVGILLLERLSEAERRGHRVLAVVRGSAVNQDGASNGLTAPNGPSQQRVIRQALACAGLSVADVDVVEGHGTGTTLGDPIEAQALLATYGQRAGDTPVWLGSVKSNIGHAQAAAGVAGVIKMVMALRAGVLPRTLHVDEPSSQVDWSSGSVRVLADEVEWPGVEGRLRRAGVSAFGVSGTNAHVILEEASGGAGGGAGRLQELGPGVVSGSGVVPWVVSARSELALRGQARRLRGVVAVGGGADGVGVSPAGVGRALVSERSVFEHRAVVVAEDRDEFLHALDALAGGRPVPGVVEGRTTSGELAVLFAGQGTQRAGMGRELYEAYPVFAQAIDEICAEADTARTDPGAPGLRDVLFAPQDSPEGRLIEDTGFAQPALFAFEVALFRLLETWGLTPDYVLGHSVGELAAAHVAGMLCLADAVALVVARGRLMQGLPSGGAMVAIEASEDEILPLPDEYASRVAHAAVNGPRSIVLSGDEDAVLDLAQQWAARGRRTRRLRTSHAFHSPHMDAMLGDFRRAAEQVTFSAPRIPVVSNVTGAPLPAETMCTPDYWVEHARSTVRFADGISWLQEQGVTTCLEIGPDGTLSALAQDSLSAPARAIPALRPDQPEARSVMTALAELFVAGTAVEWAGVFEGTAREVGDGCGVELPTYAFERERFWLDVEEGSAGGSGVSGMWGGPLWEAVECGDAGVVASLLGVDEGASLGAVVSALGEWGRVRHEREVVDGWRYREVWRPVSGGGVGGLSGAWLVVSEGEAGPVDVVAEGLERCGARVVRVEVEAGCVSREVLAGHLREAVDGEAVGGVVSLVGWGSGVVQAGVASVGLVQALGDVGVGARLWCVTGGAVSVGGRDAVWGPASGVVWGLGRVVGAEAPDRWGGLVDVPELVDERVVDGLVGVLAGVGGGGESEFAVRSSGAFVRRLVRAPLEEAVAEREWRPRGTVLVTGGTGELGAHVARWMARRGAEHLLLVSRRGESAQGVEELRADLMGLGARVSVVACDAADREALAEVLRSAVPAECPLGVVVHAAGVVDDGVLEGLSSERVTGVLRAKALAAWNLHELTRGADLSGFVVFSSAAATFGPAGQGSYAAANAYVEAIVRHRRGEGLPGLAVAWGPWAGGGMAEGAVGQMRRRGLAAMTPETALVALGQALDHDETCVTVADIDWDRFTANSLPGSRLSPLISDIPEARLARETTGLDTATASPDSFSARLKAMDTAEQERALLDLVRTYAATVLGHSTPTAVRPERAFRDLGFVSVSAVELRNRLNAVTGLLLPTTLIFDYPTPSALAGYLKEQLEEGAGGQRDIAPPVPASRVDVDEPIAIVGMACRFPGGVESAEDLWELVASGRDAVGEFPVDRGWDVEAFYDPEPGRAGSSYTRRGGFLEGAAEFDAGFFGISPREALAMDPQQRLMLEVSWEALERAGIDPATLRGSTTGVFAGMCSQDYADLVRRATEDLEGYAMTGLSSSVTSGRVAYTLGLEGPAVTVDTACSSSLVALHLACQALRSGECSLALAGGVTVMSTPGAFVEFSRQRGLSPDGRCKAYGSGADGVGWAEGVGVLLVERLSEAERRGHRVLAVVRGSAVNQDGASNGLTAPNGPSQQRVIRQALACAGLSVADVDVVEGHGTGTTLGDPIEAQALLATYGQGRSGERPVWLGSVKSNIGHAQAAAGVAGVIKMVMALRAGVLPRTLHVDEPSSQVDWSSGSVRVLADEVEWPGVEGRLRRAGVSAFGVSGTNAHVILEEASGGADGGAGRLQELGPGVVSGSGVVPWVVSARSELALRGQARRLRGVVAVGGGADGVGVSPAGVGRALVSERSVFEHRAVVVAEDRDEFLHALDALAEGAPTAGVVQGVAGPAADGKIAMLFGGQGTHWEGMAQELLGSSPVFAQQMSDCAQALEPYLDWSLLDVLRGAPDAPPLQRVDVVQPVLFAVMVSLAALWRSYGVHPDAVAGHSQGEIAAAYVAGALSLDDAARVTALRSQALAALAGQGAMASVGLPVEKLEPRLATWGDRLVIAAVNGARSAVVSGEPEAVDALVEELSHEDVPARRLMVDWASHSPQVEAIQGRLLELLAPIRARTGDVPFYSTVTGERIDGTELDADYWYRNLRQVVRFRDATQALVRAGHTVFIEACPHPAVAVGVQETLDEMGDLDSLVVGSLRRGEGGLRRFLMSVAELFVGGVAVEWSGVFGSVGRGVAGGCGVELPTYAFERERFWLDVEGAPRGSGVSGQWGGQLSEAVDTVRGGMLRDCLAGLDPAAQAETVLDLVLTHAAAVLGHGTADAVVPERAFRDLGFDSLTAVELRNRLNTATGLRFPRTLVFDHPRPVALAAHIHEQLSGGSPTTGTALALALRAPAPRVDVDEPIAIVGMACRFPGGVESAEDFWELIASGRDAVGEFPVDRGWDVEAFYDPEPGRAGTSYTRCGGFLQGAAEFDAGFFGISPREALAMDPQQRLMLEVSWEALERAGIDPATLHGSTTGVFAGVSQQDYAELLRRGTQDHEGYALTGVSNSVVSGRLSYTFGFEGPAVTVDTACSSSLVALHLACQALRSGECSLALAGGVTVMSTPGAFVEFSRQRGLSPDGRCKAYGSGADGVGWAEGVGVLLVERLSEAERRGHRVLAVVRGSAVNQDGASNGLTAPNGPSQQRVIRQALACAGLSVADVDVVEGHGTGTTLGDPIEAQALLATYGQGRSGERPVWLGSVKSNIGHAQAAAGVAGVIKMVMALNHELLPTSLHIDEPSPHIDWSSGGVRLLTEPVPWQQNGRPRRAGVSAFGVSGTNAHVIIEQAPVEAHVISEPVPAEAHVIVEQAPVEAPHVVDATGPADLTEPQEEAAEPECVADAVTEMSAEPECVADAMSEMSAECVAEAVSDKSAEPECVADAMSDKPALLPIPWLLSAKSERALRGQARRLRQFAARASDARPADVAHALAAQRSVFDHRAVVVAEDRDGFLQALDALAEGRSADGLIEGSVGPRGGHSGRRRGKTAMLFAGQGTQRVGMGRQLYAAHPAYADALDQVLAELDGHLDQPLRPLIHASADLADVADAADVLDRTRYAQPALFAVQVALFRHLERLGVRADFVAGHSIGELAAAHVAGVLPLAAACRLVAARGRLMEQLAPGGAMVAVRASEAEARQALDGREARVSVAAVNGPASVVFSGAEDEVGNMADWFAERGRRVKRLRTGHAFHSPLMDPMLEEFQQVAASLTYSEPAIPMVSTLTGDIVAAGELSDPEYWVRQVRRTVRFGDAISRLHTDGVRTFMELGPDGTLSALAEECLEATADSHPADDDTGTPQENLLIPLLRPDSPEPGTLLTGLARLHTHGAAAVNWPAALPERDRARHLDLPTYAFDHHRYWVDTSAGHPGDLSAAGLGTAGHPLLGSAVALAESQELLFTGRLSLRTHPWLADHAIFGTVLLPGTAILELAVRAGDEVDCGTVEELTLRTPLVLPEQGSVILQLSVGAPQGPQTPEEPERRTFALYAREDDGLSSSSAAATGTEWTCHATGVLTGTARPAEEHTQEPWPPADAAPVDLDGWYEQLAGAGLGYGPVFQGLREVWRRGDEVFAVVTLPESTEGQAADAARYALHPALLDAALHPVVLRHEGDAAADGHGWLPFSWTGVTVAASGASTLHVRLTVRTDEDAVGLLATDASGRIVISAGSLAFRPVSAEQLQAARTGYHDHLFRIEWRPLHLPTTPARTADWALIGPGARRTAAVLERNGASWQAYPDPAALAEALAAGAPAPGMVVISCEPDGASAPTDSALTDSALTDSAPAGSAPADSTALADATRQATTRVLALLQEWVADERLAACRLALLTHGSVTATPDEPVSDLAHAAVWGLVRSVQTENPDRFLLADTDDTDASRNALPLLAGEPQIALRNGAVRIPRMTRVPVRQPQPSTTDADWDPEATVLITGGTGVLGRLVARHLATAHGVRHLLLATRRGTAADGAADLVAELAGLGAEATVAACDIGDRAAVAALLDQVPAQHPLKAVIHTAGVVDDGILTSLTPERMEAVLHAKAFGAAHLHDLTRDAGLTTFTVFSSAAASFGSPGQGNYTAANAFLDALMQHRHTQALPGRSLAWGLWGEADGMTRNLAGTDFARMARGGLLPLSNAQGLALLDTADRLGPFGDGLLLATRLDAATLHAQATAGALPRILHGLIRIPARRSADHGIATDTPATLRERLAGLTIPAQRTGLLLELVRTHAAAVLGHPTSAVTAADGALPDDLVPADTEFRDLGFDSLTAVELRNRINAVTGLRLPATLIFDQPSPAALADHLATRLTAEAGTPDEPAPAAAAAGAGSAGSAETGQQRSTGSEKQQTRGGTSTETVESLFWIGHDTRRIEESMALLSAASFFRPAFTDPSDIPEPTFVRLAQGEARAQGEALARGETRPALICLPTVAAVSSVYQYSRFAAGLNGHRDVWYVPAPGFLEGEPLPSGIGAVTRMFADAIVRFTDGAPFALAGHSAGGWFVYAVTSHLERLGVRPEAVVTMDAYLPDDGIAPVASALTSEIFDRVTQFVDVDYTRLVAMGGYFRIFSGWSPPDITTPALFLRGRDGEQMPPPWGVPHTVLDIQGNHFTMLEQFADSTARHVDEWLTEIASVRR

SEQ ID NO: 7 - protein, 5532 aa, Streptomyces avermitilis
MDTSSEKLVDALRASLKANQTLRARNEQLAAAMEASSEPIAIVGMACRFPGGVCSPEELWELVASGGDAIGEFPAGRGWDLEGLFDSDPDRSGTSYARYGGFLYEAGEFDADFFGISPREALAMDPQQRLLLETSWEAFERAGIDPLSMRGSRTGVFAGVMYHDYGSRLGTIPEGFEGYIGNGSGGAVASGRVAYTLGLEGPAVSVDTACSSSLVALHLACQSLRSGECTLALAGGVTVMSTPHLFVEFSRQRGLSVDGRCKSFAGGADGTGMGEGVGMLLVERLSDAVRLGHRVLAVLRGSAVNQDGASNGLTAPNGPAQERVIRQALANAGLSVADVDVVEGHGTGTTLGDPIEAQALLATYGQRAGNRPLWLGSVKSNIGHAQAAAGVGGVIKMVMALREGVLPRTLHVDEPSPQVDWSAGAVRLLTEAVPWPGDAAGRLRRAGVSSFGVSGTNAHVILEEAPAAGGCVAGGGVLEGAPGLAISVAESVAAPVAVSAPVAESVPVPVPVPVPVPVSARSEAGLRAQAEALRQYVAVRPDVSLADVGAGLACGRAVLEHRAVVLAADREELVQGLGALAAGEPDRRVTTGHAPGGDRGGVVFVFPGQGGQWAGMGVRLLASSPVFARRMQACEEALAPWVDWSVVDILRRDAGDAVWERADVVQPVLFSVMVSLAALWRSYGIEPDAVLGHSQGEIAAAHVCGALSLKDAAKTVALRSRALAAVRGRGGMASVPLPAQEVEQLIGERWAGRLWVAAVNGPRSTAVSGDAEAVDEVLAYCAGTGVRARRIPVDYASHCPHVQPLREELLELLGDISPQPSGVPFFSTVEGTWLDTTTLDAAYWYRNLHQPVRFSDAVQALADDGHRVFVEVSPHPTLVPAIEDTTEDTAEDVTAIGSLRRGDNDTRRFLTALAHTHTTGIGTPTTWHHHYTHHHTHPHNHHLDLPTYPFQRQHYWLDAPTGAGDVAAAGLEPAEHPLLAATVQLADTDGCLLTGRLSLRSHPWLGDYEVGGAVLLSGSAFVELAVQVGERVGCTRIEQLTVHAPLVVPVGGGVSVQVGVAAADGEGRRLVSVYARGGSACGGGGASGGVWTCHASGVLVEAAAGGGVVVDGLAGVWPPRGAVAVDVDGVRDRLAGAGCVLGPVFSGLRAVWRDGGDLLAEVCLPEEAWGDAAGFGLHPALLDGVVQPLSVLLPGGTGFGEGAGFGEGVRVPAVWGGVSLHRAGVTGVRVRVWAVGRGGGREAVSVVVGDEAGVPVASVDRLELRPVDMGQLRAVSVSAGRRGSLYAVQWAEVGPVPVCGQAWAWHEDVGESGGGPVPGVVVLRCPDAGAGGGGGGGVGEVVGGVLGVVQGWLGLERFAGSRLVVVTRGAVVAGQEDGPVDVVGAAVWGLVRSAQAEHPDRFVLLDLDTDTDTGTDLDTGAGAGAGAGWGVDGGHVAAVVACGEPQLAVRGERVLAARLTRLESSVDVPAQRSGDVAGREVLPWLSGGSVLVTGGTGVLGAAVARHLAGVCGVRDLLLVSRRGPDAPGAEGLRAELAALGAEVRIVACDVGERREVVRLLEGVPAGCPLTGVVHAAGVLDDATIASLTPERLGTVFAAKVDAALLLDELTRGMELSAFVLFSSAAGILGSAGQGNYAAANAALDALAYRRRAAGLPGVSLAWGLWEEASGMTGHLAGTDHRRIIRSGLHPMSTPDALALFDAALALDRPVLLPADLRPAPPLPPLLQDLLPATRRRTTRTTTTGGADNGAQLHARLAGQTHEQQHTTLLALVRSHIATVLGHTTPDTIPPDRAFRDLGFDSLTAVELRNRLSRTTGLRLPTTLAFDHPNPTTLTHHLHTQLLGSDSTASIPAPRAAAVPADQDEPVAIIGMACRYPGGVTSAEELWELLASGRDTVGEFPTDRGWDLEALFDPEPGRPGTSYTRCGSFLYDAGEFDAGFFGISPREALAMDPQQRLLLEASWEAMEQAGIDPTTVRGSQTGVFAGLIPQAYGPRLHENAAADTEGYVLTGTSGSVASGRISYTFGFEGPAVSVDTACSSSLVALHLACQALRAGECSMALAGGVTVMSSPGAFVEFSRQRGLAADGHCKAFSAAADGTGWGEGVGMLLVERLSDARRNGHRVLAVVRGSAVNQDGASNGLTAPNGPSQQRVIRQALANAGLSAGDVDAVEAHGTGTTLGDPIEAQALLATYGQDRAGEGPLWLGSVKSNVGHTQAAAGVAGVIKMVMALRNGLLPRTLHVDEPSPHVDWSAGAVQLLTETVPWPGGEGRLRRAGVSSFGVSGTNAHVILEEAPAHNIPSDTPADDVPGGPPAGEDAGSGEEAAAGSPGVWPWLVSAKSQPALRAQAQALHAHLTDHPGLDLADVGYTLAHARAVFDHRATLIAADRDTFLQALQALAAGEPHPAVIHSSAPGGTGTGEAAGKTAFICSGQGTQRPGMAHGLYHTHPVFAAALNDICTHLDPHLDHPLLPLLTQDPNTQDTTTLEEAAALLQQTPYAQPALFAFQVALHRLLTDGYHITPHYYAGHSLGEITAAHLAGILTLTDATTLITQRATLMQTMPPGTMTTLHTTPHHITHHITAHENDLAIAAINTPTSLVISGTPHTVQHITTLCQQQGIKTKTLPTNHAFHSPHTNPILNQLHQHTQTLTYHPPHTPLITANTPPDQLLTPHYWTQQARNTVDIATTTQTLHQHGVTTYIELGPDNTLTTLTHHNLPNTPTTTLTLTHPHHHPQTHLLTNLAKTTTTWHPHHYTHHHNQPHTHTHLDLPTYPFQHHHYWLESTQPGAGNVSAAGLDPTEHPLLGATLELAEGDGCLLTGRLSLRTHPWLAGHAVGGVVLLPGTAFAELALHAGESVGCDHVDELTLHTPLVIPEVGDVTLQVAIAAPDESGRRMMTIHSRGEGGSGGADASASAWTRHAAGVLSPAKDDDTASYELLAGPWPPVGATPVDLNTAYDQMADAGFAYGLAFQGLRAAWRYGDDILVEARLPEEVSGDAAAYGLHPALLDAALQGTGLLSVAGPGTPVVPHVWNGLRFRTHGAVSVRACLSTLGATGAAVCVRITDDTGVPVASVDRLELRPVDMGQLRAVSVSAGRRGSLYAVQWAEVGPVPVCGQAWAWHEDVGESGGGPVPGVVVLRCPDAGADGGGGGGVGEVVGGVLGVVQGWLGLERFAGSRLVVVTRGAVVAGPEDGPVDVVGAAVWGLVRSAQAEHPDRFVLLDLDTDLDSGADADAGNEAGMGSGLDGGRVAAVVACGEPQLAVRGERVLAARLTRLESPVDVSGREVLPWLSGGSVLVTGGTGVLGAAVARHLAGVCGVRDLLLVSRRGPDAPGAEGLRAELAALGAEVRIVACDVGERREVVRLLEGVPAGCPLTGVVHAAGVLDDATIASLTPERLGTVFAAKVDAALLLDELTRGMELSAFVLFSSAAGILGSAGQGNYAAANAALDALAYRRRAAGLPGVSLAWGLWEEASGMTGHLAGTDHRRIIRSGLHPMSTPDALALFDAALALDRPVLLPADLRPAPPLPPLLQDLLPATRRRTTRTTTTGGADNGAQLHARLAGQTHEQQHTTLLALVRSHIATVLGHNAPEMIPVDSAFRDLGFDSLTAVELRNRLGEATGLRLPTSLVFDQPNAATLARHLRRELMGDDAEGETPSQVALHQVAADEPIAIVGMACRFPGGVCSPEELWELVASGGDAIGEFPAGRGWDLEGLFDSDPDRSGTSYARYGGFLYEAGEFDADFFGISPREALAMDPQQRLLLETSWEAFERAGIDPLSMRGSRTGVFAGVMYHDYAARLHHVPEGFEGLIANGSAGSVATGRVAYSFGLEGPAVTVDTACSSSLVALHWAAQALRAGECSMALAGGVTVMSSPGTFVEFSRQRGLAADGRCKAYSAAADGTGWAEGVGMLLVERLSDARRNGHRVLAVVRGSAVNQDGASNGLTAPNGPSQQRVIRQALANAGLTPADVDAVEGHGTGTTLGDPIEAQALLAAYGQHRPHHRPLWLGSLKSNIGHAQAAAGVGGVIKMVMALRNGLLPQTLHVDEPTPQVDWSTGAVQLLTQPVPWPADPAGRPRHAGVSSFGVSGTNAHVILEEAPAAAGGAAGGGVSVGAPNPALPVAESEPVPVPVPVSARSEAGLRAQAQALRQYVAARPDMSPADIGAGLARGRAVLEHRAVILAADREELAQALTALAAGEPHPHITTGHTRGSDRGGVVFVFPGQGGQWAGMGLTLLTSSPVFAEHIDACEKALTPWVPWSLTDILHRDPDDPAWQQADVVQPVLFSIMVSLAALWRSYGIEPDAVLGHSQGEIAAAHICGALSLKDAAKTVALRSQALAAVRGRGAMVSLPLPAQDVQQLISERWEGQLWVAALNGPHSTTVSGDTTAVEELLTHCADTGLRAKRIPVDYASHCPHVQPLHDELLHLLGDITPQPSTMPFFSTVVGHLVWYTTTLDAAYWYRNLHQPVRFSHAIQTLTDDGHRPFIEISPHPTLVPAIEDTTENTTENITATGSLRRGDNDTHRFLTALAHTHTTGIRTPTTWHHHYTQTHPHPHNHHLDLPTYPFQHQHYWLQPPTTTTDLTTTGLTPTHHPLLTATLTLANNNTQLLTGRLSLRTHPWLTDHTVVGTTLVPGTALLELALQATTTDHLEELALHTPLVIPREGAVDVQVHINPPDDTDTRSLTIYSRSENAPAAAPWRHHATAVLGTKTSRIETGRSHDDLSMWPPAGAVRCADEELAALYGDYEANGFVYGPAFRGLTAAWRLGDEVFAEVRLPEQVHGEASAYNLHPALLDAALHAAAFAPSGSLPQGSVPFSFTGVTLHAANASSLRVRLSPADPNSGHAAVSVLVTDDTGTPVASVEALAVRPLAADELRAAERAVQRAELFDMKWVEVPSDVLVSGGASVVVLDGADDLVGLAAEEDGVPGVVVLRCPDAGADGGGGGGGVGEVVGGVLGVVQGWLGLERFAGSRLVVVTRGAVVAGPEDGPVDGPVDVVGAAVWGLVRSAQAEHPDRFVLLDLDTDLDSGADRDAGNEAGMGSGLDGGRVAAVVACGEPQLAVRGERVLAARLTRLESPVDVSGREVLPWLSGGSVLVTGGTGVLGAAVARHLAGVCGVRDLLLVSRRGPDAPGAEGLRAELAALGAEVRIVACDVGERREVVRLLEGVPAGCPLTGVVHAAGVLDDATIASLTPERLGTVFAAKVDAALLLDELTRGMELSAFVLFSSAAGILGSAGQGNYAAANAALDALAYRRRAAGLPGVSLAWGLWEEASGMTGHLAGTDHRRIIRSGLHPMSTPDALALFDAALALDRPVLLPADLRPAPPLPPLLQDLLPATRRRTTRTTTTGGADNGAQLHGRLAGQTHEQQHTTLLALVRSHIATVLGHTTPDTIPPDRAFRDLGFDSLTAVELRNRLSHTTGLRLPTTLAFDHPNPTTLTHHLHTQLVSKGLTAAAEPDAATTPPGLPSLLSELERLEAVVLSSTTSSAAPLDDGARTRLASRLHSLAQKLNGDDTAPDLAETSDEEMFALIDREVGFESQ

SEQ ID NO: 8 - protein, 3972 aa, Artificial Sequence
  Description of Artificial Sequence Synthetic protein; one amino acid is sustituted
VQRMDGGEEPRPAAGEVLGVADEADGGVVFVFPGQGPQWPGMGRELLDASDVFRESVRACEAAFAPYVDWSVEQVLRDSPDAPGLDRVDVVQPTLFAVMISLAALWRSQGVEPCAVLGHSLGEIAAAHVSGGLSLADAARVVTLWSQAQTTLAGTGALVSVAATPDELLPRIAPWTEDNPARLAVAAVNGPRSTVVSGAREAVADLVADLTAAQVRTRMIPVDVPAHSPLMYAIEERVVSGLLPITPRPSRIPFHSSVTGGRLDTRELDAAYWYRNMSSTVRFEPAARLLLQQGPKTFVEMSPHPVLTMGLQELAPDLGDTTGTADTVIMGTLRRGQGTLDHFLTSLAQLRGHGETSATTVLSARLTALSPTQQQSLLLDLVRAHTMAVLNDDGNERTASDAGPSASFAHLGFDSVMGVELRNRLSKATGLRLPVTLIFDHTTPAAVAARLRTAALGHLDEDTAPVPDSPSGHGGTAAADDPIAIIGMACRFPGGVRSPKDLWELAASGGDAIGPFPTDRGWPTEQRHAQDPTQPGTFYPQGGGFLHDAAHFDAGFFGISPREALAMDPQQRLLLETSWEAFERAGIDPLSVRGSRTGVFAGALSFDYGPRMDTASSEGAADVEGHILTGTTGSVLSGRIAYSFGLEGPAITVDTGGSASLVTLHLACQSLRSGECTLALAGGVSVMSTLGMFIEFSRQRGLSVDGRCKAYSAAADGTGWGEGVGMLLVERLSDAVRLGHRVLAVVRGSAVNQDGASNGLTAPNGPAQERVIRQALANAGLSVADVDVVEGHGTGTTLGDPIEAQALLATYGQRAGDRPLWLGSLKSNIGHTMAAAGVGGVIKMVMALREGVLPRTLHVDKPSPQVDWSAGAVRLLTEAVPWPGDAAGRLRRAGVSSFGIGGTNAHVILEEAPAAGGCVAGGGVLEGAPGLAISVAESVAAPVAVSAPVAESVPVPVPVPVPVPVSARSEAGLRAQAEALRQYVAVRPDVSLADVGAGLACGRAVLEHRAVVLAADREELVQGLGALAAGEPDRRVTTGHAPGGDRGGVVFVFPGQGGQWAGMGVRLLASSPVFARRMQACEEALAPWVDWSVVDILRRDAGDAVWERADVVQPVLFSVMVSLAALWRSYGIEPDAVLGHSQGEIAAAHVCGALSLKDAAKTVALRSRALAAVRGRGGMASVPLPAQEVEQLIGERWAGRLWVAAVNGPRSTAVSGDAEAVDEVLAYCAGTGVRARRIPVDYASHCPHVQPLREELLELLGDISPQPSGVPFFSTVEGTWLDTTTLDAAYWYRNLHQPVRFSDAVQALADDGHRVFVEVSPHPTLVPAIEDTTEDTAEDVTAIGSLRRGDNDTRRFLTALAHTHTTGIGTPTTWHHHYTHHHTHPHPHTHLDLPTYPFQHQHYWLESSQPGAGSGSGAGAGSGAGSGRAGTAGGTAEVESRFWDAVARQDLETVATTLAVPPSAGLDTVVPALSAWHRHQHDQARINTWTYQETWKPLTLPTTHQPHQTWLIAIPETQTHHPHITNILTNLHHHGITPIPLTLNHTHTNPQHLHHTLHHTRQQAQNHTTGAITGLLSLLALDETPHPHHPHTPTGTLLNLTLTQTHTQTHPPTPLWYATTNATTTHPNDPLTHPTQAQTWGLARTTLLEHPTHTAGIIDLPTTPTPHTLQHLTQTLTQPHHQTQLAIRTTGTHTRRLTPTTLTPTHQPPTPTPHGTTLITGGTGALATHLTHHLTTHQPTQHLLLTSRTGPHTPHAQHLTTQLQQKGIHLTITTCDTSNPDQLQQLLNTIPPQHPLTTVIHTAGILDDATLTNLTPTQLNNVLRAKAHSAHLLHQLTQHTPLTAFVLYSSAAATFGAPGQANYAAANAYLDALAHHRHTHHLPATSIAWGTWQGNGLADSDKARAYLDRRGFRPMSPELATAAVTQAIADTERPYVVIADIDWSKIEHTSQTSDLVSAAREREPAVQRPTPPAELHKTLAHQTSADQRAALLELVRDHVAAVLRHADPKAIAPDQSFRALGFDSLTAVEFRNLLIKATGLRLPVSLVFDHPTPAKLAVHLQNQLRGTAAESAPSAAAVTAEASVTEPIAIVGMACRFPGGVTSADDFWDLISSEQDAIGGFPTDRGWDLDTLYDPDPDHPGTCYTRNGGFLYDAGHFDAEFFGISPREALAMDPQQRLLLETAWETIEHAGINPHTLHGTPTGVFTGTNGQDYALRVHNAGQSTDGFALTGTAGSVISGRISYTFGFEGPAVSVDTACSSSLVALHLACQALRAGECSMALAGGVTVMSSPGAFVEFSRQRGLAADGHCKAFSAAADGTGWGEGVGMLLVERLSDAHRNGHRVLAVVRGSAVNQDGASNGLTAPNGPSQQRVIRQALANAGLSAGDVDAVEAHGTGTTLGDPIEAQALLATYGQDRAGEGPLWLGSVKSNVGHTQAAAGVAGVIKMVMALRHGLLPRTLHVDEPSPHVDWSAGAVQLLTETVPWPGGEGRLRRAGVSSFGVSGTNAHVILEEAPADDVPGGPPAGEGDAGSDDEAAAGSPGVWPWLVSAKSQPALRAQAQALHAHLTDHPGLDLADVGYTLAHARAVFDHRATLIAADRDTFLQALQALAAGEPHPAVIHSSAPGGTGTGEAAGKTAFICSGQGTQRPGMAHGLYHTHPVFAAALNDICTHLDPHLDHPLLPLLTQNDNDNEDAAALLQQTRYAQPALFAFQVALHRLLTDGYHITPHYYAGHSLGEITAAHLAGILTLTDATTLITQRATLMQTMPPGTMTTLHTTPHHITHHLTAHENDLAIAAINTPTSLVISGTPHTVQHITTLCQQQGIKTKTLPTNHAFHSPHTNPILNQLHQHTQTLTYHPPHTPLITANTPPDQLLTPHYWTQQARNTVDYATTTQTLHQHGVTTYIELGPDNTLTTLTHHNLPNPPTTTLTLTHPHHHPQTHLLTNLAKTTTTWHPHHYTHHDNQPHTHTHLDLPTYPFQHHHYWLESTQPGAGNVSAAGLDPTEHPLLGATLELATDGGALLAGRLSLRSHPWLADHAVGGTVLLSGATFLELALHAGTYVGCDRVDELTLHAPLVVPVDGGVSVQVGVAAADGEGRRLVSVYARGGSACGGGGASGGVWTCHASGVLVEAAAGGVVVDGLAGVWPPRGAVAVDVDGVRDRLAGAGCVLGPVFSGLRAVWRDGGDLLAEVCLPEEAWGDAAGFGLHPALLDGVVQPLSVLLPGGTGFGEGAGFGEGVRVPAVWGGVSLHRAGVTGVRVRVSAVGRGGGREAVSVVVGDEAGVPVASVDRLELRPVDMGQLRAVSVSAGRRGSLYAVQWAEVGPVPVCGQAWAWHEDVGESGGGPVPGVVVLRCPDAGAGGGGGGGGGGGVGEVVGGVLGVVQGWLGLERFAGSRLVVVTRGAVVAGPEDGPVDVVGASVWGLVRSAQAEHPDRFVLLDLDTDTGTDLDTGAGAGWGVDGGRVAAVVACGEPQLAVRGERLLAARLKRLESSGDVPAQRSGDTRARRSDVPAQRSGGVPARRSVDVSGREVLPWLSGGSVLVTGGTGVLGAAVARHLAGVCGVRDLLLVSRRGPDAPGAEGLRAELAALGAEVRIVACDVGERREVVRLLEGVPAGCPLTGVVHAAGVLDDATIASLTPERLGTVFAAKVDAALLLDELTRGMELSAFVLFSSAAGILGSAGQGNYAAANAALDALAYRRRAAGLPGVSLAWGLWEEASGMTGHLAGTDHRRIIRSGLHPMSTPDALALFDAALALDRPVLLPADLRPAPPLPPLLQDLLPATRRRTTRTTTTGGADNGAQLHARLAGQTHEQQHTTLLALVRSHIATVLGHTTPDTIPPDRAFRDLGFDSLTAVELRNRLSRTTGLRLPTTLAFDHPNPTTLTHHLHTQLQPQPDNAVAPVLAELDKLESALSALDKTDSASERVTLRLKSLMLRWNAPQHPTAESADDDEKFTSATEAEIFKFIDNDLGLS

SEQ ID NO: 9 - DNA, 32 bp, Artificial Sequence
  Description of Artificial Sequence primer based on the sequence between 1954 and 1985 of SEQ ID NO 1
accgtggacacggggggctcggcatcgctcgt

SEQ ID NO: 10 - DNA, 28 bp, Artificial Sequence
  Description of Artificial Sequence antisense primer based on the sequence between 1758 and 1776 of SEQ ID NO 1
ataagcttaatcgatccgctgtccggta

SEQ ID NO: 11 - DNA, 30 bp, Artificial Sequence
  Description of Artificial Sequence antisense primer based on the sequence between 2710 and 2729 of SEQ ID NO 1
atgaattccctccaaaatcacatgcgcatt

(19) United States
(12) Patent Application Publication
(10) Pub. No.: US 2004/0101936 A1
(43) Pub. Date: May 27, 2004

(54) Process for producing avermectin derivative

(76) Inventors: Hirofumi Endo, Chiyoda-ku, Tokyo (JP); Hiroyuki Yamaguchi, Machida-shi, Tokyo (JP); Yutaka Kanda, Sunto-gun, Shizuoka (JP); Shinichi Hashimoto, Yamaguchi (JP); Satoshi Omura, Tokyo (JP); Haruo Ikeda, Kanagawa (JP)

Correspondence Address: Nixon & Vanderhye, 8Th Floor, 1100 North Glebe Road, Arlington, VA, 22201-4714, US

(21) Appl. No.: 10/204,862

(22) PCT Filed: Feb. 23, 2001
(86) PCT No.: PCT/JP01/01381

(30) Foreign Application Priority Data
Feb. 24, 2000 (JP) 2000-47405

(51) Int. Cl. C07H 21/04; C12P 17/02; C12N 9/10; C12N 1/21; C12N 15/74

(52) U.S. Cl. 435/123; 435/69.1; 435/193; 435/252.3; 435/320.1; 536/23.2

(57) ABSTRACT

According to the present invention, 22,23-dihydroavermectin B1a, which is useful as a medicine, a veterinary drug, and a pesticide, can be directly fermented and produced. This can obviate the need for the complicated and difficult conventional processes for purifying avermectin B1a at an industrial level and for chemically modifying avermectin B1a and can significantly decrease cost and time required in the industrial production of 22,34-dihydroavermectin B1a. The production of the formulation containing only 22,23-dihydroavermectin B1a, which is highly effective as a medicine, is also realized.

### TECHNICAL FIELD

The present invention relates to a process for producing 22,23-dihydroavermectin B1a or a derivative thereof, which is useful as a medicine, a substrate compound and a modified avermectin aglycon synthase used in the production, and a gene encoding the enzyme.

### BACKGROUND ART

A conventional process for producing 22,23-dihydroavermectin B1a involves a method comprising extracting an avermectin mixture with organic solvents from various microorganisms producing a plurality of avermectins, purifying avermectin B1 in the extract, and reducing the carbon bond between the 22nd and 23rd positions of avermectin B1 with hydrogen in the presence of a catalytic amount of compounds (Japanese Published Unexamined Patent Application No. 61198/79). A mixture of 22,23-dihydroavermectin B1a and 22,23-dihydroavermectin B1b obtained by the process, which is called 22,23-dihydroavermectin B1, is used as a medicine.

Avermectin is a polyketide compound which, as with other polyketide compounds, is biosynthesized through continuous condensation of lower fatty acids, reduction of a carbonyl group at β position of an elongated acyl group, dehydration, or enoyl reduction. These various repetitive synthetic processes of many polyketide compounds are carried out by a polymeric and multifunctional enzyme complexes, each of which has a specific active site (domain) required for each catalytic activity. A general reaction formula of polyketide biosynthesis is outlined, for example in Ann. Rev. Gen., 24, 37 (1990) and Ann. Rev. Microbiol., 47, 875 (1993).

DNA encoding a polyketide synthase usually encodes all the required activity sites for the synthesis of a polyketide backbone (aglycon), and contains modules, that is, repeating units involving condensation steps and modification steps following condensation. Depending on the genetic information existing in each module, the elongation or modification of an acyl group is determined. A polyketide synthase specifically acts on a specific carboxylic acid constitutional unit that is involved in each condensation step or acts on a site that defines the specific modifying function after condensation.

Regarding the biosynthetic mechanism of avermectin aglycon, it has been reported that, as with other polyketide compounds, avermectin aglycon contains lower fatty acids, such as acetic acid and propionic acid as its components [J. Antibiot., 39, 541-549 (1986)], and a polyketide synthase constituted by modules is present in avermectin-producing bacteria [Gene, 115, 119-125 (1992), Ann. New York Acad. of Sci., 721, 123-132 (1994)]. DNA fragments involved in the biosynthesis of avermectin (Japanese Published Unexamined Patent Application No. 15391/91) or domain structures of some modules [Ann. New York Acad. Sci., 721, 123-132 (1994)] have been reported although the nucleotide sequence, which is the basis thereof, is not disclosed. That is, the existence of some modules in the avermectin aglycon synthase is merely presumed while the structure of the entire synthase has not been elucidated. The present inventors made an intensive investigation into avermectin aglycon biosynthase genes, thereby precisely deducing the domain structure of each module involved in the biosynthesis of avermectin aglycon.

Among 22,23-dihydroavermectin B1 components, 22,23-dihydroavermectin B1a is known as a highly effective medicine [Antimicrobial Agent and Chemotherapy, 15, 372-378 (1979) and Japanese Published Examined Publication No. 54113/87]. Avermectin B1a, which is a raw material for synthesizing 22,23-dihydroavermectin B1a, is obtained by culturing avermectin B1a producing microorganisms and purifying it from the culture. Streptomyces avermitilis, which produces avermectin, produces 8 components of avermectins having analogous structures (Japanese published Examined Publication No. 17558/90). Among strains selectively producing avermectin component which were mutated and bred from Streptomyces avermitilis, any strains which produce only avermectin B1a are not obtained. Accordingly, avermectin B1a should be isolated from avermectins having analogous structures for the purpose of producing 22,23-dihydroavermectin B1a. However, since there are extraordinary similarities between avermectin structures, it is very difficult to industrially isolate only avermectin B1a. For this reason, it is considered that a currently used 22,23-dihydroavermectin preparation consists of dihydroavermectin B1a and dihydroavermectin B1b. The necessity of hydrogenation with a special catalyst after purification complicates the process for producing 22,23-dihydroavermectin B1 and results in increased cost.

Accordingly, if only 22,23-dihydroavermectin B1a can be directly produced, all the problems involved in conventional industrial production can be solved and medicines containing only 22,23-dihydroavermectin B1a, which has the highest antiparasitic activity in its component, can be produced. A process for selectively and directly producing 22,23-dihydroavermectin B1a, however, is not known yet.

### DISCLOSURE OF THE INVENTION

The object of the present invention is to provide a process for selectively and directly producing only 22,23-dihydroavermectin B1a.

The present inventors have made an intensive investigation into studies in order to attain the above object and, have found that 22,23-dihydroavermectin B1a or a derivative thereof can be directly produced by modifying a gene encoding an avermectin aglycon synthase to obtain a modified enzyme and allowing a compound, which is a substrate of the modified enzyme, to act on a cell in which the modified genes have been expressed. The present invention has been completed on the basis of this result.

The present invention relates to the following (1) to (25).

(1) A modified avermectin aglycon synthase comprising at least one domain with an eliminated or lowered activity, wherein the domain is selected from the group consisting of acyl carrier protein (ACP), β-ketoacyl ACP synthase (KS), acyltransferase (AT), β-ketoacyl ACP reductase (KR), dehydratase (DH), enoyl reductase (ER) and thioesterase (TE), which are involved in the synthesizing reaction of avermectin aglycon.

(2) The modified avermectin aglycon synthase according to (1) wherein the modified avermectin aglycon synthase is derived from Streptomyces avermitilis.

(3) The modified avermectin aglycon synthase according to (1) above, wherein the domain with an eliminated or lowered activity is selected from the group consisting of ATs, ACPs, KS1, AT1, KR1, ACP1, KS2, DH2 and KR2.

(4) A modified avermectin aglycon synthase comprising an amino acid sequence wherein one or more amino acid residues are deleted, substituted or added in the amino acid sequence of the avermectin aglycon synthase consisting of the amino acid sequences shown in SEQ ID NOs: 4, 5, 6 and 7, and having an activity for producing 22,23-dihydroavermectin B1a or a derivative thereof when the modified avermectin aglycon synthase is contacted with an N-acetylcysteamine thioester compound.

(5) The modified avermectin aglycon synthase according to (4) above, which contains a polypeptide consisting of the amino acid sequence shown in SEQ ID NO: 8.

(6) The modified avermectin aglycon synthase according to (4) above, wherein the N-acetylcysteamine thioester compound is represented by formula (I):

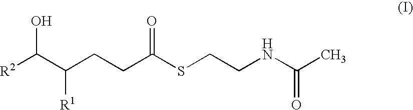

wherein R¹ and R², which may be the same or different, represent hydrogen, substituted or unsubstituted alkyl, substituted or unsubstituted alkenyl, substituted or unsubstituted aryl or substituted or unsubstituted heterocycle, or, R¹ and R², combined together, form substituted or unsubstituted cycloalkyl.

(7) The modified avermectin aglycon synthase according to (6) above, wherein the N-acetylcysteamine thioester compound is represented by formula (I) in which R¹ is methyl and R² is sec-butyl.

(8) A DNA which encodes the modified avermectin aglycon synthase according to any one of (1) to (7) above.

(9) A DNA which comprises a DNA encoding a polypeptide consisting of the amino acid sequence shown in SEQ ID NO: 8.

(10) A DNA which comprises a DNA consisting of the nucleotide sequence shown in SEQ ID NO: 3.

(11) A DNA which hybridizes with the DNA according to any one of (8) to (10) above under stringent conditions and encodes a polypeptide having an activity for producing 22,23-dihydroavermectin B1a or a derivative thereof when the modified avermectin aglycon synthase is contacted with the N-acetylcysteamine thioester compound.

(12) A recombinant DNA which is obtained by ligating the DNA according to any one of (8) to (11) above with a vector.

(13) A transformant which is obtained by introducing the recombinant DNA according to (12) above into a host cell.

(14) The transformant according to (13) above, wherein the host cell is a microorganism.

(15) The transformant according to (14) above, wherein the microorganism belongs to the genus Streptomyces.

(16) The transformant according to (15) above, wherein the microorganism belonging to the genus Streptomyces is Streptomyces avermitilis.

(17) The transformant according to (16) above, which is Streptomyces avermitilis KS1mut.

(18) An N-acetylcysteamine thioester compound, which is a substrate compound for the modified avermectin aglycon synthase according to any one of (1) to (7) above and converted to 22,23-dihydroavermectin B1a or a derivative thereof when the compound is contacted with the modified avermectin aglycon synthase.

(19) An N-acetylcysteamine thioester compound, which is represented by formula (I):

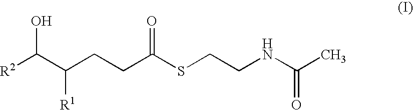

wherein R¹ and R², which may be the same or different, represent hydrogen, substituted or unsubstituted alkyl, substituted or unsubstituted alkenyl, substituted or unsubstituted aryl or substituted or unsubstituted heterocycle, or, R¹ and R², combined together, form substituted or unsubstituted cycloalkyl.

(20) The N-acetylcysteamine thioester compound according to (19) above, which is represented by formula (I), wherein R¹ is methyl and R² is sec-butyl.

(21) A process for producing an N-acetylcysteamine thioester compound which is characterized by employing as a starting material, a compound represented by formula (II):

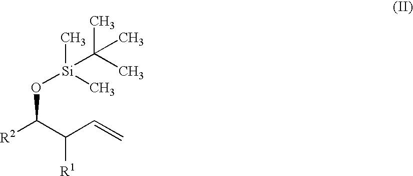

wherein R¹ and R², which may be the same or different, represent hydrogen, substituted or unsubstituted alkyl, substituted or unsubstituted alkenyl, substituted or unsubstituted aryl or substituted or unsubstituted heterocycle, or, R¹ and R², combined together, form substituted or unsubstituted cycloalkyl as a starting material, and including a reaction step of adding N-acetylcysteamine.

(22) The process for producing an N-acetylcysteamine thioester compound according to (21) above, which is characterized by employing as a starting material, a compound represented by formula (II):

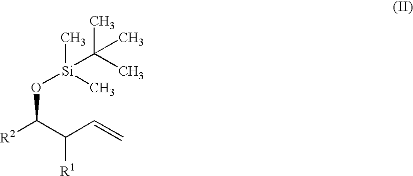

wherein R¹ and R², which may be the same or different, represent hydrogen, substituted or unsubstituted alkyl, substituted or unsubstituted alkenyl, substituted or unsubstituted aryl or substituted or unsubstituted heterocycle, or, R¹ and R², combined together, form substituted or unsubstituted cycloalkyl, and comprising the steps of:

(a) ozone-oxidating the compound, and thereafter adding carbon chains by the Wittig reaction;

(b) deprotecting t-butyldimethylsilyl group of the compound obtained in step (a) and reintroducing another protecting group using chlorotriethylsilane;

(c) reducing α-β unsaturated carbon bond of the resultant compound in the presence of a palladium-carbon catalyst, hydrolyzing an ester with potassium hydroxide, neutralizing the reaction mixture, and adding N-acetylcysteamine in the presence of a condensing agent to obtain a thioester compound; and

(d) removing the protecting group by adding acetic acid to the thioester compound.

(23) A process for producing a modified avermectin aglycon synthase, comprising the steps of:

culturing the transformant according to any one of (13) to (17) above in a medium untill a modified polypeptide having an activity of a avermectin aglycon synthase is produced and accumulated in the culture; and

collecting the polypeptide from the culture.

(24) A process for producing 22,23-dihydroavermectin B1a or a derivative thereof, comprising the steps of:

contacting a culture of the transformant according to any one of (13) to (17) above or a treated product thereof or the synthase according to any one of (1) to (7) above with the N-acetylcysteamine thioester compound according to claim 18 in a medium; and

collecting 22,23-dihydroavermectin B1a or a derivative thereof produced and accumulated in the medium.

(25) A process for producing 22,23-dihydroavermectin B1a or a derivative thereof, characterized in that an N-acetylcysteamine thioester compound is employed as a substrate compound for the modified avermectin aglycon synthase according to any one of (1) to (7) above.

“The modified avermectin aglycon synthase comprising an amino acid sequence wherein one or more amino acid residues are deleted, substituted or added in the amino acid sequence of the avermectin aglycon synthase consisting of the amino acid sequence shown in SEQ ID NOs: 4, 5, 6 and 7, and having an activity for producing 22,23-dihydroavermectin B1a or a derivative thereof when the modified avermectin aglycon synthase is contacted with an N-acetylcysteamine thioester compound” according to (4) above can be obtained by introducing site-specific mutation into DNA encoding a polypeptide having an amino acid sequence shown in SEQ ID NO: 4, 5, 6 or 7 by a site-specific mutation introducing method described in, for example, Molecular Cloning, A laboratory Manual, Second Edition, Cold Spring Harbor Laboratory Press (1989) (hereinafter abbreviated to “Molecular Cloning, 2nd Edition”), Current Protocols in Molecular Biology, John Wiley & Sons (1987-1997) (hereinafter abbreviated to “Current Protocols in Molecular Biology”), Nucleic Acids Research, 10, 6487 (1982), Proc. Natl. Acad. Sci. USA, 79, 6409 (1982), Gene, 34, 315 (1985), Nucleic Acids Research, 13, 4431 (1985), or Proc. Natl. Acad. Sci. USA, 82, 488 (1985).

The number of amino acids to be deleted, substituted or added is not particularly limited and is preferably one to several decades amino acids and particularly preferably one to several amino acids.

In order for the polypeptide of the present invention to have an activity for producing 22,23-dihydroavermectin B1a or a derivative thereof when the modified avermectin aglycon synthase is contacted with an N-acetylcysteamine thioester compound, the polypeptide is preferably at least 60%, generally at least 80%, and particularly preferably at least 95% homologous with the amino acid sequence shown in SEQ ID NO: 1 when calculated using BLAST [J. Mol. Biol., 215, 403 (1990)], FASTA [Methods in Enzymology, 183, 63(1990)] and the like.

“DNA which hybridizes under stringent conditions” according to (11) above refers to DNA that is obtained by employing DNA having a nucleotide sequence shown in SEQ ID NO: 3 as a probe through colony hybridization, plaque hybridization, Southern hybridization or the like. Specifically, it can include DNA which can be identified by performing hybridization in the presence of 0.7 to 1.0 mol/l NaCl at 65° C. using a filter having a colony- or plaque-derived DNA immobilized thereon, followed by washing the filter at 65° C. using a 0.1× to 2×SSC (saline-sodium citrate) solution [1×SSC solution (150 mmol/l NaCl, 15 mmol/l sodium citrate) wherein “n x” indicates a n-fold concentrated solution.

Hybridization can be carried out in accordance with methods described in protocols such as Molecular Cloning, 2nd Edition, Current Protocols in Molecular Biology, DNA Cloning 1: Core Techniques, and A Practical Approach, Second Edition, Oxford University (1995). Specific examples of hybridizable DNA include DNA which is at least 80% homologous, preferably at least 95% homologous with a nucleotide sequence shown in SEQ ID NO: 3 when calculated using BLAST, FASTA and the like.

The present invention will be described in detail below.

[1] Structural Analysis of Avermectin Aglycon Synthase

(1) Isolation of Avermectin Aglycon Synthase Gene and Determination of Nucleotide Sequence

Methods for isolating avermectin aglycon synthase genes include a method described in Japanese Published Unexamined Patent Application No. 15391/91 and colony hybridization described in Molecular Cloning, 2nd Edition.

More specifically, chromosomal DNA of Streptomyces avermitilis is partially digested with a suitable restriction enzyme, for example, Sau3AI. Examples include the following method. A cosmid vector, which can replicate in Escherichia coli, is cleaved at a unique restriction enzyme site, such as the BamHI site. The cleaved cosmid vector is linked to the digested chromosomal DNA, and Escherichia coli is then transformed with this recombinant DNA, and a transformant carrying avermectin aglycon synthase genes is selected from the obtained transformants by colony hybridization.

Specific examples of DNA obtained by the method can include DNA having the nucleotide sequence shown in SEQ ID NO: 1 or 2. The open reading frames (ORF) contained in these sequences are ORF1 (nucleotide nos. 1 to 11916 of SEQ ID NO: 1), ORF2 (nucleotide nos. 11971 to 30688 of SEQ ID NO: 1), ORF3 (nucleotide nos. 1 to 14643 of SEQ ID NO: 2), and ORF4 (nucleotide nos. 14824 to 31419 of SEQ ID NO: 2). Examples of the amino acid sequence of the polypeptide encoded by these sequences include sequences respectively shown in SEQ ID NOs: 4, 5, 6 and 7. FIG. 1 shows a restriction map of avermectin aglycon synthase gene regions (aveAI and aveAII) in genome DNA of Streptomyces avermitilis together with the deduced transcription unit (arrow).

(2) Deduction of Module and Domain of Avermectin Aglycon Synthase

Modules, domains and ORFs, which are relevant to the avermectin aglycon synthase genes, can be determined by comparing similarity with the sequences of 3 types of polyketide synthase domains of erythromycin [Nature, 348, 176-178 (1990), Science, 252, 675-679 (1991), Eur. J. Biochem., 204, 39-49 (1992)].

The condensation reaction, which is a basic reaction in the synthesis of polyketide, requires various catalytic activities including an acyl carrier protein (ACP), a β-ketoacyl ACP synthase (KS) and an acyltransferase (AT).

In many cases, β-carbonyl groups generated by the condensation reaction are modified. However, depending on a module, some β-carbonyl groups may not be modified and may be used for the next condensation reaction.

Catalytic activities associated with the modification of a β-carbonyl group after the condensation reaction include a β-ketoacyl ACP reductase (KR), a dehydratase (DH) and an enoyl reductase (ER). The biosynthesis of a polyketide chain is terminated by separating from a polyketide synthase by the thioesterase (TE) activity. All or several of these modification activities act in each condensation process, thereby determining the structure of a final product.

The avermectin aglycon synthase genes (aveAI and aveAII) of Streptomyces avermitilis are characterized by genes that have several open reading frames each of which comprises one or more repeating units called a module, just as the other known polyketide biosynthetic genes have. The module is defined as a gene fragment which encodes activities for a one-time synthesis, that is, a one-time condensation reaction and other various subsequent modification reactions of the β-carbonyl group. Each module encodes all or several of ACP, KS and AT associated with the condensation reaction in polyketide synthesis, and KR, DH and ER associated with the modification reaction of the β-carbonyl group. Furthermore, there is also a module which does not have any domain for a modification reaction. A polypeptide encoded by such a module is referred to as a synthase unit (SU).

FIG. 2(b) and (c) show a biosynthetic pathway of 6,8a-seco-6,8a-deoxy-5-oxo-avermectin aglycon synthesized with avermectin aglycon synthases of Streptomyces avermitilis.

PKS-1 is obviously associated with initiation reaction, since the initiation module (SUs), differing from other modules, has acyltransferase (AT) activity on the N-terminal side. PKS-3 is also obviously associated with the final reaction of polyketide, since module 9 (SU9) has a thioesterase (TE) domain.

Examples of deduced modules of avermectin synthase genes, a synthesis unit encoded by the modules, the domain constituting each synthesis unit and a subdomain which is a DNA encoding the domain, include the following sequences.

The terms used in the present invention are defined as follows.

Module represents a gene fragment encoding the activities of the one-time condensation reaction and various subsequent modification reaction of the β-carbonyl group.

Synthase unit (SU) represents a polypeptide encoded by a module.

Domain represents polypeptide having each catalytic activity constituting a synthase unit.

Subdomain represents a gene fragment encoding a domain.

These modules are represented as the following nucleotide numbers in SEQ ID NOs: 1 and 2. That is to say, the modules are shown in SEQ ID NO: 1 as,

Initiation Module: 85 to 1353,

Module 1: 1441 to 6180,

Module 2: 6256 to 11658,

Module 3: 12076 to 15147,

Module 4: 15217 to 19938,

Module 5: 20008 to 24690,

Module 6: 24781 to 30309, and,

are represented in SEQ ID NO: 2 as,

Module 7: 100 to 4692,

Module 8: 4771 to 7818,

Module 9: 7906 to 14619,

Module 10: 14935 to 20334,

Module 11: 20413 to 25734,

Module 12: 25810 to 31125.

The amino acid sequences of various synthase units (SU) encoded by these modules are represented as the following amino acids. That is to say, the sequences are represented in SEQ ID NO: 4 as,

Initiation SU: 29 to 451,

SU1: 481 to 2060,

SU2: 2086 to 3886;

in SEQ ID NO: 5 as,

SU3: 36 to 1059,

SU4: 1083 to 2656,

SU5: 2680 to 4240,

SU6: 4271 to 6113;

in SEQ ID NO: 6 as,

SU7: 34 to 1564,

SU8: 1591 to 2606,

SU9: 2636 to 4873; and,

in SEQ ID NO: 7 as,

SU10: 38 to 1837,

SU11: 1864 to 3637,

SU12: 3663 to 5434.

DNAs encoding avermectin aglycon synthase domains (subdomains) are represented as the following nucleotide numbers. That is to say, the DNAs are represented in SEQ ID NO: 1 as,

in Initiation Module,

ATs: 85 to 1032,

ACPs: 1096 to 1353;

in Module 1,

KS1: 1441 to 2742,

AT1: 3148 to 4068,

KR1: 5143 to 5676,

ACP1: 5935 to 6180;

in Module 2,

KS2: 6256 to 7545,

AT2: 7906 to 8829,

DH2: 8947 to 9384,

KR2: 10609 to 11142,

ACP2: 11413 to 11658;

in Module 3,

KS3: 12076 to 13368,

AT3: 13756 to 14694,

ACP3: 14902 to 15147;

in Module 4,

KS4: 15217 to 16506,

AT4: 16917 to 17862,

KR4: 18886 to 19419,

ACP4: 19693 to 19938;

in Module 5,

KS5: 20008 to 21297,

AT5: 21658 to 22584,

KR5: 23602 to 24138,

ACP5: 24445 to 24690;

in Module 6,

KS6: 24781 to 26079,

AT6: 26413 to 27336,

DH6: 27475 to 27894,

KR6: 29227 to 29760,

ACP6: 30064 to 30309; and,

are also represented in SEQ ID NO: 2 as,

in Module 7,

KS7: 100 to 1383,

AT7: 1648 to 2673,

KR7: 3634 to 4188,

ACP7: 4447 to 4692;

in Module 8,

KS8: 4771 to 6060,

AT8: 6322 to 7344,

ACP8: 7573 to 7818;

in Module 9,

KS9: 7906 to 9258,

AT9: 9676 to 10773,

DH9: 10885 to 11289,

KR9: 12547 to 13104,

ACP9: 13378 to 13659,

TE9: 13879 to 14619;

in Module 10,

KS10: 14935 to 16224,

AT10: 16543 to 17565,

DH10: 17689 to 18066,

KR10: 19285 to 19842,

ACP10: 20089 to 20334;

in Module 11,

KS11: 20413 to 21705,

AT11: 21991 to 23019,

DH11: 23149 to 23529,

KR11: 24685 to 25242,

ACP11: 25489 to 25734;

in Module 12,

KS12: 25810 to 27102,

AT12: 27367 to 28392,

DH12: 28516 to 28878,

KR12: 30076 to 30633,

ACP12: 30880 to 31125.

The deduced amino acid sequences of various domains encoded by these subdomains are represented as:

in SEQ ID NO: 4,

ATs: 29 to 344,

ACPs: 366 to 451,

KS1: 481 to 914,

AT1: 1050 to 1356,

KR1: 1715 to 1892,

ACP1: 1979 to 2060,

KS2: 2086 to 2515,

AT2: 2636 to 2943,

DH2: 2983 to 3128,

KR2: 3537 to 3714,

ACP2: 3805 to 3886;

in SEQ ID NO: 5,

KS3: 36 to 466,

AT3: 596 to 908,

ACP3: 978 to 1059,

KS4: 1083 to 1512,

AT4: 1653 to 1964,

KR4: 2306 to 2483,

ACP4: 2575 to 2656,

KS5: 2680 to 3109,

AT5: 32030 to 3538,

KR5: 3878 to 4056,

ACP5: 4159 to 4240,

KS6: 4271 to 4703,

AT6: 4741 to 5048,

DH6: 5095 to 5234,

KR6: 5679 to 5856,

ACP6: 5955 to 6036;

in SEQ ID NO: 6,

KS7: 34 to 461,

AT7: 550 to 891,

KR7: 1212 to 1396,

ACP7: 1483 to 1564,

KS8: 1591 to 2020,

AT8: 2108 to 2448,

ACP8: 2525 to 2606,

KS9: 2636 to 3086,

AT9: 3226 to 3591,

DH9: 3629 to 3763,

KR9: 4183 to 4363,

ACP9: 4460 to 4553,

TE9: 4627 to 4873; and,

in SEQ ID NO: 7,

KS10: 38 to 467,

AT10: 574 to 914,

DH10: 956 to 1081,

KR10: 1488 to 1673,

ACP10: 1756 to 1837,

KS11: 1864 to 2294,

AT11: 2390 to 2732,

DH11: 2776 to 2902,

KR11: 3288 to 3473,

ACP11: 3556 to 3637,

KS12: 3663 to 4093,

AT12: 4182 to 4523,

DH12: 4565 to 4685,

KR12: 5085 to 5270,

ACP12: 5353 to 5434.

[2] Preparation of Modified Avermectin Aglycon Synthase

(1) Introduction of Site-Specific Mutation

DNA which encodes a modified avermectin aglycon synthase having a mutation so as to eliminate or significantly lower the activity in at least one domain is prepared based on the above information. The domain in which the activity is eliminated or significantly lowered may be any of the above-described domains and are preferably ATs, ACPs, KS1, AT1, KR1, ACP1, KS2, DH2 and KR2.

Mutations for eliminating or significantly lowering the activity in these domains are not particularly limited. Examples thereof include the deletion or substitution of an amino acid residue in the active center. It is important that an avermectin aglycon synthase protein is produced by being translated from two large transcription units. Thus, when a termination codon or a frameshift mutation is introduced into the gene existing in the upstream domain of the transcription unit, the transcription is terminated in mid course and, in some cases, the activity of the downstream domain is not expressed. In such a case, even thought there is no mutation existing in the downstream domain of the gene, per se, the entire mutated transcription unit is considered as having been deactivated. In order to minimize the influence on the entire transcription unit, the mutation to be introduced is preferably carried out by preventing the introduction of frameshift or termination codon. More preferably, mutation is carried out by substituting a specific amino acid in an active center with another amino acid. Examples of such mutation include mutation in which serine as an active center of AT, serine as an active center of ACP, or cysteine as an active center of KS [Eur. J. Biochem., 204, 39-49 (1992)] is substituted with another amino acid. More specific examples include a mutation in which “T” represented as the nucleotide 1969 in the nucleotide sequence shown in SEQ ID NO: 1 encoding KS1 is substituted with “G.” As a result of this mutation, a cysteine residue, which is represented as the amino acid 657 in the amino acid sequence shown in SEQ ID NO: 4, is replaced with a glycine residue. The cysteine residue, which is represented as the amino acid 657 in the amino acid sequence shown in SEQ ID NO: 4 is also conserved in other ketosynthase [Eur. J. Biochem., 204, 39-49 (1992)] and is concluded to be essential in expressing the activity in this domain.

Methods for introducing mutation are not particularly limited and include: a method in which cells having DNA encoding avermectin aglycon synthase without mutation are subjected to mutation by NTG treatment or UV irradiation; a method in which DNA per se encoding avermectin aglycon synthase without mutation is processed with a mutagen such as hydroxyurea; and a method in which a site-specific mutation is introduced based on the nucleotide sequence information of the avermectin aglycon synthase gene. Among these, the method for introducing site-specific mutation based on the nucleotide sequence information is suitable because a specific mutation can be introduced into enormous genes such as avermectin aglycon synthase gene without causing any unintended mutation. For example, mutation can be introduced in accordance with methods described in Molecular Cloning, 2nd Edition, Current Protocols in Molecular Biology, Nucleic Acids Research, 10, 6487 (1982), Proc. Natl. Acad. Sci. USA, 79, 6409 (1982), Gene, 34, 315 (1985), Nucleic Acids Research, 13, 4431 (1985), and Proc. Natl. Acad. Sci. USA, 82, 488 (1985).

(2) Preparation of Cells Transformed with Recombinant DNA and Preparation of Modified Avermectin Aglycon Synthase

Methods for obtaining a modified avermectin synthase include a method using strains having the modified avermectin aglycon synthase described in (1) and a method using a transformant, which is prepared by ligating the mutagen-treated DNA or the site-specific mutation-introduced DNA described in (1) and vector DNA to prepare a recombinant DNA, and the recombinant DNA is introduced into a host cell, thereby preparing a transformant. The host cell used in the latter method includes bacteria, yeast, filamentous fungus, animal cells, plant cells, and insect cells as long as the introduced modified genes are expressible in the cell. As an expression vector, it is possible to use any vector that can autonomously replicate in the above host cells or can be integrated into chromosomes thereof and that contains a promoter at a site which permits transcription of the introduced modified genes (hereinafter referred to as DNA encoding the polypeptide of the present invention).

When a prokaryote (e.g., bacteria) is used as a host cell, a preferred recombinant vector comprising DNA which encodes the polypeptide of the present invention may be autonomously replicative in prokaryotes and comprises a promoter, a ribosome-binding sequence, the DNA of the present invention and a terminator. The vector may further comprise a gene that regulates the promoter.

Examples of expression vectors include pBTrp2, pBTac1, pBTac2 (each of which is commercially available from Boehringer Mannheim), pKK233-2 (manufactured by Pharmacia), pSE280 (manufactured by Invitrogen), pGEMEX-1 (manufactured by Promega), pQE-8, pQE-9, pQE-60, pQE-70 (each of which is manufactured by QIAGEN), pKYP10 (Japanese Published Unexamined Patent Application No. 110600/83), pKYP200 [Agric. Biol. Chem., 48, 669 (1984)], pLSA1 [Agric. Biol. Chem., 53, 277 (1989)], pGEL1 [Proc. Natl. Acad. Sci. USA, 82, 4306 (1985)], pBluescript II SK(−) (manufactured by Stratagene), pTrS30 [prepared from Escherichia coli JM109/pTrS30 (FERM BP-5407)], pTrS32 [prepared from Escherichia coli JM109/pTrS32 (FERM BP-5408)], pGHA2 [prepared from Escherichia coli IGHA2 (FERM BP-400), Japanese Published Unexamined Patent Application No. 221091/85], pGKA2 [prepared from Escherichia coli IGKA2 (FERM BP-6798), Japanese Published Unexamined Patent Application No. 221091/85], pTerm2 (U.S. Pat. No. 4,686,191, U.S. Pat. No. 4,939,094, U.S. Pat. No. 5,160,735), pSupex, pUB110, pTP5, pC194, pEG400 [J. Bacteriol., 172, 2392 (1990)], pGEX (manufactured by Pharmacia), pUC19 [Gene, 33, 103 (1985)], pUC118 (manufactured by Pharmacia), pET system (manufactured by Novagen), pIJ702, and pIJ922, etc.

Examples of chromosomal integration vectors include a vector derived from actinophage R4 [J. Bacteriol., 173, 4237 (1991)].

Examples of homologous recombination vectors include pKC7 (Japanese Published Unexamined Patent Application No. 189774/94).

Any promoter capable of functioning in host cells may be used, including promoters derived from Escherichia coli or a phage such as trp promoter (Ptrp), lac promoter (Plac), P_{L} promoter, P_{R} promoter and T7 promoter. An artificially designed, modified promoter may also be used, including a promoter obtained by binding two Ptrp promoters in tandem (Ptrp×2), tac promoter, lac T7 promoter and let I promoter.

It is preferable to use a plasmid having an appropriate distance (e.g., 6-18 nucleotides) between Shine-Dalgarno sequence (i.e., ribosome-binding sequence) and an initiation codon. In the recombinant vector of the present invention, a terminator is not necessarily required for the expression of the DNA of the present invention, but it is desirably located immediately downstream of a structural gene.

Host cells include a microorganism belonging to Escherichia, Serratia, Bacillus, Brevibacterium, Corynebacterium, Microbacterium, Pseudomonas, Streptomyces and the like. Specific examples include Escherichia coli XL1-Blue, Escherichia coli XL2-Blue, Escherichia coli DH1, Escherichia coli MC1000, Escherichia coli KY3276, Escherichia coli W1485, Escherichia coli JM109, Escherichia coli HB101, Escherichia coli No.49, Escherichia coli W3110, Escherichia coli NY49, Escherichia coli G1698, Escherichia coli TB1, Serratia ficaria, Serratia fonticola, Serratia liquefaciens, Serratia marcescens, Bacillus subtilis, Bacillus amyloliquefacines, Brevibacterium ammoniagenes, Brevibacterium immariophilum ATCC14068, Brevibacterium saccharolyticum ATCC14066, Brevibacterium flavum ATCC14067, Brevibacterium lactofermentum ATCC13869, Corynebacterium glutamicum ATCC13032, Corynebacterium glutamicum ATCC 13869, Corynebacterium acetoacidophilum ATCC 13870, Microbacterium ammoniaphilum ATCC15354, Pseudomonas putida, Pseudomonas sp. D-0110, Streptomyces lividans TK23, Streptomyces lividans ATCC69411, Streptomyces coelicolor ATCC13405, Streptomyces griseus ATCC23915, Streptomyces avermitilis ATCC31267, Streptomyces avermitilis FERM BP-2773, and Streptomyces avermitilis FERM BP-2775, etc.

The recombinant vector may be introduced by any of the method for introducing DNA into the above host cells: for example, the method using calcium ion [Proc. Natl. Acad. Sci. USA, 69, 2110 (1972)], the protoplast method (Japanese Published Unexamined Patent Application No. 248394/88) and the method described in Gene, 17, 107 (1982) and Molecular & General Genetics, 168, 111 (1979).

When yeast is used as a host cell, examples of usable expression vector include YEP13 (ATCC37115), YEp24 (ATCC37051), YCp50 (ATCC37419), pHS19 and pHS15, etc.

Any promoter capable of functioning in yeast cells may be used, including glycolytic gene promoters such as hexose kinase, PHO5 promoter, PGK promoter, GAP promoter, ADH promoter, gal 1 promoter, gal 10 promoter, heat shock polypeptide promoter, MF α1 promoter and CUP 1 promoter.

Host cells include microorganisms belonging to Saccharomyces, Schizosaccharomyces, Kluyveromyces, Trichosporon, Schwanniomyces, Pichia and the Candida. Specific examples include Saccharomyces cerevisiae, Schizosaccharomyces pombe, Kluyveromyces lactis, Trichosporon pullulans, Schwanniomyces alluvius, or Candida utilis, etc.

The recombinant vector may be introduced by any of the method for introducing DNA into yeast: for example, electroporation [Methods Enzymol., 194, 182 (1990)], the spheroplast method [Proc. Natl. Acad. Sci. USA, 75, 1929 (1978)], the lithium acetate method [J. Bacteriology, 153, 163 (1983)] and the method described in Proc. Natl. Acad. Sci. USA, 75, 1929 (1978).

When an animal cell is used as a host cell, examples of usable expression vectors include pcDNAI, pcDM8 (manufactured by Funakoshi), pAGE107 [Japanese Published Unexamined Patent Application No. 22979/91, Cytotechnology, 3, 133 (1990)], pAS3-3 (Japanese Published Unexamined Patent Application No. 227075/90), pCDM8 [Nature, 329, 840 (1987)], pcDNAI/Amp (manufactured by Invitrogen), pREP4 (manufactured by Invitrogen), pAGE103 [J. Biochem., 101, 1307 (1987)], and pAGE210, etc.

Any promoter capable of functioning in animal cells may be used, including a promoter for immediate early (1E) gene of Cytomegalovirus (CMV), SV40 early promoter, retroviral promoter, metallothionein promoter, heat shock promoter, and SRapromoter. An enhancer for IE gene of Human CMV may also be used together with such a promoter.

Host cells include human Namalwa cells, monkey COS cells, chinese hamster CHO cells, or HBT5637 (Japanese Published Unexamined Patent Application No. 299/88).

The recombinant vector may be introduced into animal cells by any of the method for introducing DNA into animal cells: for example, electroporation [Cytotechnology, 3, 133 (1990)], calcium phosphate method (Japanese Published Unexamined Patent Application No. 227075/90), lipofection method [Proc. Natl. Acad. Sci. USA, 84, 7413 (1987)] and the method described in Virology, 52, 456 (1973), etc.

When an insect cell is used as a host cell, a polypeptide may be expressed by a method described in Current Protocols in Molecular Biology; Baculovirus Expression Vectors, A Laboratory Manual, W. H. Freeman and Company, New York (1992); or Bio/Technology, 6, 47 (1988).

More specifically, a recombinant gene-transfer vector and a baculovirus may be co-introduced into insect cells to obtain a recombinant virus in the supernatant from the culture of insect cells. Thereafter, insect cells may be further infected with the resulting recombinant virus to express the polypeptide.

A gene-transfer vector to be used in the above procedure includes pVL1392, pVL1393 and pBlueBacIII (manufactured by Invitrogen, respectively). As a baculovirus, for example, Autographa californica nuclear polyhedrosis virus, which infects Noctuidae insects, may be used.

Insect cells include Spodoptera frugiperda ovarian cells, Sf9 and Sf21, [Baculovirus Expression Vectors, A Laboratory Manual, W. H. Freeman and Company, New York (1992)], and Trichoplusia ni ovarian cells, High 5, (manufactured by Invitrogen), etc.

Co-introduction of the recombinant gene-transfer vector and the baculovirus into insect cells for recombinant virus production may be accomplished by the calcium phosphate method (Japanese Published Unexamined Patent Application No. 227075/90) or the lipofection method [Proc. Natl. Acad. Sci. USA, 84, 7413 (1987)].

When a plant cell is used as a host cell, examples of an expression vector include Ti plasmid and tobacco mosaic virus vector, etc.

Any promoter capable of functioning in plant cells may be used, including cauliflower mosaic virus (CaMV) 35S promoter and rice actin 1 promoter.

Host cells include plant cells such as tobacco, potato, tomato, carrot, soy bean, Brassica, alfalfa, rice, wheat and barley.

The recombinant vector may be introduced by any method for introducing DNA into plant cells: for example, Agrobacterium method (Japanese Published Unexamined Patent Application No. 140885/84, Japanese Published Unexamined Patent Application No. 70080/85, WO94/00977), electroporation method (Japanese Published Unexamined Patent Application No. 251887/85), and particle gun method (Japanese Patent No. 2606856, Japanese Patent No. 2517813).

The polypeptide of the present invention may be obtained by culturing a transformant of the present invention prepared as stated above in a medium until the polypeptide of the present invention is produced and accumulated in the culture, and collecting the polypeptide from the culture.

The transformant of the present invention may be cultured in a medium according to a conventional method used for culturing host cells.

When the transformant of the present invention is derived from a prokaryotic host such as Escherichia coli or a eukaryotic host such as yeast, the medium for culturing the transformant may be a natural or synthetic medium insofar as the medium contains a carbon source, a nitrogen source, inorganic salts etc., which can be assimilated by the transformant, and enables efficient culturing of the transformant.

Any carbon source assimilated by the transformant can be used. Examples include carbohydrates such as glucose, fructose, sucrose, molasses containing the same, starch and starch hydrolysates; organic acids such as acetic acid and propionic acid alcohols such as ethanol and propanol.

Examples of usable nitrogen source include ammonia, ammonium salts of inorganic or organic acids, such as ammonium chloride, ammonium sulfate, ammonium acetate, and ammonium phosphate; other nitrogen-containing compounds; and peptones, meat extracts, yeast extracts, corn steep liquor, casein hydrolysates, soy bean meal, soy bean meal hydrolysates, various fermented microorganism cells and hydrolysates thereof.

Inorganic salts usable herein include potassium dihydrogen phosphate, dipotassium hydrogen phosphate, magnesium phosphate, magnesium sulfate, sodium chloride, ferrous sulfate, manganese sulfate, copper sulfate, calcium carbonate, and the like.

Culturing is carried out under aerobic conditions as used for shaking culture or submerged aeration stirring culture. Culture temperature is preferably 15 to 40° C., and culture duration is usually for 16 hours to 7 days. During the culture, pH is preferably maintained at 3.0 to 9.0. pH is adjusted by using an inorganic or organic acid, an alkaline solution, urea, calcium carbonate, ammonia and the like.

If necessary, antibiotics such as ampicillin and tetracycline may be added to a medium during the culture.

Where a microorganism is transformed with a recombinant vector that contains inducible promoter, the transformant may be cultured in a medium supplemented with an inducer, if necessary. For example, in the case of a microorganism transformed with a recombinant vector comprising lac promotor, isopropyl-β-D-thiogalactopyranoside or the like may be add to the medium, and in the case of a microorganism transformed with a recombinant vector comprising trp promoter, indole acrylic acid or the like may be added.

A medium for culturing a transformant derived from an animal host cell may be a generally used medium such as RPMI 1640 medium [The Journal of the American Medical Association, 199, 519 (1967)], Eagle's MEM medium [Science, 122, 501 (1952)], Dulbecco's modified MEM medium [Virology, 8, 396 (1959)], 199 medium [Proceeding of the Society for the Biological Medicine, 73, 1 (1950)] or any one of these media further supplemented with fetal calf serum.

Culturing is usually carried out at pH 6 to 8, at a temperature of 30 to 40° C. for a period of 1 to 7 days in the presence of 5% CO₂.

If necessary, antibiotics such as kanamycin and penicillin may be added to the medium during the culture.

The medium for culturing a transformant derived from an insect host cell may be a generally used medium such as TNM-FH medium (manufactured by Pharmingen), Sf-900 II SFM medium (manufactured by Life Technologies), ExCell 400 and ExCell 405 [both manufactured by JRH Biosciences], Grace's Insect Medium [Nature, 195, 788 (1962)] or the like.

Culturing is carried out at pH 6 to 7, at a temperature of 25 to 30° C. for a period of 1 to 5 days.

If necessary, antibiotics such as gentamycin may be added to the medium during the culture.

The transformant derived from a plant host cell may be cultured as a cell or may be allowed to differentiate into plant cells or organs. The medium for culturing such a transformant may be a generally used medium such as Murashige and Skoog (MS) medium, White medium, or any one of these media further supplemented with a plant hormone such as auxin or cytokinin.

Culturing is usually carried out at pH 5 to 9, at a temperature of 20 to 40° C. for a period of 3 to 60 days.

If necessary, antibiotics such as kanamycin and hygromycin may be added to a medium during the culture.

As stated above, the polypeptide of the present invention may be obtained by culturing a microorganism-, animal cell-, or plant cell-derived transformant carrying a recombinant vector comprising a DNA that encodes the polypeptide in a general manner to produce and accumulate the polypeptide, and then recovering the polypeptide from the culture.

A gene of interest may be either expressed directly, or as a secretory protein or fusion polypeptide according to the method as described in Molecular Cloning, 2nd Edition.

Expression in yeast, animal, insect or plant cells can provide a polypeptide with sugar or sugar chain added thereto.

The protein of the present invention may be produced by intracellular production by host cells, extracellular secretion by host cells or production on outer membranes by host cells. Such production method can be selected depending on the kind of the host cells used or on alteration of the structure of the portein.

If the polypeptide of the present invention is produced in host cells or on the outer membranes of host cells, the polypeptide can be efficiently secreted extracellularly from the host cells by using the method of Paulson et al. [J. Biol. Chem., 264, 17619 (1989)], the method of Lowe et al. [Proc. Natl. Acad. Sci. USA, 86, 8227 (1989), Genes Develop., 4, 1288 (1990)] or methods as described in Japanese Published Unexamined Patent Application Nos. 336963/93 and 823021/94.

More specifically, the polypeptide of the present invention can be efficiently secreted from host cells by expressing it with a signal peptide, then using genetic recombination techniques, adding the signal peptide upstream of a polypeptide containing the active site of the polypeptide of the present invention.

Polypeptide production can be enhanced by utilizing a gene amplification system that uses a dihydrofolate reductase gene or the like according to the method described in Japanese Published Unexamined Patent Application No. 227075/90.

Further, animal or plant cells carrying a transgene may be re-differentiated to create an animal individual carrying a transgene (transgenic non-human animal) or a plant individual carrying a transgene (transgenic plant), which may be used for producing the polypeptide of the present invention.

When the transformant is an animal or plant individual, the polypeptide may be obtained by feeding or cultivating the individual in a general manner to produce and accumulate the polypeptide, and then recovering the polypeptide from the animal or plant individual.

In order to produce the polypeptide of the present invention using an animal individual, for example, an animal carrying a transgene may be allowed to produce therein the polypeptide of the present invention in a known manner as described in American Journal of Clinical Nutrition, 63, 639S (1996); American Journal of Clinical Nutrition, 63, 627S (1996); and Bio/Technology, 9, 830 (1991).

In the case of an animal individual, for example, the polypeptide of the present invention may be obtained by feeding a transgenic non-human animal carrying a DNA insert that encodes the polypeptide of the present invention to produce and accumulate therein the polypeptide, and then collecting the polypeptide from the animal. The polypeptide may be produced and accumulated in the animal's milk (Japanese Published Unexamined Patent Application No. 309192/88), egg and the like. Any promoter capable of functioning in an animal may be used, for example, mammary gland cell-specific promoters such as α-casein promoter, β-casein promoter, β-lactoglobulin promoter and whey acidic protein promoter being preferred.

In order to produce the polypeptide of the present invention using a plant individual, for example, a transgenic plant carrying a DNA insert encoding the polypeptide of the present invention may be cultivated to produce and accumulate therein the polypeptide in a known manner as described in Tissue Culture (Soshiki Baiyo), 20 (1994); Tissue Culture, 21 (1995); and Trends in Biotechnology, 15, 45 (1997), and then the polypeptide may be recovering from the plant.

For isolation and purification of the polypeptide produced from the transformant of the present invention, conventional methods for the isolation and purification of enzymes can be used.

For example, if the polypeptide of the present invention is expressed in a soluble form in cells, after completion of culturing, the cells are collected by centrifugation, suspended in an aqueous buffer and then disrupted with ultrasonic disrupter, French Press, Manton-Gaulin homogenizer, Dynomill or the like, thereby obtaining a cell-free extract. A purified preparation can be obtained by centrifuging the cell-free extract. The obtained supernatant is then subjected to conventional isolation and purification methods for enzymes, i.e., solvent extraction, salting-out or desalting with sulfate ammonium etc., precipitation with organic solvent, anion-exchange chromatography on resin such as diethylaminoethyl (DEAE)-Sepharose or DIAION HPA-75 (manufactured by Mitsubishi Chemical Industries Ltd.), cation-exchange chromatography on resin such as S-Sepharose FF (manufactured by Pharmacia), hydrophobic chromatography on resin such as butyl Sepharose or phenyl Sepharose, gel filtration using molecular sieve, affinity chromatography, chromatofocusing, or electrophoresis such as isoelectric focusing, or combinations thereof.

If the polypeptide is expressed as inclusion body in cells, the cells are similarly collected, disrupted and centrifuged to give an insoluble matter of the polypeptide as a precipitated fraction. The resulting insoluble polypeptide is then solubilized with a protein-denaturing agent. The solubilized solution is then diluted or dialyzed to reduce the agent to a lower concentration, thereby allowing the polypeptide to be renatured to its normal conformation. The purified preparation of the polypeptide can be then obtained by use of the same isolation and purification methods as described above.

If the polypeptide of the present invention or a derivative thereof having a sugar chain added thereto is extracellularly secreted, the polypeptide or its derivatives may be recovered in the culture supernatant. Namely, the culture is subjected to the same process, such as centrifugation, as described above to give a culture supernatant. From the culture supernatant, a purified preparation can be obtained in the same manner for isolation and purification as described above.

The polypeptide thus obtained may be, for example, a polypeptide having the amino acid sequence shown in SEQ ID NO: 8.

The polypeptide of the present invention may be produced by chemical synthesis methods including Fmoc method (fluorenyl methyloxycarbonyl method), t-Boc method (t-butyloxycarbonyl method), and so on. Also, it may be chemically synthesized using a peptide synthesizer available from Advanced ChemTech, Perkin Elmer, Pharmacia, Protein Technology Instrument, Synthecell-Vega, PerSeptive or Shimadzu Corporation, etc.

In contrast, a method for inserting DNA having mutation which has been introduced in vitro into the chromosomal DNA of the host cell can be carried out by any method utilizing the homologous recombination of DNA. Examples of such methods include a method described in Japanese Published Unexamined Patent Application No. 189774/94.

Cells having a modified avermectin aglycon synthase gene having mutation introduced as described above are not particularly limited insofar as cells can carry the gene and may be any prokaryotic cells such as Escherichia coli, Bacillus subtilis, and Actinomyces. Examples thereof include microorganisms belonging to Streptomyces avermitilis.

[3] Preparation of Substrate Compound for Producing 22,23-dihydroavermectin B1a or Derivative Thereof.

In the present invention, the substrate compound for producing 22,23-dihydroavermectin B1a or a derivative thereof may be any substance insofar as the substance can be used as a substrate for the modified avermectin aglycon synthase as described above. More specifically, in the process for synthesizing avermectin aglycon, the substance can be a substrate for the domain responsible for the later reaction step in the modified domain and an N-acetylcysteamine compound is preferably used. For example, when the KS domain of SU1 shown in FIG. 2 is modified, the N-acetylcysteamine compound preferably has a structure as represented by formula (I):

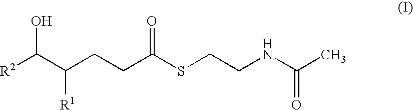

wherein R¹ and R², which may be the same or different, represent hydrogen, substituted or unsubstituted alkyl, substituted or unsubstituted alkenyl, substituted or unsubstituted aryl, or substituted or unsubstituted heterocycle, or, R¹ and R² together form, substituted or unsubstituted cycloalkyl.

In defining each group in formula (I), examples of alkyl include straight chain or branched C₁₋₂₀ methyl, ethyl, propyl, isopropyl, butyl, sec-butyl, tert-butyl, pentyl, isopentyl, neopentyl, hexyl, heptyl, decyl, dodecyl, pentadecyl, and eicosyl, etc.

Examples of alkenyl include straight chain or branched C₂₋₂₀ vinyl, allyl, 1-propenyl, methacryl, chrotyl, 1-butenyl, 3-butenyl, 2-pentenyl, 4-pentenyl, 2-hexenyl, 5-hexenyl, heptenyl, decenyl, dodecenyl, pentadecenyl, and eicosenyl, etc.

Examples of aryl include C₆₋₁₄ phenyl, naphthyl, and anthryl, etc.

Examples of heterocycle include aromatic heterocycle such as pyridyl, pyrazinyl, pyrimidinyl, pyridazinyl, quinolinyl, isoquinolinyl, phthalazinyl, quinazolinyl, quinoxalinyl, naphthylizinyl, cinnolinyl, pyrrolyl, pyrazolyl, imidazolyl, triazolyl, tetrazolyl, thienyl, furyl, thiazolyl, oxazolyl, indolyl, indazolyl, benzimidazolyl, benzotriazolyl, benzothiazolyl, benzoxazolyl, and purinyl; and alicyclic heterocycle such as pyrrolidinyl, piperidino, piperazinyl, morpholino, thiomorpholino, homopiperidino, homopiperazinyl, tetrahydropyridinyl, tetrahydroquinolinyl, tetrahydroisoquinolinyl, tetrahydrofuranyl, tetrahydropiranyl, and dihydrobenzofuranyl, etc.

Examples of cycloalkyl include C₃₋₈ cyclopropyl, cyclobutyl, cyclopentyl, cyclohexyl, cycloheptyl, and cyclooctyl, etc.

Substituted alkyl, substituted alkenyl, and substituted cycloalkyl may be mono-, di-, tri-substituted and each substituent is the same or different. Example of substituents include hydroxy and substituted or unsubstituted alkoxy. The alkyl portion of alkoxy has the same meaning as the above alkyl and substituted alkoxy may be mono-, di-, tri-substituted by, for example, hydroxy.

Substituted aryl and substituted heterocycle may be mono-, di-, tri-substituted and each substituent is the same or different. Example of substituents include hydroxy, substituted or unsubstituted lower alkyl, and substituted or unsubstituted lower alkoxy, etc. The lower alkyl and lower alkoxy have the same meaning as the above and substituted lower alkyl and substituted lower alkoxy may be mono-, di-, tri-substituted by, for example, hydroxy.

Specific examples of such compounds include a compound (Compound 4 shown in the table below) represented by the above formula, wherein R¹ is methyl and R² is sec-butyl. The compound employs, for example, Compound A shown in the table below as a starting material and can be chemically synthesized in the following manner through Compounds 1 to 3 similarly shown in the table.

At the outset, Compound 1 is prepared using Compound A as a starting material and performing ozone oxidation, followed by the Wittig reaction to add carbon chains. After t-butyldimethylsilyl of Compound 1 is deprotected, a protective group is reintroduced using chloroethyl-tri-silane to obtain Compound 2. Subsequently, α-β unsaturated carbon bond in compound 2 is reduced in the presence of a palladium-carbon catalyst, ester is hydrolyzed with potassium hydroxide and neutralized, followed by the addition of N-acetylcysteamine in the presence of a condensing agent. Thus, a thioester compound, Compound 3, is obtained. Finally, acetic acid is added to Compound 3 to remove the protective group. Thus, Compound 4 is prepared.

Other compounds represented by formula (I) can also be produced in the same manner.

The intermediates and the compounds of interest in the above production method are subjected to separation purification methods, which are commonly used in organic synthetic chemistry, for example, filtration, extraction, washing, drying, concentration, recrystallization, or various chromatographies and, thus, they can be isolated and purified. The intermediate can be applied to the subsequent reaction without purification.

TABLE 1
Compounds

| A | 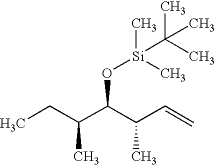 |
|---|---|
| 1 | 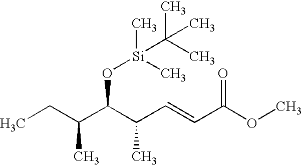 |
| 2 | 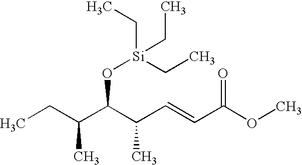 |
| 3 | 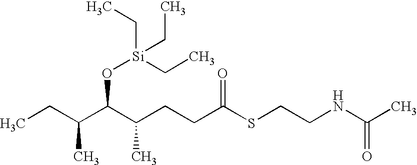 |
| 4 | 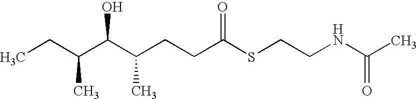 |

[4] Production of 22,23-dihydroavermectin B1a or Derivative Thereof.

Any of the culture, cells or treated cells of the cells obtained by transforming the host cell in [2]-2 can be used in the reaction with the substrate compounds so far as the modified avermectin aglycon expressed in the transformed cell are functioned.

Treated cells include dried cells, freeze-dried products, surfactant- or organic solvent-processed products, enzyme-processed products, ultrasonicated products, mechanically ground products, protein fractions of cells, and immobilized cells of treated cells.

Any method of making the substrate compound acting upon the transformed host cell can be used so far as the synthesis of avermectin aglycon is disturbed. Specific examples thereof include a method in which the culture of cells or treated products thereof are reacted with the substrate in a suitable medium and a method in which the cells are cultured by adding the substrate in initially or mid course of the culturing.

Media used in the reaction include water, buffers such as phosphate, carbonate, acetate, borate, citrate and Tris, aqueous solutions containing organic solvents, for example, alcohols such as methanol and ethanol, esters such as ethyl acetate, ketones such as acetone, and amides such as acetamide. If necessary, surfactants such as Triton X-100 (manufactured by Nacalai Tesque, Inc.) or Nonion HS204 (manufactured by NOF Corp.) or organic solvents such as toluene and xylene may be added in an amount of about 0.1 to 20 g/l.

Reaction is carried out in the above aqueous solution at pH 5 to 10, preferably pH 6 to 8, at 20 to 50° C. for 1 to 96 hours.

When the host cell is cultured in a medium, culture can be carried out in the same manner as for obtaining the polypeptide.

22,23-dihydroavermectin B1a or a derivative thereof can be isolated from the reaction product or the culture obtained by any of the above methods in accordance with conventional isolation methods. For example, the cultured cell is treated with acetone or methanol to extract 22,23-dihydroavermectin B1a or a derivative thereof and, after the removal of the residue, concentrated. The concentrate is processed with methylene chloride, the methylene chloride layer is fractionated and further concentrated under reduced pressure. Thus, the subject compound can be obtained.

### BRIEF DESCRIPTION OF DRAWINGS

FIG. 1 is a diagram showing a restriction map of BamHI, BglII, ClaI, EcoRI, KpnI, Mlul, PstI, StuI, and XhoI sites of avermectin aglycon synthase genes aveAI and aveAII of Streptomyces avermitilis. The arrows indicate the deduced transcription direction of each gene.

FIG. 2(a) shows the location of avermectin aglycon synthase genes on the chromosome and the domain sequence of synthase units, FIGS. 2(b) and 2(c) show the deduced steps of avermectin aglycon synthesis, and FIG. 2(d) shows the structure of 6,8-sec-6,8a-deoxy-5-oxoavermectin aglycon and the location of integrated lower fatty acids in its skeleton which had been synthesized by a polyketide synthase, which is a gene product of avermectin aglycon synthase genes aveAI and aveAII.

(Description of Reference Characters)

ACP: acyl carrier protein

KS: β-ketoacyl ACP synthase

AT: acyltransferase

KR: β-ketoacyl ACP reductase

DH: dehydratase

ER: enoyl reductase

TE: thioesterase

FIG. 3 is a diagram showing a procces for constructing a plasmid to be used in the transformation of Streptomyces avermitilis wherein (I) shows plasmid pKS1 prepared by cloning KS1 containing DNA encoding an amino acid residue in an active center, (II) shows plasmid pKSmut prepared by cloning DNA encoding KS1 prepared by substituting an amino acid residue in an active center, (III) shows plasmid pKSmutRL prepared by applying addition and substitution of a DNA fragment shown in (IV) to pKSmut, and (IV) is the restriction map of DNA encoding KS1 used in the construction of pKSmutRL.

In the drawing, “ [symbol] ” indicates the location of the nucleotides which have been substituted, and HindIII, PstI, BamHI, KpnI, and EcoRI indicate the DNA cleavage sites of each restriction enzyme. Numerical values in (I), (II), and (III) indicate, when a desired nucleotide of each plasmid is determined as No. 1, the distance (bp) from the nucleotide and numerical values with in the circle indicate the total plasmid length (bp). Numerical values in (IV) are in accordance with the nucleotides shown in SEQ ID NO: 1. Abbreviations in the drawings are as follows.

(Description of Reference Characters)

bla: β-lactamase (arrow indicates the direction of transcription)

ori: replication origin (origin)

Plac: β-lactamase promoter (arrow indicates the direction of the promoter)

IG: M13 phage intergenic region (M13 Intergenic region)

### BEST MODES FOR CARRYING OUT THE INVENTION

The present invention will be described in more detail with reference to examples; however, these examples are not intended to limit the scope of the present invention.

### EXAMPLE 1

### Determination of Nucleotide Sequence and Structure of Avermectin Aglycon Synthase Gene

A nucleotide sequence of DNA encoding avermectin aglycon synthase derived from Streptomyces avermitilis K2033 (U.S. Pat. No. 5,206,155, FERM BP-2773) was determined as follows.

A continuous or overlapping DNA fragment within the avermectin aglycon synthase gene was subcloned from a cosmid containing fragments of the avermectin aglycon synthase genes (aveAI and aveAII) co-isolated with a gene encoding avermectin B5-O-transmethylase [aveD; Gene, 206, 175-180 (1998)]. Nucleotide sequences of the inserted DNA fragments in these subclones were then determined.

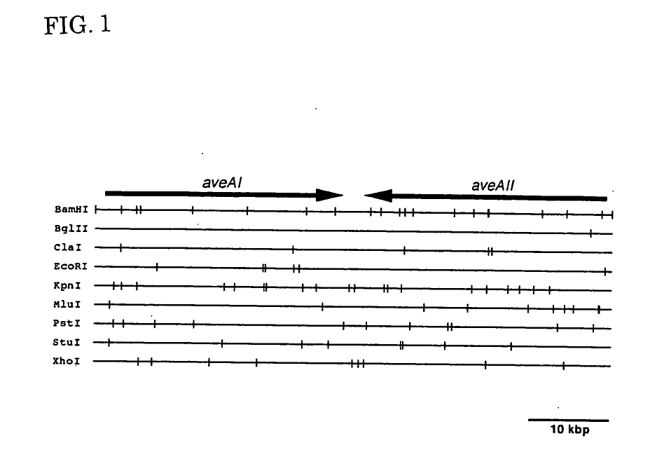

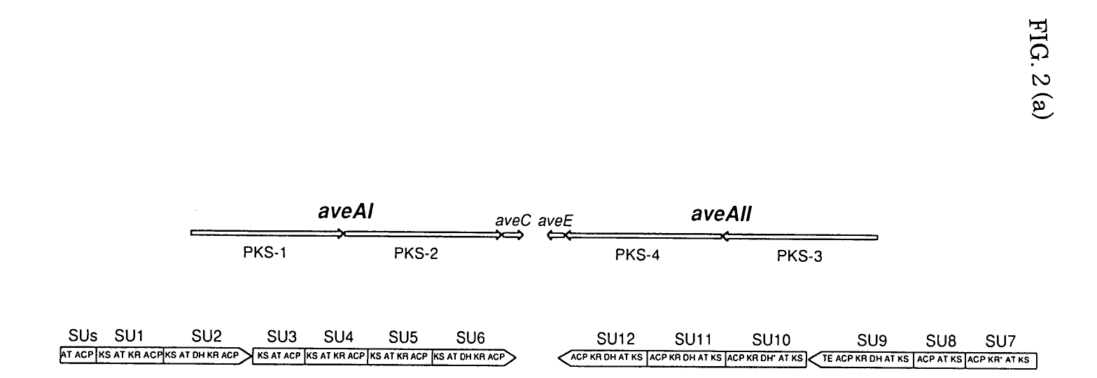

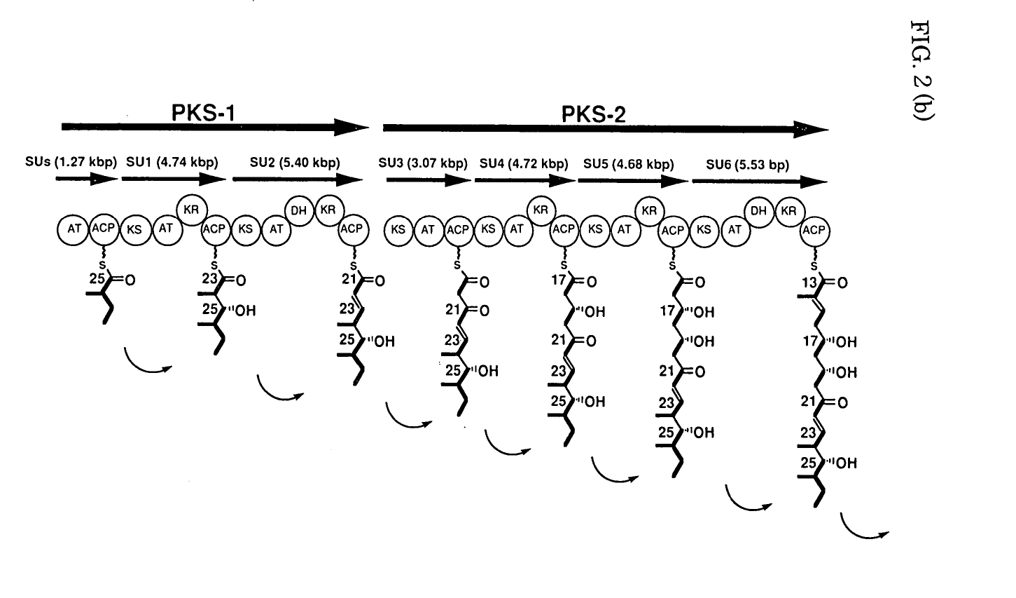

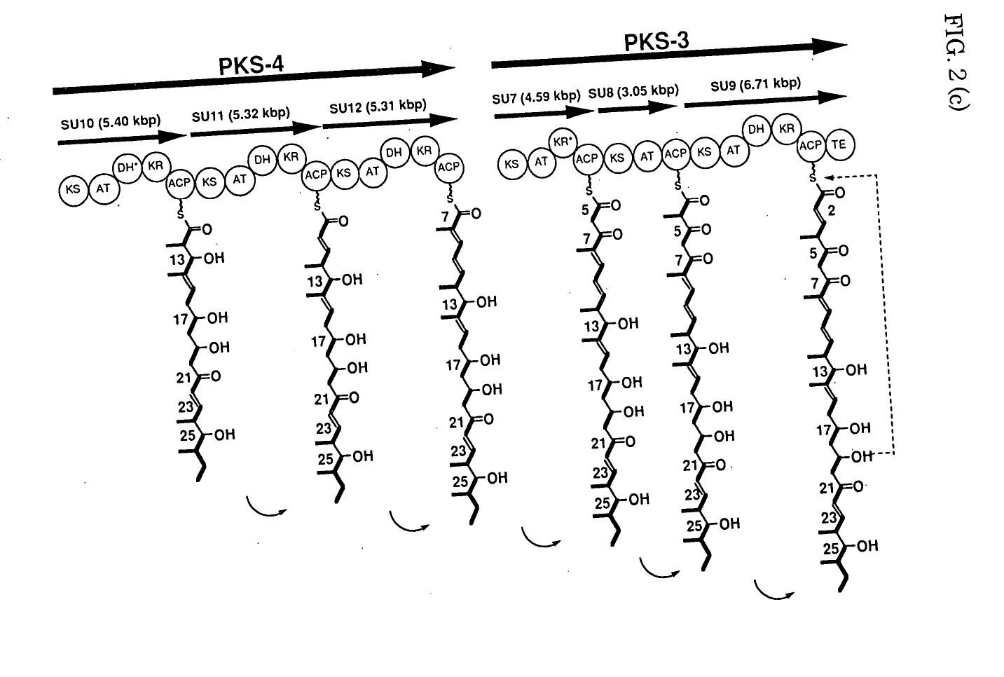

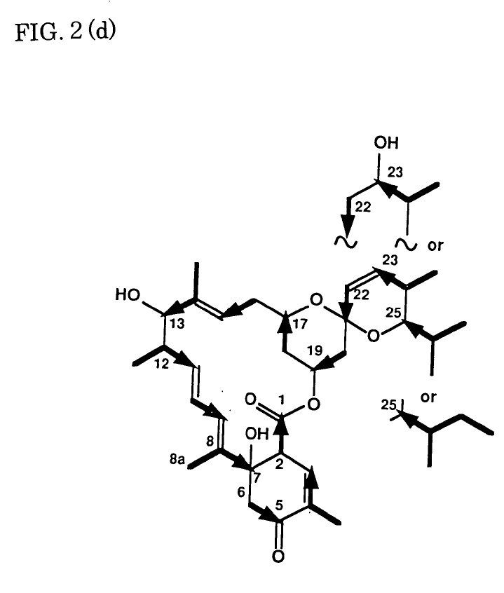

More specifically, the entire nucleotide sequences of aveAI and aveAII were determined by subcloning BamHI-digested fragments of 3.4 kbp, 2.0 kbp, 0.5 kbp, 6.8 kbp, 7.0 kbp, 7.8 kbp, 3.7 kbp, 4.8 kbp, 1.3 kbp, 2.4 kbp, 0.7 kbp, 1.0 kbp, 5.4 kbp, 2.5 kbp, 1.9 kbp, 0.1 kbp, 7.0 kbp, 3.1 kbp, 4.7 kbp and 1.3 kbp found in the BamHI-restriction map of aveAI and aveAII shown in FIG. 1; digesting the inserted DNA fragments in these subclones with exonuclease III and S1 nuclease to prepare a series of deletion fragments; and then performing a cycle-sequencing reaction using fluorescently-labeled primers to determine a nucleotide sequence of each deleted fragment. aveAI and aveAII had the nucleotide sequences shown in SEQ ID NO: 1 and SEQ ID NO: 2, respectively.

### EXAMPLE 2

### Preparation of Strain Applied for the Direct Production of 22,23-dihydroavermectin B1a

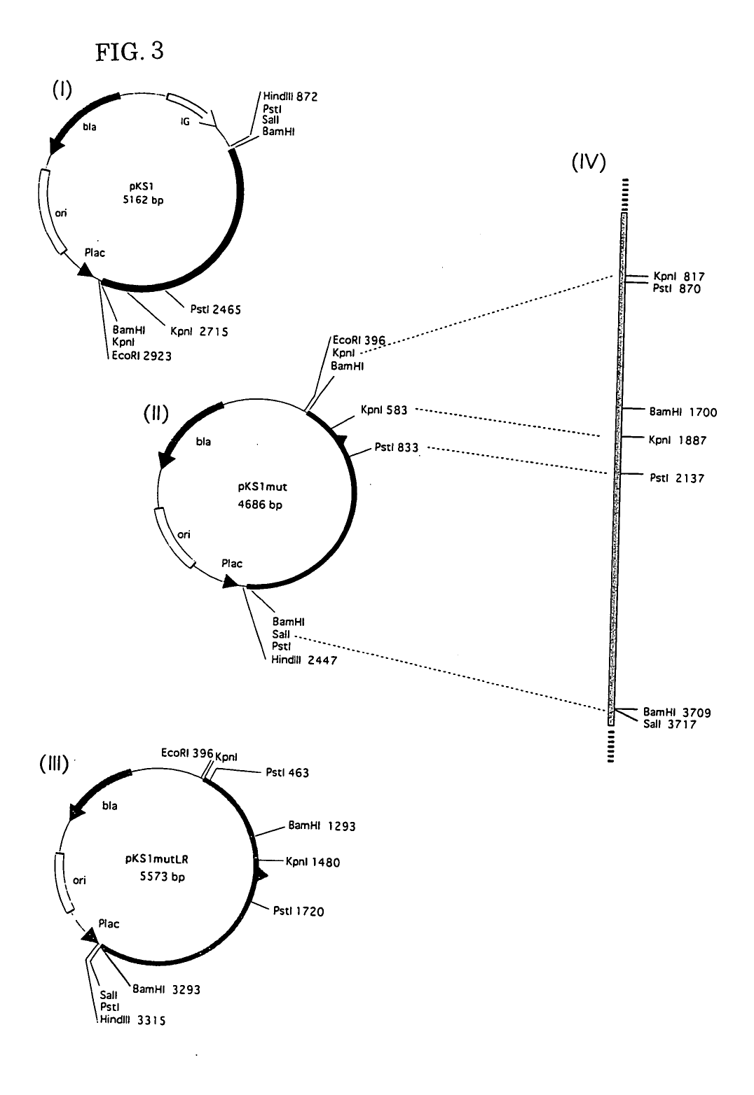

The plasmid shown in FIG. 3 was produced in accordance with the following method and used in the transformation of Streptomyces avermitilis.

(1) Subcloning of a DNA Fragment Containing KS1

The cosmid DNA containing KS1, from among cosmid DNAs containing avermectin aglycon synthase genes, was digested with the restriction enzyme BamHI (manufactured by Takara Shuzo Co., Ltd.) followed by agarose gel electrophoresis (described in Molecular Cloning, 2nd Edition), and 2.0 kb DNA fragment (see FIG. 1, 1701 to 3716 shown in SEQ ID NO: 1) containing a cysteine residue (amino acid 657 shown in SEQ ID NO: 4), which is an active center of KS1, was separated and purified in accordance with the method described in Molecular Cloning, 2nd Edition. Plasmid pUC118 (manufactured by Takara Shuzo Co., Ltd.) was digested with BamHI and dephosphorylated with alkaline phosphatase from calf intestine (manufactured by Takara Shuzo Co., Ltd.). About 0.1 μg each of 2.0 kb DNA fragment containing KS1 and a BamHI digested pUC118 were ligated 16° C. for 16 hours using Ligation High (manufactured by Toyobo Co., Ltd.). 10 μl of this DNA ligation reactant was brought into contact with a competent cell of Escherichia coli DH5a (manufactured by Nippon Gene Co., Ltd.) and transformed in accordance with the method described in Molecular Cloning, 2nd Edition. In selecting the transformant, an LB agar medium containing 50 μg/ml ampicillin (manufactured by Wako Pure Chemical Industries, Ltd.) was used. 50 μl of aqueous solution of 0.1 mol/l isopropyl-β-D-thiogalactopyranoside (IPTG, manufactured by Wako Pure Chemical Industries, Ltd.) and 50 μl of 2% solution of 5-bromo-4-chloro-3-indolyl-β-D-galactoside (X-gal, manufactured by Nacalai Tesque, Inc.) in dimethylformamide (manufactured by Nacalai Tesque, Inc.) were previously spread on the 20 ml of LB agar medium. The colony of the transformant carrying the recombinant plasmid has lost its β-galactosidase activity, and thus, cannot decompose 5-bromo-4-chloro-3-indolyl-β-D-galactoside while developing white color. This white colony was collected with the aid of ase, inoculated on 10 ml of LB medium, and subjected to shaking culture at 37° C. for 16 hours. The plasmid was then extracted from the cells and purified in accordance with the alkaline method described in Molecular Cloning, 2nd Edition. A part of the resulting recombinant plasmid was digested with a restriction enzyme PstI and it was confirmed that plasmid pKS1, into which a DNA fragment containing KS1 genes was inserted in the same direction with lacZ encoded by pUC118, was obtained.

(2) Introduction of Nucleotide Substitution into the Active Center of KS1

Nucleotide was substituted using Takara LA PCR in vitro Mutagenesis Kit (manufactured by Takara Shuzo Co., Ltd.). Nucleotide was hereinafter substituted in accordance with the protocol attached to the kit. The recombinant plasmid containing KS1 genes prepared in (1) above was used as template DNA for the 1st PCR. As a primer for the 1st PCR-(a), 5′-ACCGTGGACACGGGGGGCTCGGCATCGCTCGT-3′ shown in SEQ ID NO: 9 (corresponding to 1954 to 1985 shown in SEQ ID NO: 1, “T” at the 1969 position was substituted with “G”) and M13M4 primer (attached to the kit) were used as a primer for introducing mutation. M13RV primer and MUT4 primer (attached to the kit) were used as primers for the 1st PCR-(b). In the 1st PCR, incubation at 98° C. for 5 minutes, and then 30 cycles of reaction constituted by 30 seconds at 94° C., 2 minutes at 55° C. and 3 minutes at 72° C. as one cycle were carried out. TaKaRa PCR Thermal Cycler 480 (manufactured by Takara Shuzo Co., Ltd.) was used in PCR. Each reaction solution was subjected to agarose gel electrophoresis and about 1.8 kb amplified fragment in the 1st PCR-(a) and about 2.0 kb amplified fragment in the 1st PCR-(b) were respectively separated and purified for use in the subsequent step. Heteroduplex DNA between amplified fragments obtained in the 1st PCR was formed by incubating at 98° C. for 15 minutes, lowering the temperature to 37° C. over the course of 1 hour, and then incubating at 37° C. for 15 minutes. After LA Taq polymerase was added to the reaction solution, the mixture was incubated at 72° C. for 3 minutes to convert the terminus of the heteroduplex DNA into a blunt-ended terminus. In the subsequent 2nd PCR, 30 cycles of reaction constituted by 20 seconds at 94° C., 30 seconds at 60° C. and 3 minutes at 72° C. as one cycle were carried out. A part of the 2nd PCR product was subjected to agarose gel electrophoresis and the amplification of about 2.0 kb fragment was confirmed. The remaining solution of the 2nd PCR was thoroughly mixed with a phenol:chloroform=1:1 solution saturated with water and then centrifuged. The supernatant was subjected to ethanol precipitation in accordance with the method described in Molecular Cloning, 2nd Edition, dried, and then redissolved in water. Restriction enzymes HindIII and EcoRI (manufactured by Takara Shuzo Co., Ltd.) were added to the DNA solution and the DNA was digested. Agarose gel electrophoresis was subsequently performed, thereby separating and purifying 2.0 kb DNA fragment. Plasmid vector pUC19 (manufactured by Takara Shuzo Co., Ltd.) was also digested with HindIII and EcoRI. 2.7 kb fragment was then separated and purified by agarose gel electrophoresis. 2.0 kb DNA fragment digested with HindIII and EcoRI was ligated to pUC19 using Ligation High and used to the transformation of Escherichia coli DH5a. As with (1) above, IPTG and X-gal were spread on the LB agar medium containing 50 μg/ml ampicillin for the selection of the transformant. Several strains were selected among from the transformants obtained as white colonies and inoculated on 10 ml of LB medium containing 50 μg/ml ampicillin and subjected to shaking culture at 37° C. for 16 hours. Thereafter, strains were harvested and plasmid DNA carried by each strain was extracted and purified by an alkaline method.

(3) Confirmation of Introduction of Nucleotide Substitution by Nucleotide Sequencing

In nucleotide sequencing, ABI PRISM DNA Sequencing Kits-Dye primer Cycle Sequencing Ready Reaction Kits with AmpliTaqR DNA Polymerase, FS-21M13-(manufactured by PE Applied Biosystems), and ABI373A were used. Each recombinant plasmid DNA, which is considered to have nucleotide substitution introduced KS1 obtained in (2) above, was used as templates and sequencing samples were produced by PCR in accordance with the protocol attached to the Sequencing Kits. Each sample was subjected to electrophoresis using ABI373A and the resultant data was analyzed using a software for gene analysis, Genetyx (manufactured by Software Development Co., Ltd.). As a result, it was confirmed that plasmid DNA (pKS1mut) containing about 2.0 kb BamHI fragment corresponding to 1701 to 3716 in SEQ ID NO: 3 was obtained. SEQ ID NO: 3 comprises a nucleotide sequence in which thymine at the 1969 position is substituted with guanine in the 1^{st} to 11916^{th} nucleotide sequences shown in SEQ ID NO: 1.

(4) Introduction of Nucleotide Substitution into Chromosomal DNA of Streptomyces avermitilis

In order to introduce the plasmid mutation into chromosomal DNA through homologous recombination, a reasonably long homologous region is necessary. Since mutation is introduced into the DNA by PCR, mutation may be introduced in the region other than the targeted site. Thus, the broadest possible region other than the mutation site should be substituted with DNA derived from chromosomal DNA of Streptomyces avermitilis to eliminate unnecessary mutation. Plasmid DNA used in the homologous recombination was constructed in the following manner and applied to the transformation of Streptomyces avermitilis.

pKS1mut produced in (3) above was digested with restriction enzymes PstI and SalI (manufactured by Takara Shuzo Co., Ltd.) and then subjected to agarose gel electrophoresis to separate and purify 4.1 kb DNA fragment. Subsequently, pKS1 was digested with PstI and SalI, followed by electrophoresis and 1.57 kb PstI and SalI digested fragments were separated and purified. Each collected DNA fragment was ligated using Ligation High and then brought into contact with a competent cell of Escherichia coli DH5α for transformation. The transformant was selected using LB agar medium containing 50 μg/ml ampicillin. Transformants were cultured at 37° C. for 16 hours and ten-odd colonies were collected with the aid of ase, inoculated on 10 ml of LB medium containing 50 μg/ml ampicillin, subjected to shaking culture at 37° C. for 16 hours, harvested, and plasmid carried by each strain was purified by the alkaline method. Each plasmid was digested with restriction enzymes PstI and SalI, subjected to agarose gel electrophoresis, and it was confirmed that plasmid pKS1mutR containing 4.1 kb and 1.57 kb DNA fragments was obtained.

Subsequently, pKS1mutR was digested with restriction enzyme KpnI (manufactured by Takara Shuzo Co., Ltd.) and treated with alkaline phosphatase. Then, a cosmid, which contains a KpnI region represented by nucleotide 817 to 1887 shown in SEQ ID NO: 1, was digested with KpnI, followed by electrophoresis, and about 1.1 kb KpnI fragment was separated and purified. Each purified DNA fragment was ligated using Ligation High and then brought into contact with a competent cell of Escherichia coli DH5α for transformation. The transformant was selected using the LB agar medium containing 50 μg/ml ampicillin. Transformants were cultured at 37° C. for 16 hours and ten-odd colonies were collected with the aid of ase, inoculated on 10 ml of LB medium containing 50 μg/ml ampicillin, subjected to shaking culture at 37° C. for 16 hours, harvested, and plasmid carried by each strain was purified by the alkaline method. Each plasmid was digested with restriction enzyme PstI, subjected to agarose gel electrophoresis, and it was confirmed that plasmid pKS1mutRL containing 1.27 kb, 1.57 kb, and 2.7 kb DNA fragments was obtained.

Subsequently, pKS1mutRL was digested with restriction enzymes HindIII and EcoRI and 2.9 kb HindIII and EcoRI DNA fragments were separated and purified by agarose gel electrophoresis. Plasmid vector pKC7 (Japanese Published Unexamined Patent Application No. 189774/94) was also digested with HindIII and EcoRI and then purified by agarose gel electrophoresis. These two DNA fragments were ligated at 16° C. for 16 hours using Ligation High and then brought into contact with a competent cell of Escherichia coli DH5α for transformation. Transformants were selected using the LB agar medium containing 50 μg/ml ampicillin. Those transformants were cultured at 37° C. for 16 hours and ten-odd colonies were collected with the aid of ase, and inoculated on 10 ml of LB medium containing 50 μg/ml ampicillin. Those transformants were cultured at 37° C. for 16 hours, and then cells were harvested and plasmid carried by each strain was purified by the alkaline method. Each plasmid was digested with restriction enzymes HindIII and EcoRI and then subjected to agarose gel electrophoresis. Thus, it was confirmed that plasmid pKC-KS1mut carrying 2.9 kb fragment was obtained.

KS1mut fragment was integrated into the KS1 region of the chromosome of Streptomyces avermitilis K2038 (FERM BP-2775) by homologous recombination using pKC-KS1mut in accordance with the method described in Japanese Published Unexamined Patent Application No. 189774/94. In order to confirm that KS1mut was replaced on the chromosomal DNA, the chromosomal DNA of the thus obtained recombinant strain was prepared by the method described in Japanese Published Unexamined Patent Application No. 189774/94, and PCR was carried out using the chromosomal DNA as a template and using the synthetic DNA shown in SEQ ID NO: 10 (5′-ATAAGCTTAATCGATCCGCTGTCCGGTA-3′, containing a sequence corresponding to nucleotides 1758 to 1776 in SEQ ID NO: 1) and the synthetic DNA shown in SEQ ID NO: 11 (5′-ATGAATTCCCTCCAAAATCACATGCGCATT-3′, containing a sequence corresponding to nucleotides 2710 to 2729 in SEQ ID NO: 1) as a primer set. The about 1.0 kb amplified DNA fragment was digested with restriction enzymes HindIII and EcoRI and about 1.0 kb amplified fragment was then separated and purified by agarose gel electrophoresis. Plasmid vector pUC19 was also digested with restriction enzymes HindIII and EcoRI and then separated and purified by agarose gel electrophoresis. The two DNA fragments thus obtained were ligated at 16° C. for 16 hours using Ligation High and then used to the transformation of Escherichia coli DH5α. IPTG and X-gal were spread on the LB agar medium containing 50 μg/ml ampicillin for selecting the transformant. Several strains were selected among from the transformants, obtained as white colonies, and inoculated on 10 ml of LB medium containing 50 μg/ml ampicillin. After the transformants were cultured by shaking, cells were harvested and plasmid carried by each strain was extracted and purified by the alkaline method. The thus obtained plasmid was used to determine the nucleotide sequence in the manner as described in (3) above. Thus, it was confirmed that the subject recombinant Streptomtces avermitilis KS1mut strain was obtained.

### EXAMPLE 3

### Synthesis of Substrate Compound

Physicochemical data of the following compounds were measured using the following instruments.

| | | |
|---|---|---|
| MS | JEOL. Ltd | HX/HX110A |
| ¹H NMR | JEOL. Ltd | Lambda 300 (300 MHz) |

In the physical data of the compounds, “FABMS” indicates the mass spectrum obtained by the “FAB” method. The term “conventional post-processing” refers to processing after the reaction.

After the completion of the reaction in each step, water, acids, buffers or the like is optionally added to the reaction solution to extract with a non-aqueous solvent such as ethyl acetate, ether, chloroform, and dichloromethane. The extract is washed with water, a saline solution, etc. and then dried over anhydrous sodium sulfate, thereby removing the solvent by distillation under reduced pressure.

(1) Synthesis of Compound 1

Compound A (16 g, 0.060 mol; Table 1) was dissolved in methanol (620 mL) and ozone-air current was blown at −78° C. while stirring for 4 hours. After air was blown into the reaction solution for 15 minutes, dimethylsulfide (44 mL, 0.60 mol) was added thereto, and the mixture was stirred at 25° C. for 15 hours. After the conventional post-processing, the residue was dissolved in toluene (290 mL), methyl (triphenylphosphoranylidene) acetate (33.7 g, 0.10 mol) was added, and the mixture was stirred at 65° C. for 17 hours. After conventional post-processing, purification was carried out by chromatography on silica gel (eluted at hexane/ethyl acetate=100/0 to 10/1) to give Compound 1 (9.4 g, yield 53%; Table 1).

¹H NMR (CDCl₃) δ ppm; 7.04 (dd, J=8.3, 15.8 Hz, 1H), 5.78 (dd, J=1.1, 15.7 Hz, 1H), 3.72 (s, 3H), 3.48 (t, J=3.5 Hz, 1H), 2.52 (m, 1H), 1.35-1.54 (m, 2H), 1.10 (m, 1H), 1.04 (d, J=7.0 Hz, 3H), 0.40 (s, 9H), 0.37 (d, J=7.4 Hz, 3H), 0.35 (d, J=6.8 Hz, 3H), 0.03(s, 3H), 0.02 (s, 3H)

FABMS: M/Z 315 (M+H)⁺

Molecular formula-based theoretical value: C₁₇H₃₄N₃Si=314

(2) Synthesis of Compound 2

Compound 1 (0.20 g, 0.63 mmol) was dissolved in methanol (8.9 mL) and 10% hydrogen chloride/methanol solution (0.99 mL) was added thereto, and the mixture was stirred at 50° C. for 1 hour. After conventional post-processing, the residue was dissolved in N,N-dimethylformamide (6.2 mL), chlorotritylsilane (0.31 mL, 1.8 mmol) and imidazole (0.21 g, 3.1 mmol) was added thereto, and the mixture was stirred at 25° C. for 1.5 hours. After conventional post-processing, purification was carried out by chromatography on silica gel (eluted at hexane/ethyl acetate=25/1) to give Compound 2 (0.18 g, yield 93%; Table 1).

¹H NMR (CDCl₃) δ ppm; 7.04 (dd, J=8.4, 15.7 Hz, 1H), 5.79 (dd, J=1.1, 15.7 Hz, 1H), 3.73 (s, 3H), 3.48 (dd, J=4.1, 5.4 Hz, 1H), 2.51 (m, 1H), 1.35-1.51 (m, 2H), 1.12 (m, 1H), 0.81-1.08 (m, 18H), 0.47-0.66 (m, 6H)

FABMS: m/z 315 (M+H)⁺

Molecular formula-based theoretical value: C₁₇H₃₄N₃Si=314

(3) Synthesis of Compound 3

Compound 2 (4.1 g, 0.013 mol) was dissolved in ethanol (200 mL), 10% palladium-carbon (0.41 g) was added thereto, and the mixture was stirred under hydrogen atmosphere at 25° C. for 4.5 hours. After the reaction solution was passed through Celite R545, the solvent was removed by distillation under reduced pressure. The residue was dissolved in 1,4-dioxane (100 mL) and water (100 mL), an aqueous solution of 4 mol/l potassium hydroxide (6.4 mL, 0.026 mol) was added thereto, and the mixture was stirred at 60° C. for 3.5 hours. DOWEX 50W was added to the reaction solution for neutralization and the solvent was then removed by distillation under reduced pressure. The residue was dissolved in dichloromethane (200 mL), N-acetylcysteamine (1.8 mL, 0.017 mol), hydrochloric acid/1-ethyl-3-(3′-dimethylaminopropyl)carbodiimide (3.2 g, 0.017 mol), and 4-dimethylaminopyridine (0.32 g, 0.0026 mol) were added thereto, and the mixture was stirred at 25° C. for 11 hours. After conventional post-processing, purification was carried out by chromatography on silica gel (eluted at hexane/ethyl acetate=1/1) to give Compound 3 (3.8 g, yield 74%; Table 1).

¹H NMR (CDCl₃) δ ppm; 5.80 (br s, 1H), 3.43 (dd, J=6.1, 12.5 Hz, 2H), 3.32 (dd, J=3.7, 5.3 Hz, 1H), 3.02 (t, J=6.6 Hz, 2H), 2.63 (dd, J=5.3, 9.9 Hz, 1H), 2.54 (dd, J=6.3, 9.4 Hz, 1H), 1.97 (s, 3H), 1.94 (m, 1H), 1.58 (m, 1H), 1.31-1.54 (m, 3H), 1.16 (m, 1H), 0.81-1.00 (m, 18H), 0.61 (q, J=7.6 Hz, 6H)

FABMS: m/z 404 (M+H)⁺

Molecular formula-based theoretical value: C₂₀H₄₁NO₃SiS=403

(4) Synthesis of Compound 4

Compound 3 (15 mg, 0.038 mmol) was dissolved in tetrahydrofuran (0.46 mL) and water (0.46 mL), acetic acid (0.45 mL) was added thereto, and the mixture was stirred at 0° C. for 2 hours. After conventional post-processing, purification was carried out by thin-layer chromatography (eluted at chloroform/methanol=10/1) to give Compound 4 (7.7 mg, yield 71%, purity 63%; Table 1).

¹H NMR (CDCl₃) δ ppm; 5.88 (br s, 1H), 3.64 (dd, J=6.0, 12.3 Hz, 2H), 3.20 (m, 1H), 3.02 (dt, J=1.8, 6.4 Hz, 2H), 2.58-2.72 (m, 2H), 2.06 (m, 1H), 1.97 (s, 3H), 1.43-1.70 (m, 3H), 1.33 (m, 1H), 1.28 (m, 1H), 0.82-0.95 (m, 9H)

FABMS: m/z 290 (M+H)⁺

Molecular formula-based theoretical value: C₁₄H₂₇NO₃S=289

### EXAMPLE 4

### Direct Production of 22,23-dihydroavermectin B1a

10 μl of spore suspension of Streptomyces avermitilis KS1mut obtained in Example 2 was inoculated in a test tube containing 10 ml of seed culture medium [a medium prepared by adjusting a solution containing 20 g of lactose, 15 g of Distillers solubles, 2.5 g of autolysed yeast (Difco), and 1,000 ml of distilled water at pH 7.2 with 2 mol/l potassium hydroxide, followed by high pressure steam sterilization at 121° C. for 15 minutes] and was cultured by shaking at 28° C. for 20 hours to obtain a seed culture. 0.4 ml of this seed culture was transferred to a conical flask (volume 100 ml) containing 20 ml of production medium [a medium prepared by subjecting 46 g of glucose, 24 g of peptonized milk (Oxoid), 2.5 g of autolysed yeast (Difco), 2.5 ml of polypropylene glycol #2000, and 1,000 ml of distilled water to high pressure steam sterilization at 121° C. for 15 minutes] and was cultured using a rotary shaker at 28° C. for 3 days at 220 rpm, then 50 μl of 1 mg/ml methanol solution of Compound 4 synthesized in Example 3 (containing 50% Compound 4) was added to the culture, and culturing by shaking was carried out again at 28° C. for 2 days. After the completion of culture, a double amount of methanol was added to the culture and the mixture was thoroughly stirred. Thereafter, the stirred product was centrifuged at room temperature at 3,000 rpm for 5 minutes to precipitate cells. The supernatant was then subjected to high-performance liquid chromatography (HPLC) analysis.

HPLC Analysis

Chromatography Condition

Chromatography condition

| | |
|---|---|
| Column: | Inertsil ODS-2 (4.6 × 150 mm, |
| | manufactured by GL Sciences Inc.) |
| Guard column: | Guard column E cartridge (4 × |
| | 10 mm, manufactured by GL |
| | Sciences Inc.) |
| Mobile phase: | acetonitrile:methanol:water = 70:10:20 |
| Flow rate: | 0.6 ml/min |
| Detection: | 246 nm |
| Temperature: | 55° C. |

The methanol extract of the culture was analyzed under the above conditions for analysis and, as a result, a peak was observed at a retention time of 21.7 minutes only in the culture extract to which Compound 4 was added. As a result of the analysis of 22,23-dihydroavermectin B1a under the equivalent condition, the retention time was the same, i.e., 21.7 minutes. When 22,23-dihydroavermectin B1a was determined as the standard, the yield of the substance exhibiting the retention time of 21.7 minutes, which was obtained from the culture extract, was 23.3 mg/L.

Three-dimensional HPLC analysis was carried out using a multi-wavelength detector MD-915 (manufactured by Jasco) and, as a result, the maximal absorption wavelength of the peak at the retention time of 21.7 minutes was 248 nm and the spectrum thereof coincided with that of 22,23-dihydroavermectin B11 a.

The peak at the retention time of 21.7 minutes was fractionated by HPLC and 5 mg of white powder was obtained and subjected to mass spectometry. The results were as follows.

m/z 873.5 (M+) C₄₈H₇₃O₁₄

This coincided with data of 22,23-dihydroavermectin B1a described in Ivermectin and Abamectin, William C. Campbell (1989).

As is apparent from the foregoing description, the substance, which was obtained by adding Compound 4 to Streptomyces avermitilis KS1mut and culturing the strain, was 22,23-dihydroavermectin B1a. In the above culturing with addition of compound 4, avermectin analog other than 22,23-dihydroavermectin B1a was not produced at all. Since the single production of 22,23-dihydroavermectin B1a was realized, the production of 22,23-dihydroavermectin B1a was shown to have been significantly facilitated.

### INDUSTRIAL APPLICABILITY

According to the present invention, 22,23-dihydroavermectin B1a, which is useful as a medicine, a veterinary drug, and a pesticide, can be directly produced. Therefore, the conventional processes for purifying avermectin B1a at an industrial level and for chemically modifying avermectin B1a, which are complicated and difficult, can be omitted. This can significantly decrease the cost and the time for the industrial production of 22,23-dihydroavermectin B1a. This also realizes the production of the formulation containing only 22,23-dihydroavermectin B1a, which is highly effective as medicines.

[Sequence Listing Free Text]

SEQ ID NO: 9 represents synthetic DNA based on the sequence between nucleotides 1954 and 1985 shown in SEQ ID NO: 1

SEQ ID NO: 10 represents synthetic DNA based on the sequence between nucleotides 1758 and 1776 shown in SEQ ID NO: 1

SEQ ID NO: 11 represents synthetic DNA based on the sequence between nucleotides 2710 and 2729 in SEQ ID NO: 1

What is claimed is:

1. A modified avermectin aglycon synthase comprising at least one domain with an eliminated or lowered activity, wherein the domain is selected from the group consisting of acyl carrier protein (ACP), β-ketoacyl ACP synthase (KS), acyltransferase (AT), β-ketoacyl ACP reductase (KR), dehydratase (DH), enoyl reductase (ER) and thioesterase (TE), which are involved in the synthesizing reaction of avermectin aglycon.

2. The modified avermectin aglycon synthase according to claim 1, wherein the modified avermectin aglycon synthase is derived from Streptomyces avermitilis.

3. The modified avermectin aglycon synthase according to claim 1, wherein the domain with an eliminated or lowered activity is selected from the group consisting of ATs, ACPs, KS1, AT1, KR1, ACP1, KS2, DH2 and KR2.

4. A modified avermectin aglycon synthase comprising an amino acid sequence wherein one or more amino acid residues are deleted, substituted or added in the amino acid sequence of the avermectin aglycon synthase consisting of the amino acid sequences shown in SEQ ID NOs: 4, 5, 6 and 7, and having an activity for producing 22,23-dihydroavermectin B1a or a derivative thereof when the modified avermectin aglycon synthase is contacted with an N-acetylcysteamine thioester compound.

5. The modified avermectin aglycon synthase according to claim 4, which contains a polypeptide consisting of the amino acid sequence shown in SEQ ID NO: 8.

6. The modified avermectin aglycon synthase according to claim 4, wherein the N-acetylcysteamine thioester compound is represented by formula (I): 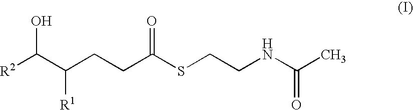
wherein R¹ and R², which may be the same or different, represent hydrogen, substituted or unsubstituted alkyl, substituted or unsubstituted alkenyl, substituted or unsubstituted aryl or substituted or unsubstituted heterocycle, or, R¹ and R², combined together, form substituted or unsubstituted cycloalkyl.

7. The modified avermectin aglycon synthase according to claim 6, wherein the N-acetylcysteamine thioester compound is represented by formula (I) in which R¹ is methyl and R² is sec-butyl.

8. A DNA which encodes the modified avermectin aglycon synthase according to any one of claims 1 to 7.

9. A DNA which comprises a DNA encoding a polypeptide consisting of the amino acid sequence shown in SEQ ID NO: 8.

10. A DNA which comprises a DNA consisting of the nucleotide sequence shown in SEQ ID NO: 3.

11. A DNA which hybridizes with the DNA according to any one of claims 8 to 10 under stringent conditions and encodes a polypeptide having an activity for producing 22,23-dihydroavermectin B1a or a derivative thereof when the modified avermectin aglycom synthase is contacted with the N-acetylcysteamine thioester compound.

12. A recombinant DNA which is obtained by ligating the DNA according to any one of claims 8 to 11 with a vector.

13. A transformant which is obtained by introducing the recombinant DNA according to claim 12 into a host cell.

14. The transformant according to claim 13, wherein the host cell is a microorganism.

15. The transformant according to claim 14, wherein the microorganism belongs to the genus Streptomyces.

16. The transformant according to claim 15, wherein the microorganism belonging to the genus Streptomyces is Streptomyces avermitilis.

17. The transformant according to claim 16, which is Streptomyces avermitilis KS1mut.

18. An N-acetylcysteamine thioester compound, which is a substrate compound for the modified avermectin aglycon synthase according to any one of claims 1 to 7 and converted to 22,23-dihydroavermectin B1a or a derivative thereof when the compound is contacted with the modified avermectin aglycon synthase.

19. An N-acetylcysteamine thioester compound represented by formula (I): 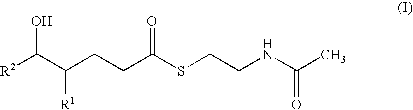
wherein R¹ and R² which may be the same or different, represent hydrogen, substituted or unsubstituted alkyl, substituted or unsubstituted alkenyl, substituted or unsubstituted aryl or substituted or unsubstituted heterocycle, or, R¹ and R², combined together, form substituted or unsubstituted cycloalkyl.

20. The N-acetylcysteamine thioester compound according to claim 19, which is represented by formula (I), wherein R¹ is methyl and R² is sec-butyl.

21. A process for producing an N-acetylcysteamine thioester compound which is characterized by employing a compound represented by formula (II): 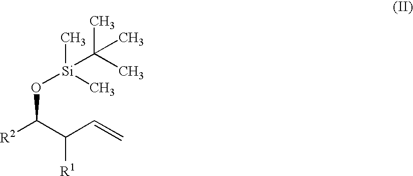
wherein R¹ and R², which may be the same or different, represent hydrogen, substituted or unsubstituted alkyl, substituted or unsubstituted alkenyl, substituted or unsubstituted aryl or substituted or unsubstituted heterocycle, or, R¹ and R², combined together, form substituted or unsubstituted cycloalkyl as a starting material, and including a reaction step of adding N-acetylcysteamine.

22. The process for producing an N-acetylcysteamine thioester compound according to claim 21, which is characterized by employing, as a starting material, a compound represented by formula (II): 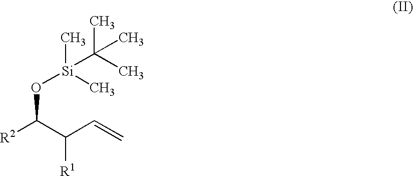
wherein R¹ and R², which may be the same or different, represent hydrogen, substituted or unsubstituted alkyl, substituted or unsubstituted alkenyl, substituted or unsubstituted aryl or substituted or unsubstituted heterocycle, or, R¹ and R², combined together, form substituted or unsubstituted cycloalkyl, and comprising the steps of:
(a) ozone-oxidating the compound, and thereafter adding carbon chains by the Wittig reaction;
(b) deprotecting t-butyldimethylsilyl group of the compound obtained in step (a) and reintroducing another protecting group using chlorotriethylsilane;
(c) reducing α-β unsaturated carbon bond of the resultant compound in the presence of a palladium-carbon catalyst, hydrolyzing an ester with potassium hydroxide, neutralizing the reaction mixture, and adding N-acetylcysteamine in the presence of a condensing agent to obtain a thioester compound; and
(d) removing the protecting group by adding acetic acid to the thioester compound.

23. A process for producing a modified avermectin aglycon synthase, comprising the steps of:
culturing the transformant according to any one of claims 13 to 17 in a medium until a modified polypeptide having an activity of a avermectin aglycon synthase is produced and accumulated in the culture; and
collecting the polypeptide from the culture.

24. A process for producing 22,23-dihydroavermectin B1a or a derivative thereof, comprising the steps of:
contacting a culture of the transformant according to any one of claims 13 to 17 or a treated product thereof or the synthase according to any one of claims 1 to 7 with the N-acetylcysteamine thioester compound according to claim 18 in a medium; and
collecting 22,23-dihydroavermectin B1a or a derivative thereof produced and accumulated in the medium.

25. A process for producing 22,23-dihydroavermectin B1a or a derivative thereof, characterized in that an N-acetylcysteamine thioester compound is employed as a substrate compound for the modified avermectin aglycon synthase according to any one of claims 1 to 7.
